(19) United States
(12) Patent Application Publication
(10) Pub. No.: US 2014/0018779 A1
(43) Pub. Date: Jan. 16, 2014

(54) TELEMEDICINE CARE SYSTEM

(71) Applicants: Kai R. Worrell, Golden Valley, MN (US); Benjamin R. Van Sistine, Minneapolis, MN (US); Daniel Darst, Zimmerman, MN (US); Maria Elizabeth LuLu Petrina, Minneapolis, MN (US)

(72) Inventors: Kai R. Worrell, Golden Valley, MN (US); Benjamin R. Van Sistine, Minneapolis, MN (US); Daniel Darst, Zimmerman, MN (US); Maria Elizabeth LuLu Petrina, Minneapolis, MN (US)

(73) Assignee: Worrell, Inc., Minneapolis, MN (US)

(21) Appl. No.: 13/624,962

(22) Filed: Sep. 23, 2012

Related U.S. Application Data
(60) Provisional application No. 61/670,749, filed on Jul. 12, 2012.

(51) Int. Cl. A61B 19/00

(52) U.S. Cl. CPC A61B 19/00
USPC 606/1

(57) ABSTRACT

A telemedicine care system connects patients with remote healthcare professionals using a patient care unit at the location of the patient. The patient care unit provides video, audio, and data communication capabilities by integrating a computing device, various communication devices, and medical instruments into a single unit. Additional systems and methods are also disclosed.

### CROSS-REFERENCE TO RELATED APPLICATION(S)

This application claims priority to U.S. Patent Application Ser. No. 61/670,749, filed on Jul. 12, 2012, and titled TELEMEDICINE CARE SYSTEM, the disclosure of which is hereby incorporated by reference in its entirety.

### BACKGROUND

The typical healthcare scenario involves an in-person interaction between a patient and a healthcare professional. During the interaction, the patient may describe symptoms of concerns, and the healthcare professional may evaluate the patient to consider an appropriate course of action.

Oftentimes a patient schedules an appointment with a healthcare professional when such an appointment may not have been medically necessary. Unusual or unexpected symptoms can prompt a fear response in a patient, who instinctively responds by scheduling an appointment. In other cases, symptoms may indeed indicate a serious and urgent condition. Unfortunately, healthcare providers can be overwhelmed by such requests, and the patient experiencing such symptoms may not be able to obtain an appointment for some time into the future.

Some insurance providers have responded by providing telephone advice services to patients. When a patient experiences concerning symptoms, the patient (or another care provider such as a parent, spouse, relative or friend) telephones the telephone advice service that is staffed by professional nursing staff. The nurse listens to a description of the symptoms and asks questions intended to determine the severity and urgency of the condition. If the nurse can determine that the symptoms are unlikely to be severe and urgent, the nurse provides care suggestions and advice on additional symptoms to watch for. The nurse may also suggest that the patient schedule an appointment with a healthcare professional.

Telephone advice services are very limited in the services that the nursing staff can provide. One of the limitations of such services is that the information available to the nurse is limited to the descriptions provided by the patient or other care provider. It is therefore prone to error, due to potential misperceptions of the patient or care provider, failure to properly communicate an accurate description of the symptoms, failure to identify important symptoms, and the like. Also, the nurse typically has no access to any historical medical records for the patient, and is again limited to the medical history provided by and known by the patient or care provider. As a result of these and other limitations, the services provided by telephone advice services are limited, and often result in a recommendation that an appointment be scheduled with a healthcare professional.

### SUMMARY

In general terms, this disclosure is directed to a telemedicine care system. In one possible configuration and by non-limiting example, the telemedicine care system includes a patient care unit that provides audio and/or video conferencing capabilities and diagnostic instruments for remote examination of and interaction with a patient by a healthcare professional.

One aspect is a mobile patient care unit. The unit includes a housing comprising a base portion; a display portion; and a hinge pivotally connecting the display portion to the base portion to permit the display portion to pivot with respect to the base portion between a closed position and an open position, wherein the housing encloses an interior space when the housing is in the closed position. The unit also includes a touch sensitive display positioned in the display portion of the housing; a video camera positioned in the display portion of the housing; electronics at least partially contained by the housing and at least one medical instrument. The electronics include: at least one processing device; a microphone; a speaker; a wireless communication device configured to wirelessly communicate across a digital data communication network; a cellular communication device configured to wirelessly communicate using a cellular communication network; and an instrument interface. The at least one medical instrument is configured to be stored within the interior space of the base portion of the housing, wherein the medical instrument is at least partially controlled by the electronics through the instrument interface.

Another aspect is a patient care unit that permits a patient to interact with a remote health care professional, the patient care unit comprising: a housing defining at least one electronics enclosure and an interior space; electronic components at least partially contained within the at least one electronics enclosure, and comprising at least one main processing device and at least one separate instrument processing device in data communication with the at least one main processing device; at least one video camera in data communication with the electronic components and configured to capture a video of the patient for transmission to the remote healthcare professional; and at least one instrument configured to evaluate an aspect of the patient's current medical condition and generate an output, wherein the output is processed by the instrument processing device separate from the main processing device.

Another aspect is a care system server configured to communicate data between a patient care unit at the location of a patient and a computing device of a healthcare professional, wherein the computing device and the healthcare professional are located remote from the patient, the care system server comprising: at least one processing device; and at least one computer readable storage device, wherein the at least one computer readable storage device stores data instructions, which when executed by the at least one processing device, cause the processing device to generate: a patient care unit interface configured to receive data from the patient care unit; an electronic medical records interface configured to retrieve data from an electronic medical record associated with the patient; and a healthcare professional interface configured to transmit at least some of the data from the patient care unit and at least some of the data from the electronic medical record of the patient to the computing device of the healthcare professional for presentation to the healthcare professional.

Additional aspects are described herein.

### BRIEF DESCRIPTION OF THE DRAWINGS

FIG. 1 is a schematic block diagram of an exemplary telemedicine system according to the present disclosure.

FIG. 2 is a perspective diagram illustrating an exemplary environment in which a patient care unit of the telemedicine system shown in FIG. 1 is used.

FIG. 3 is an isometric view illustrating an example of the patient care unit shown in FIG. 2 arranged in a closed configuration.

FIG. 4 is another isometric view of an example of the patient care unit shown in FIG. 2 when arranged in an open configuration.

FIG. 5 is another isometric view of an example of the patient care unit shown in FIG. 2.

FIG. 6 illustrates an exemplary collection of instruments of the patient care unit shown in FIG. 2.

FIG. 7 is a schematic block diagram illustrating an example of the electronic components of the patient care unit shown in FIG. 2.

FIG. 8 is a schematic block diagram of an exemplary configuration of a care system server of the telemedicine system shown in FIG. 1.

FIG. 9 illustrates an exemplary station of a call center of the telemedicine system shown in FIG. 1.

FIG. 10 is another view of the exemplary station of the call center shown in FIG. 9.

FIG. 11 illustrates an exemplary architecture of a healthcare professional's computing device of the telemedicine system shown in FIG. 1.

FIG. 12 is a screen shot illustrating an example of the healthcare professional user interface, such as displayed on the computing device shown in FIG. 11.

FIG. 13 is a screen shot of an example user interface of the patient care unit.

FIG. 14 is a screen shot of an example user interface of the patient care unit.

FIG. 15 is a screen shot of an example user interface of the patient care unit.

FIG. 16 is a screen shot of an example user interface of the patient care unit.

FIG. 17 is a screen shot of an example user interface of the patient care unit.

FIG. 18 is a screen shot of an example user interface of the patient care unit.

FIG. 19 is a screen shot of an example user interface of the patient care unit.

FIG. 20 is a screen shot of an example user interface of the patient care unit.

FIG. 21 is a screen shot of an example user interface of the patient care unit.

FIG. 22 is a screen shot of an example user interface of the patient care unit.

FIG. 23 is a screen shot of an example user interface of the patient care unit.

FIG. 24 is a screen shot of an example user interface of the patient care unit.

FIG. 25 is a screen shot of an example user interface of the patient care unit.

FIG. 26 is a screen shot of an example user interface of the patient care unit.

FIG. 27 is a screen shot of an example user interface of the patient care unit.

FIG. 28 is a screen shot of an example user interface of the patient care unit.

FIG. 29 is a screen shot of an example user interface of the patient care unit.

FIG. 30 is a screen shot of an example user interface of the patient care unit.

FIG. 31 is a screen shot of an example user interface of the patient care unit.

FIG. 32 is a screen shot of an example user interface of the patient care unit.

FIG. 33 is a screen shot of an example user interface of the patient care unit.

FIG. 34 is a perspective view of another example of the patient care unit.

FIG. 35 is a perspective view of another example patient care unit.

FIG. 36 is another perspective view of the patient care unit shown in FIG. 35.

FIG. 37 is illustrates an example of a pill organizer of the patient care unit shown in FIG. 35.

FIG. 38 is a schematic cross-sectional view of a receptacle of the pill organizer shown in FIG. 37.

FIG. 39 is another schematic cross-sectional view of the receptacle shown in FIG. 38.

FIG. 40 is a schematic diagram illustrating exemplary communication devices of a patient care unit.

FIG. 41 is a screen shot of an example admin page of an example user interface displayed on the patient care unit.

FIG. 42 is a screen short of an example Wi-Fi settings page of the user interface shown in FIG. 41.

FIG. 43 is a screen shot of an example home page of a user interface displayed on the patient care unit.

FIG. 44 is a screen shot of an example validation screen of the user interface shown in FIG. 43.

FIG. 45 is a screen shot of the live conference page of the user interface shown in FIG. 43.

FIG. 46 is a screen shot of an example patient information page for an asthma action plan user interface as displayed on the patient care unit.

FIG. 47 is a screen shot of a first condition page of the user interface shown in FIG. 46.

FIG. 48 is a screen shot of a medication page of the user interface shown in FIG. 46.

FIG. 49 is a screen shot of an additional actions page of the user interface shown in FIG. 46.

FIG. 50 is a screen shot of a follow up page of the user interface shown in FIG. 46.

FIG. 51 is a screen shot of an example condition prompting page of the user interface as displayed on a patient care unit.

FIG. 52 is a screen shot of an example treatment page of the user interface shown in FIG. 51.

FIG. 53 is a screen shot of an example control test selection page of the user interface as displayed by the patient care unit.

FIG. 54 is a screen shot of a test question page of the user interface shown in FIG. 53.

FIG. 55 is a screen shot of another test question page of the user interface shown in FIG. 53.

FIG. 56 is a screen shot of another test question page of the user interface shown in FIG. 53.

FIG. 57 is a screen shot illustrating the display of an asthma control test score.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

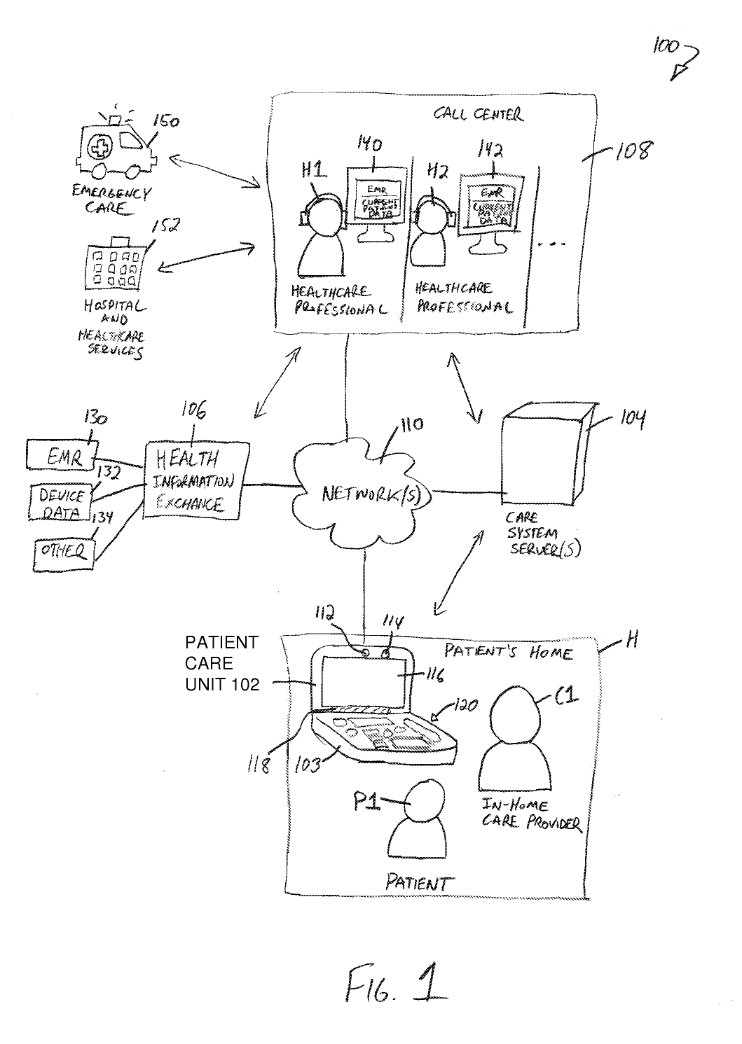

FIG. 1 is a schematic block diagram of an exemplary telemedicine care system 100. In this example, the telemedicine care system 100 includes a patient care unit 102, care system server(s) 104, a health information exchange 106, and a call center 108. One or more data communication networks 110 are utilized for communication of data between the various components of the telemedicine care system 100.

The patient care unit 102 is provided for use at the location of the patient P remote from the healthcare professionals (H1 and H2). In this example, the patient is at home H, and may be accompanied by an in-home care provider C1, such as a family member, friend, or other care provider. The telemedicine care system 100 can include many patient care units 102 at many different locations for use by many different patients.

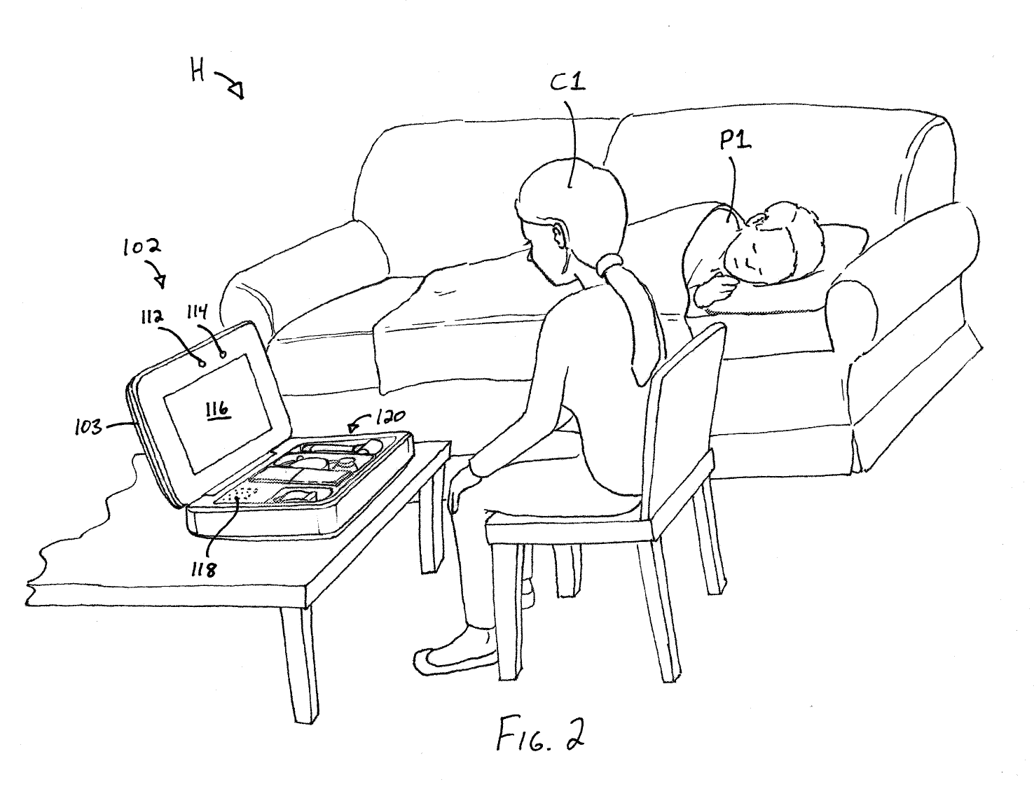

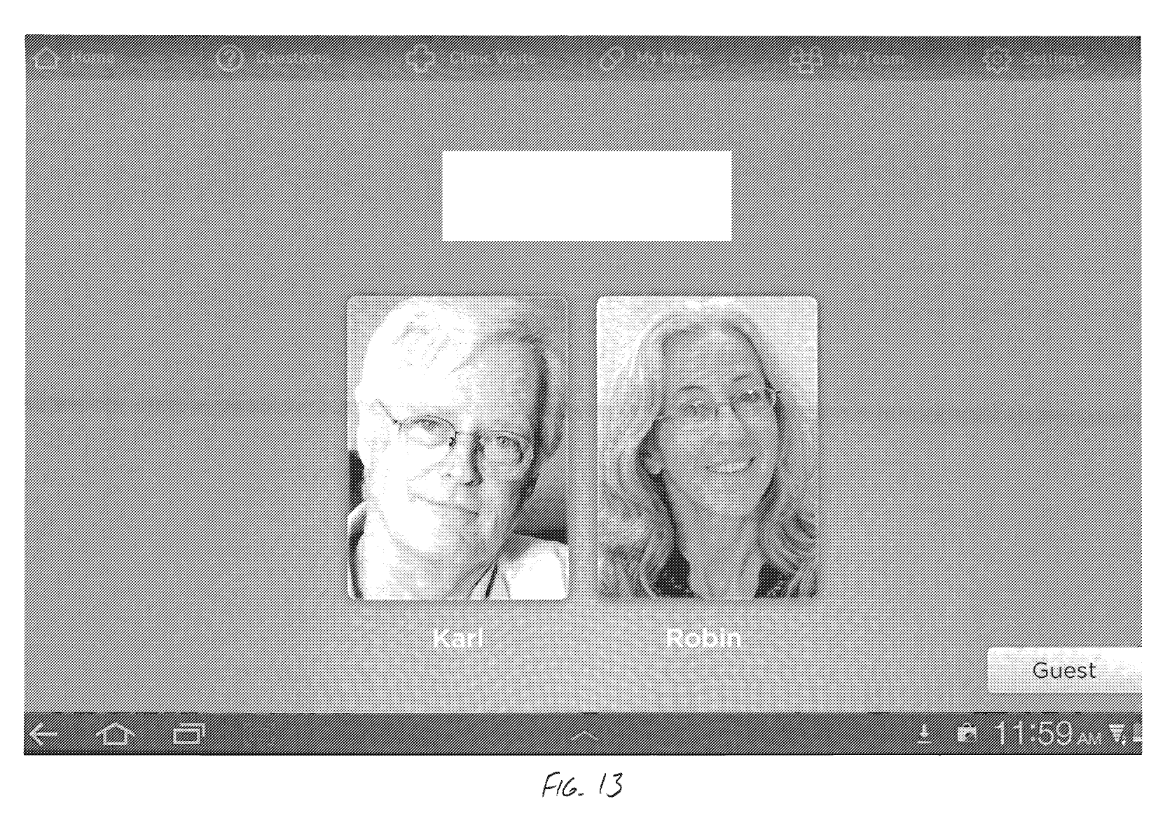

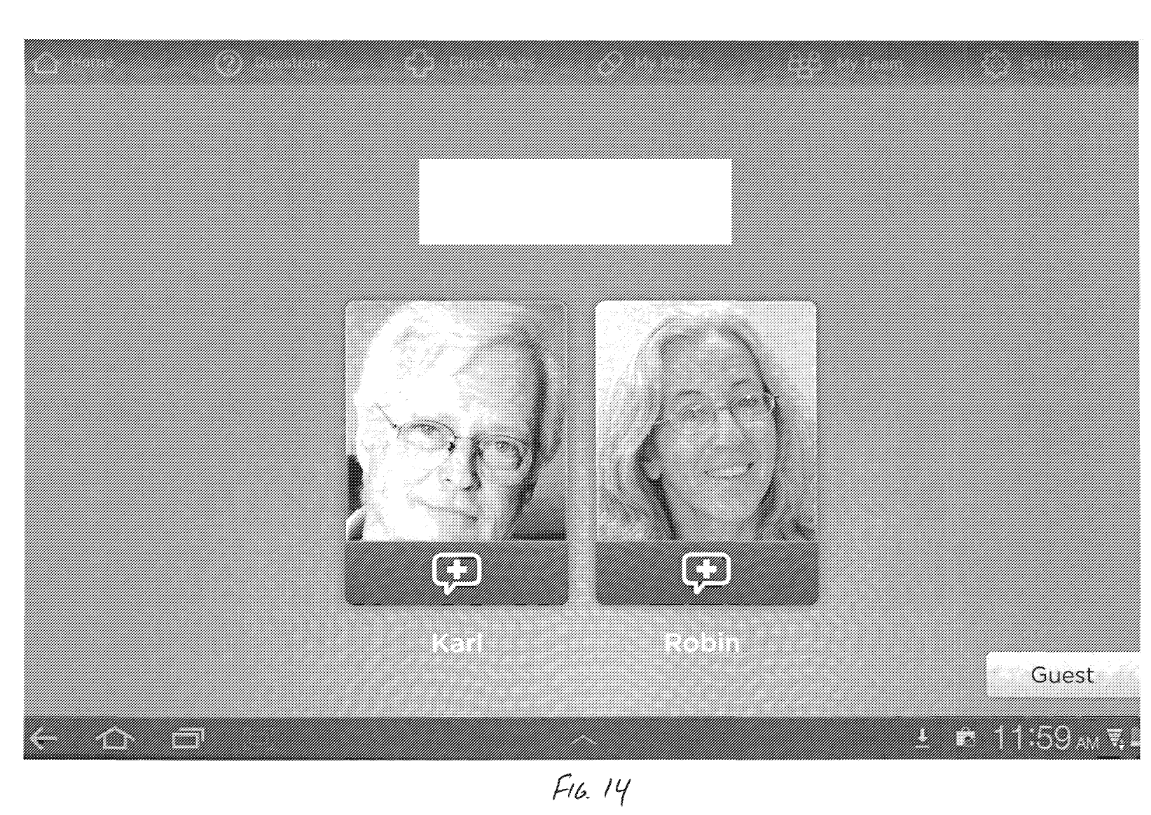

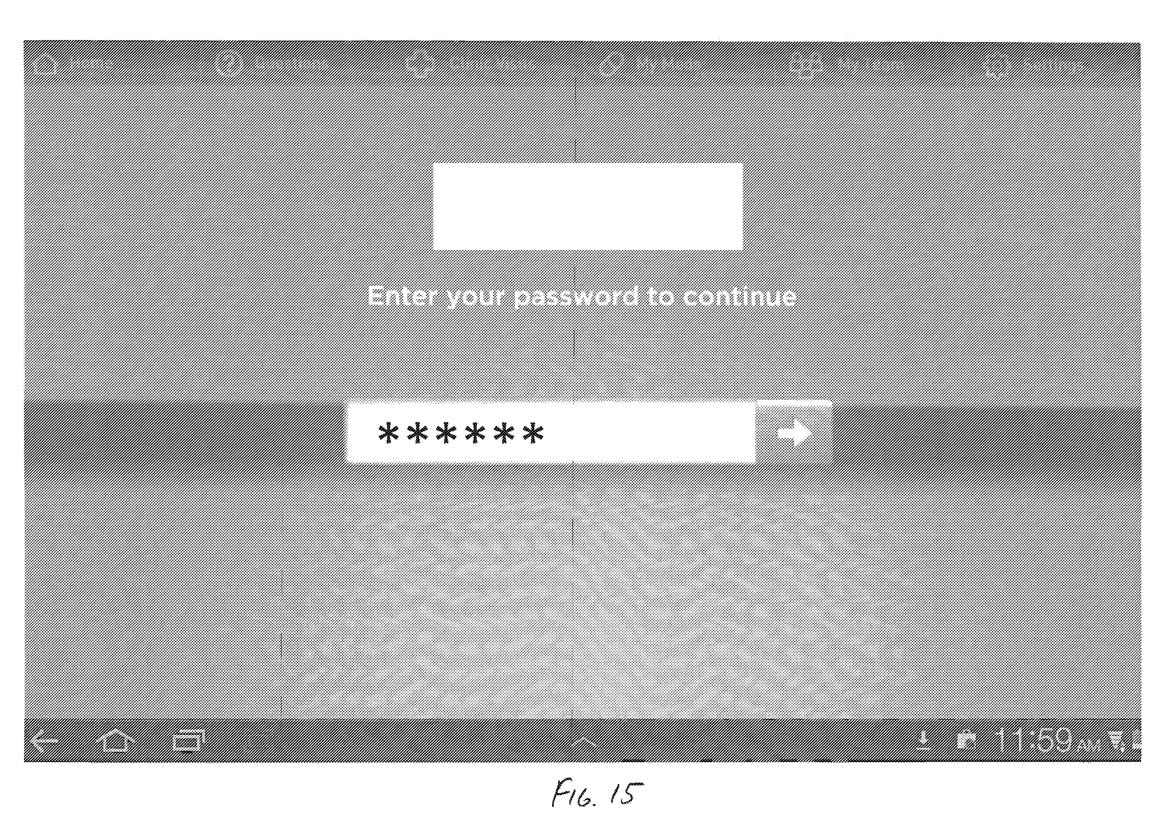

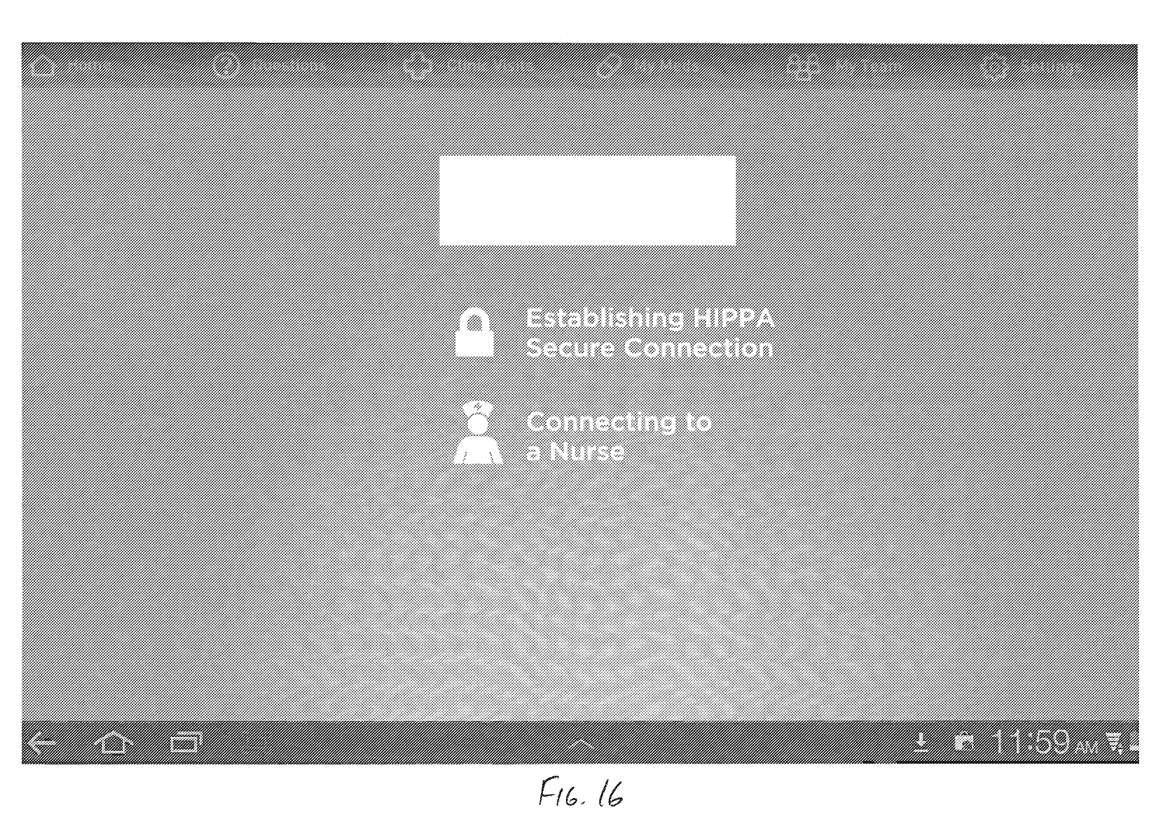

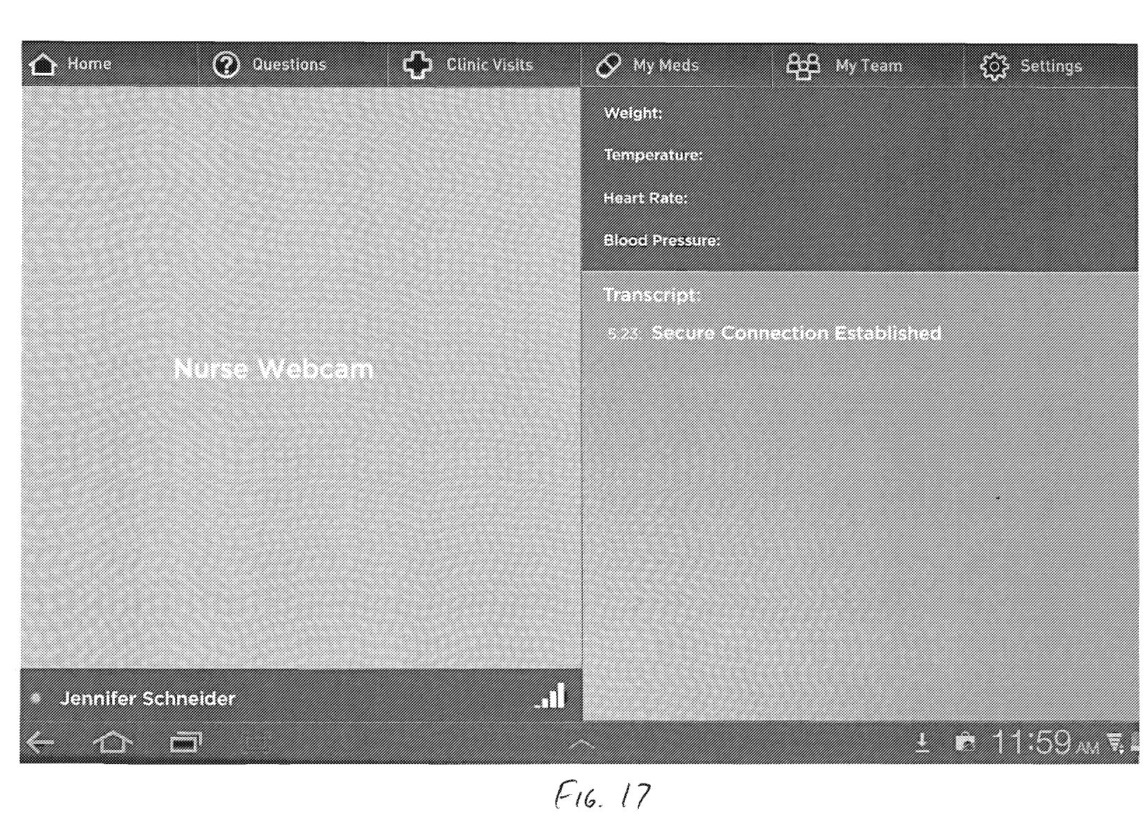

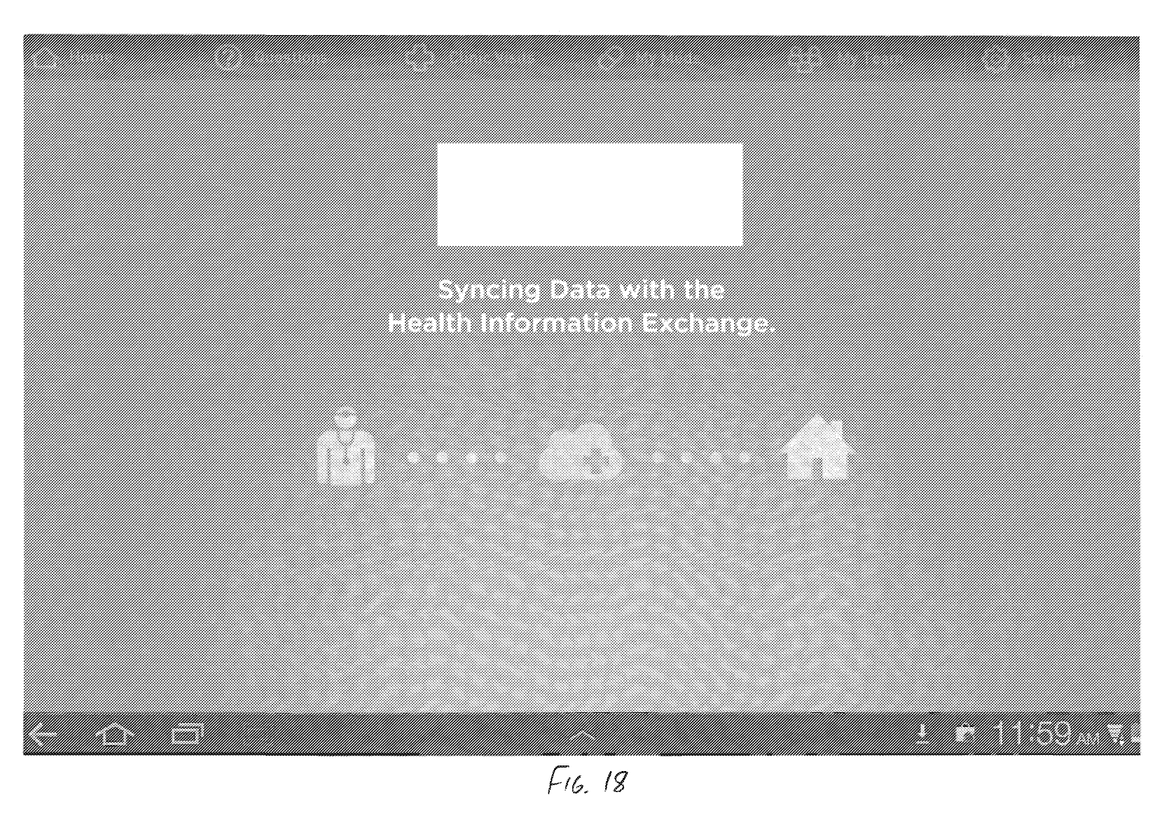

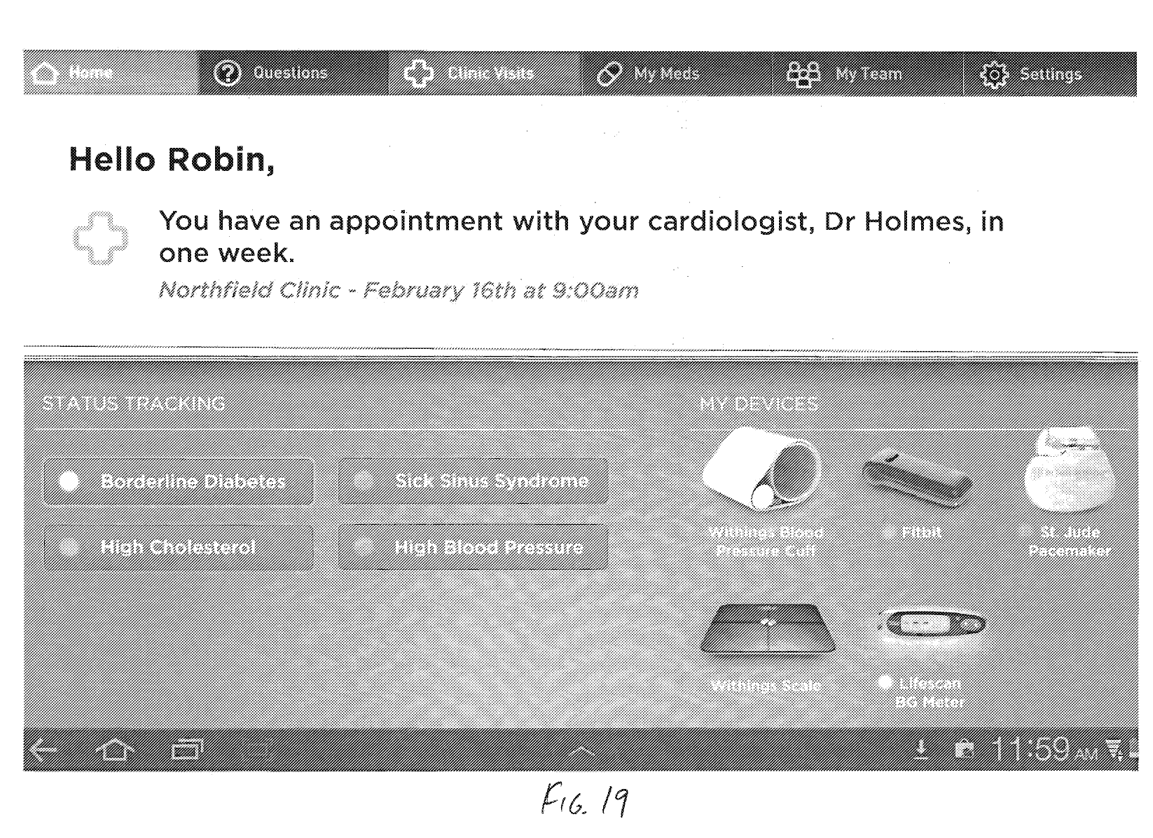

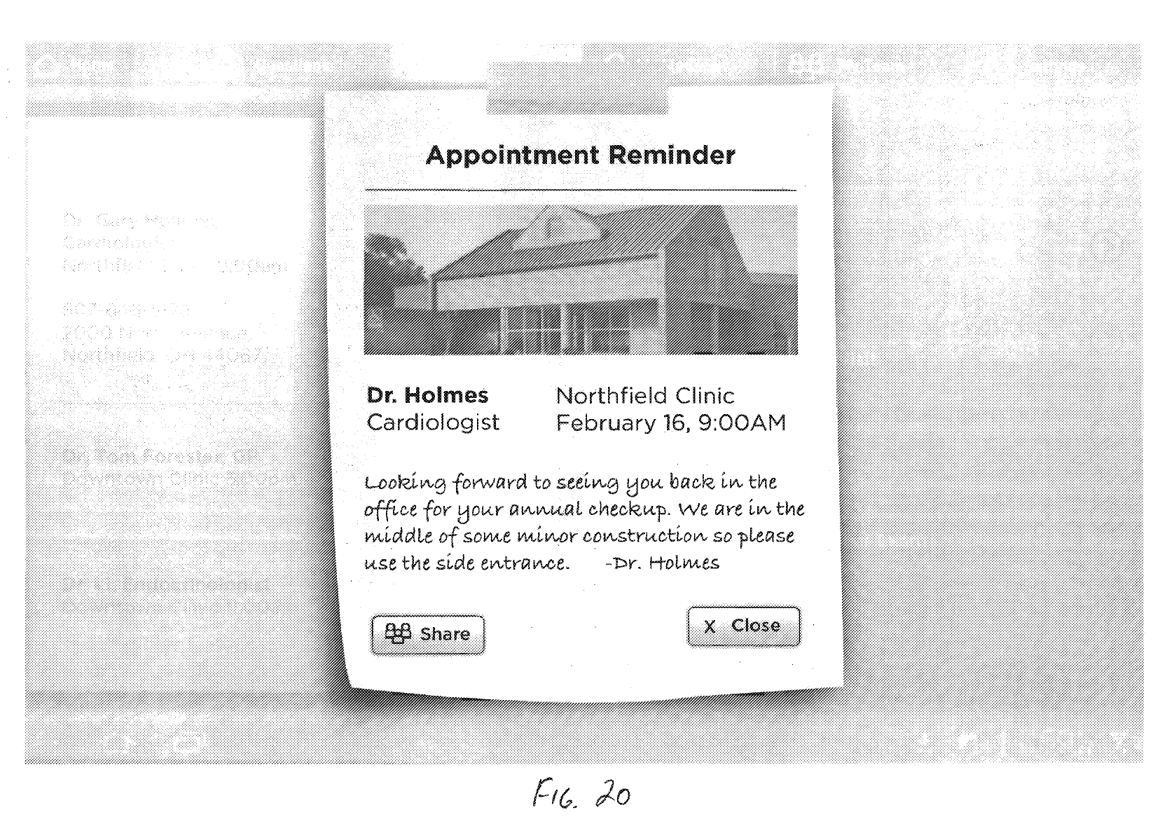

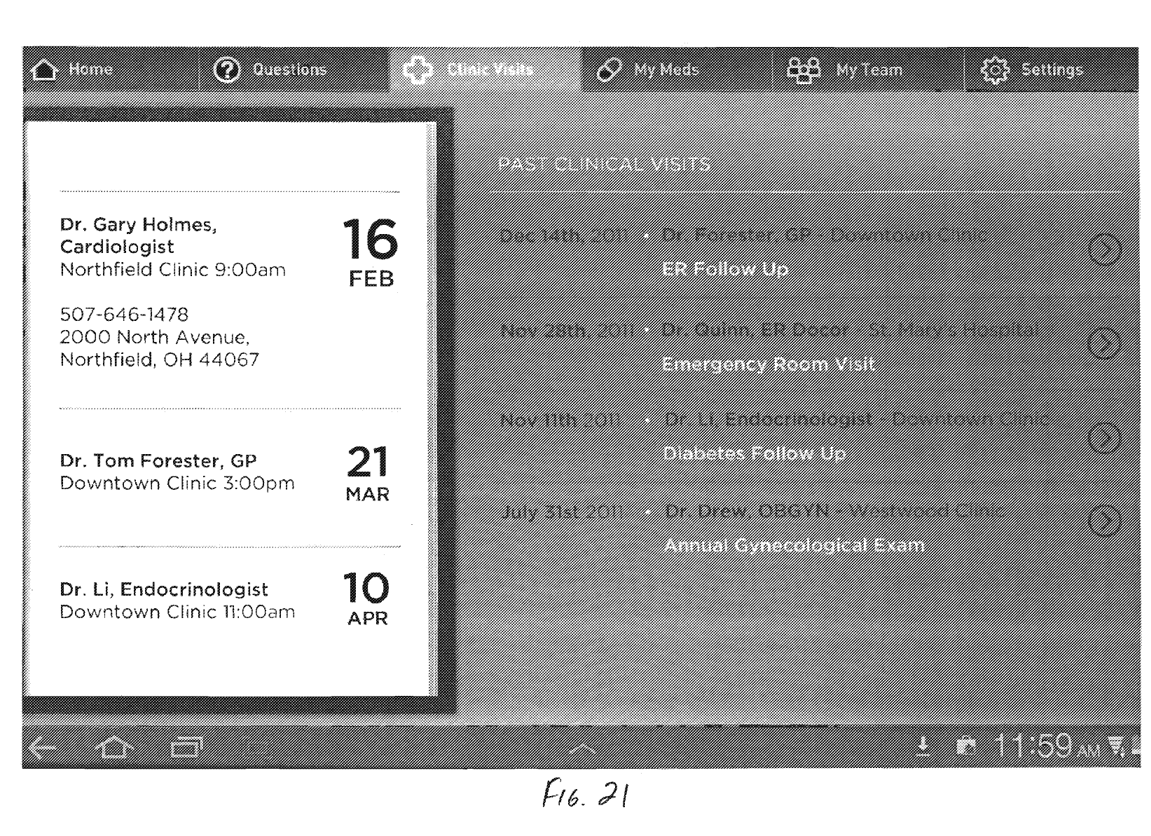

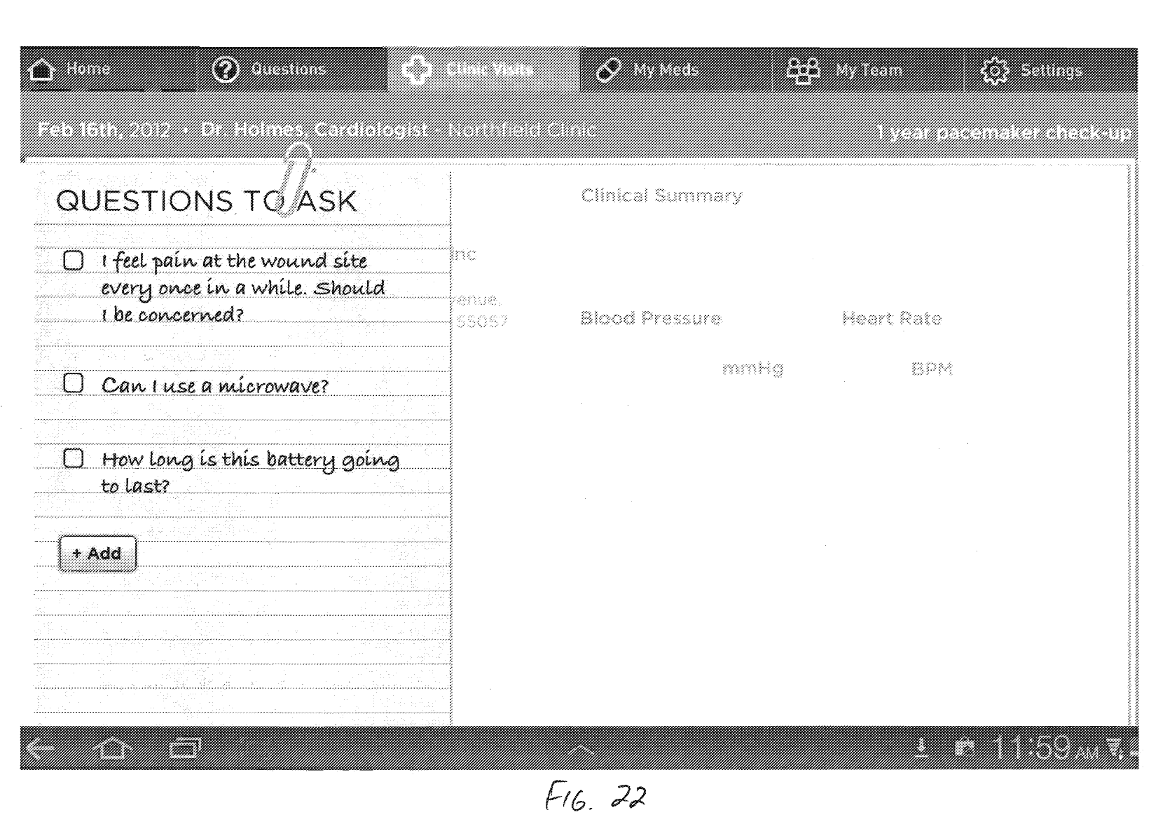

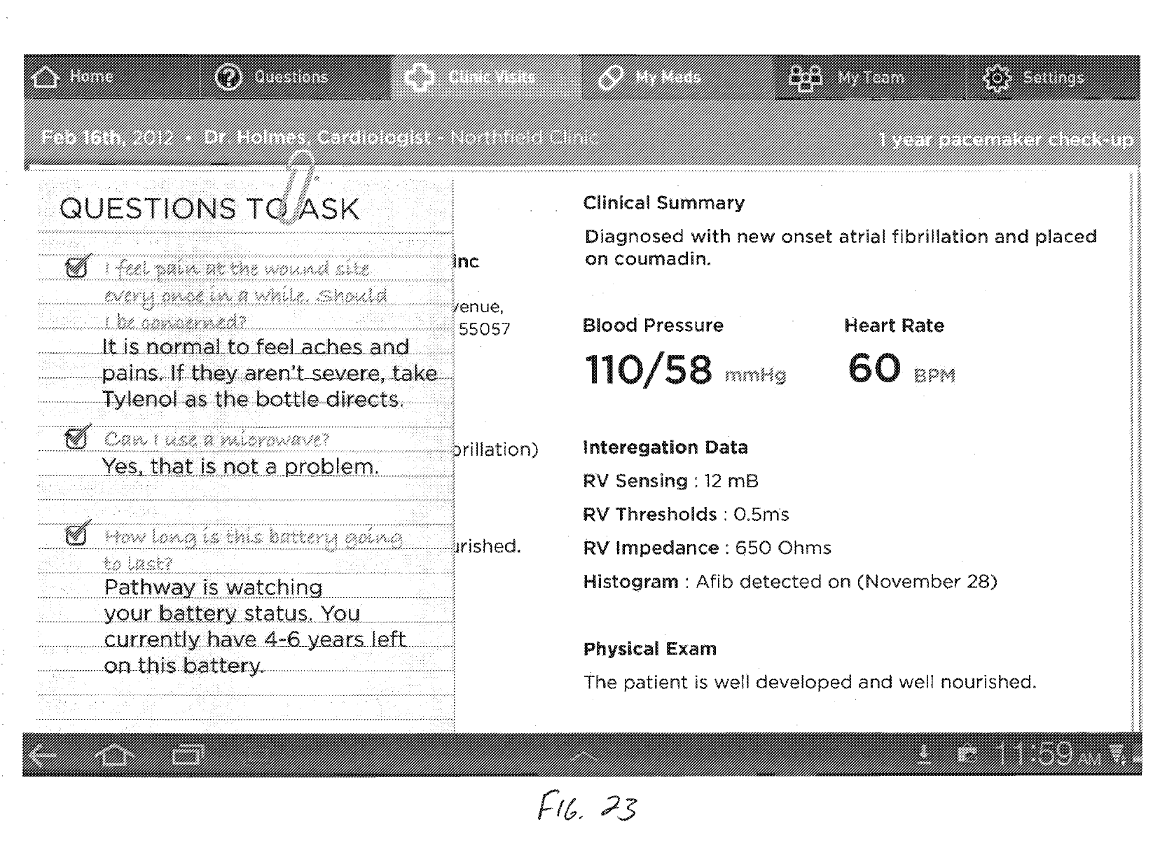

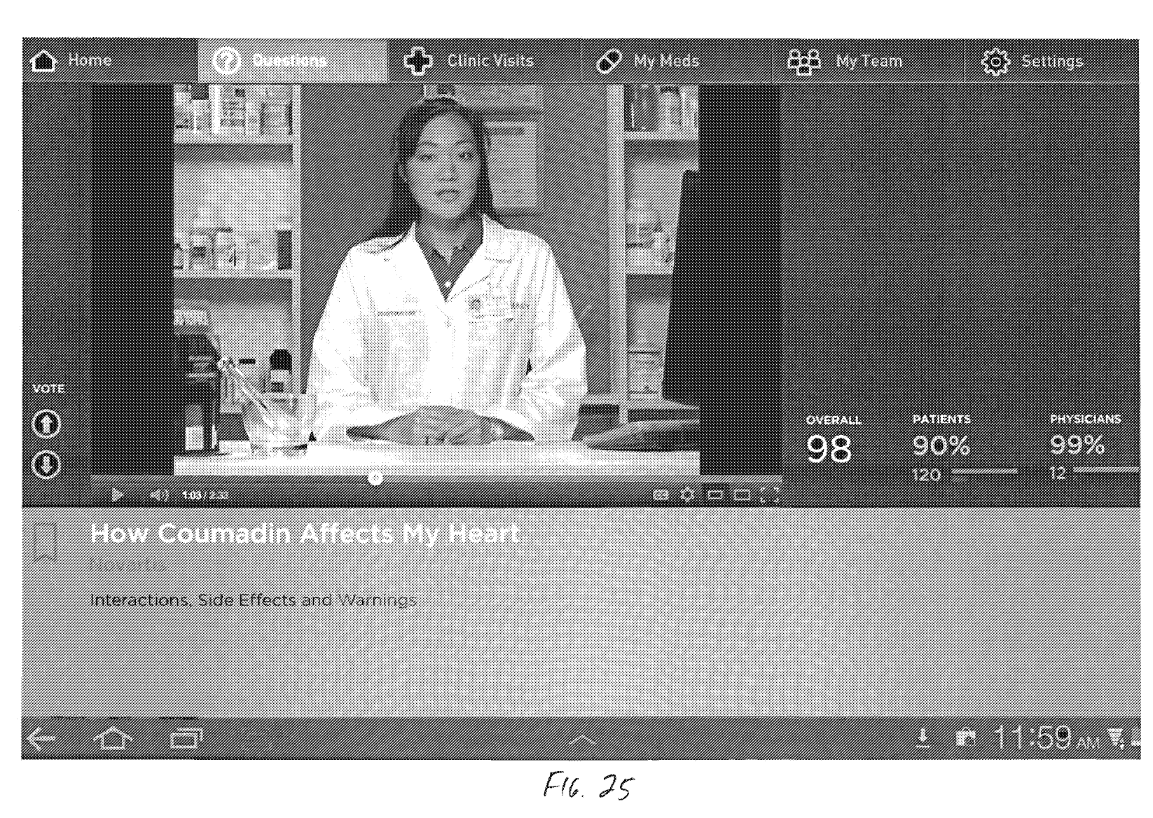

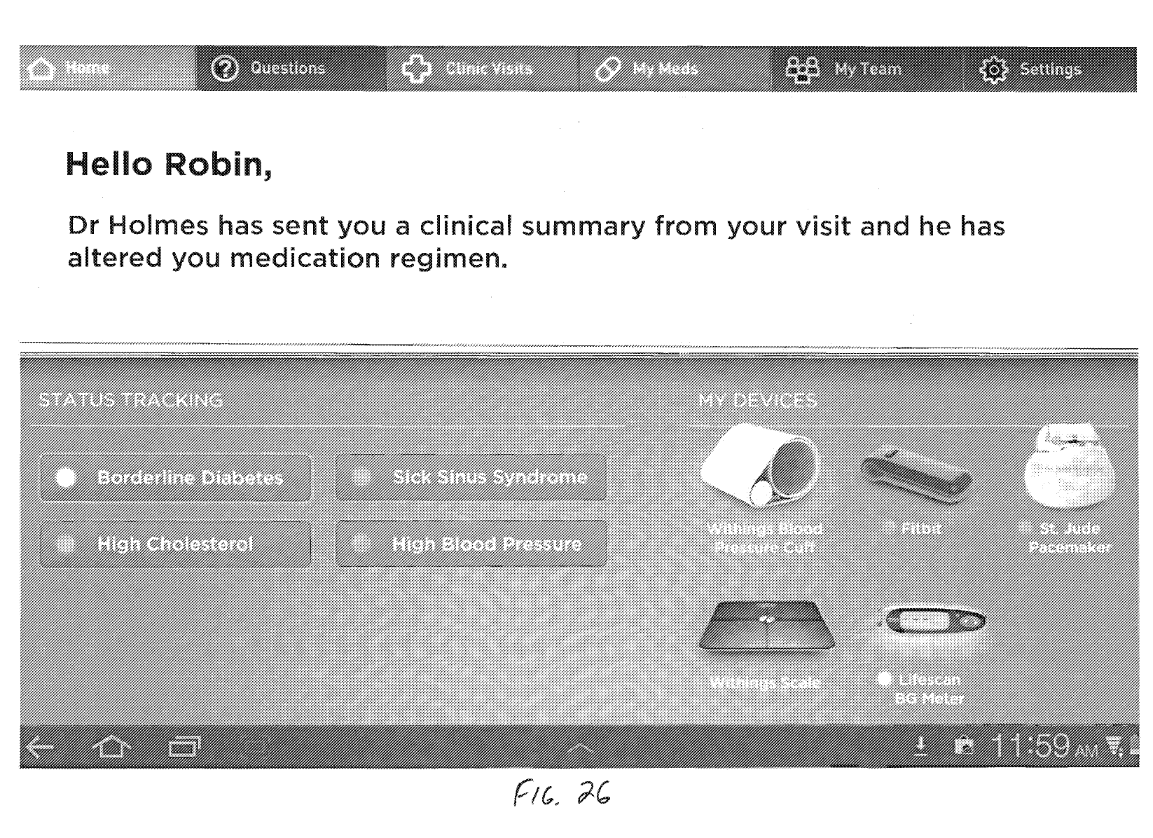

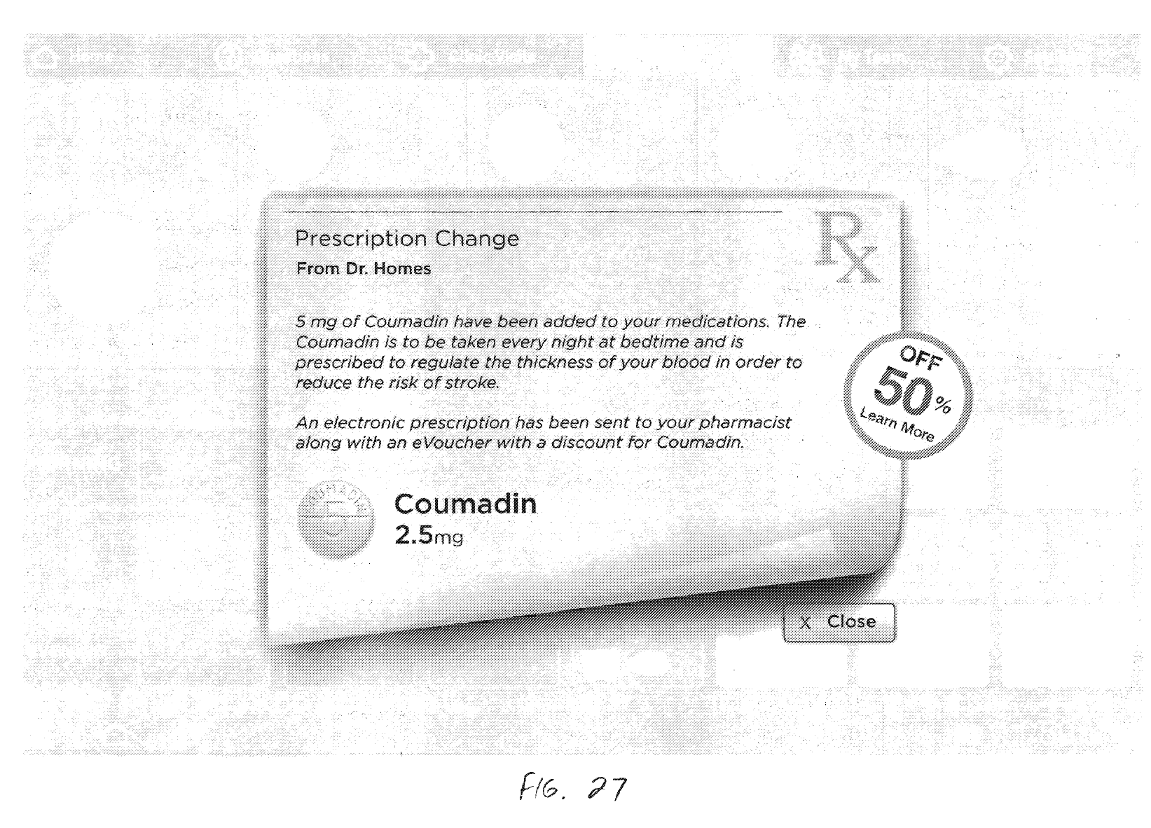

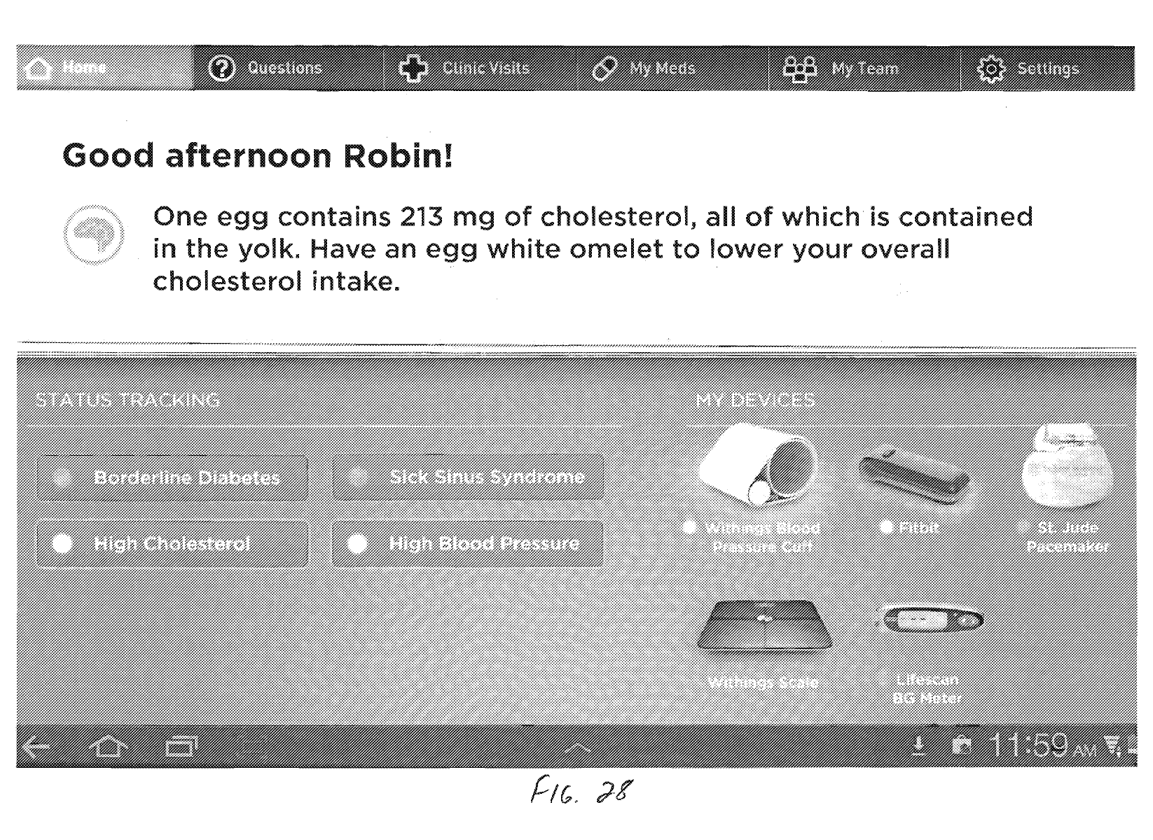

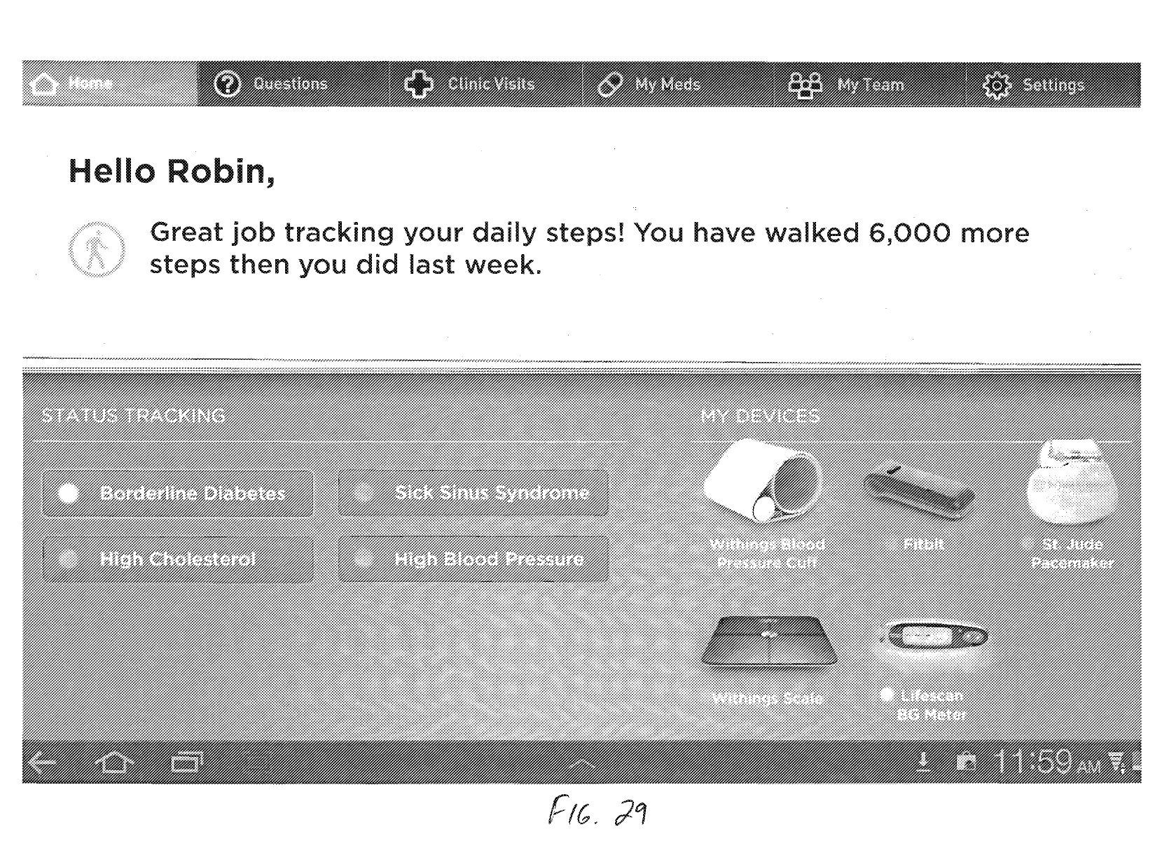

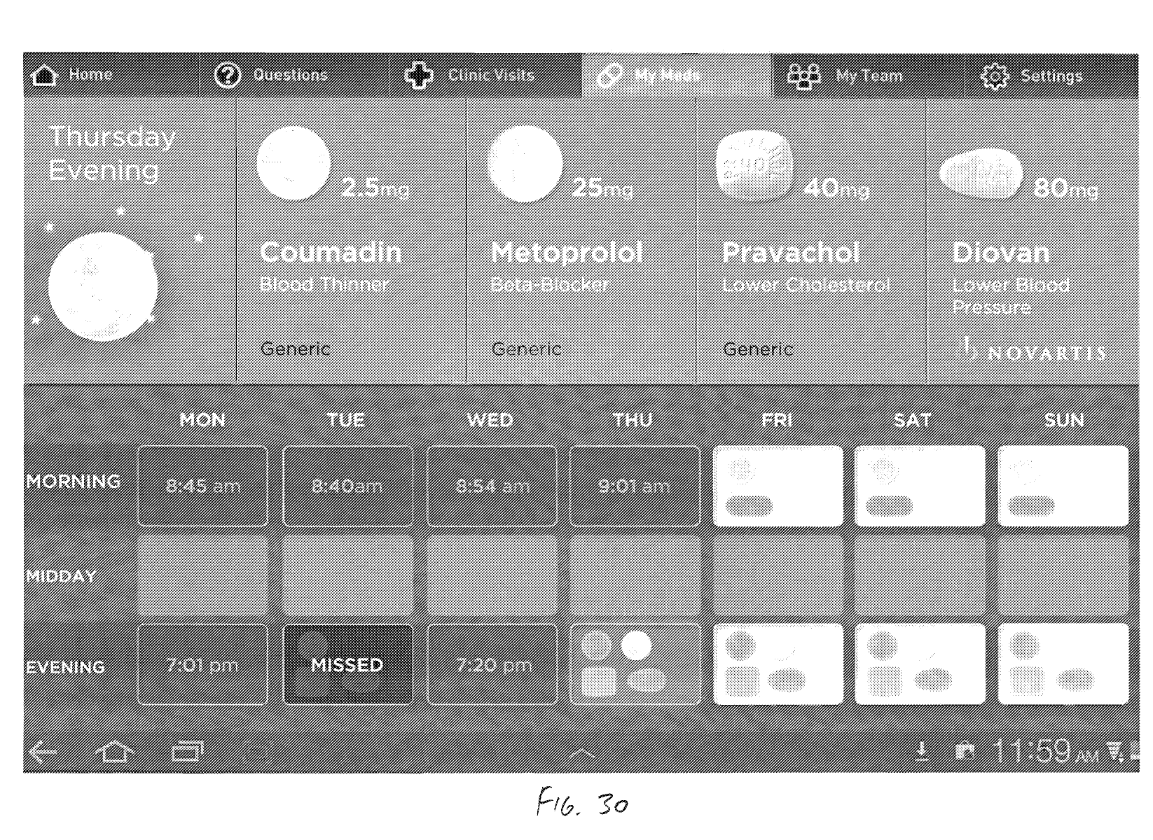

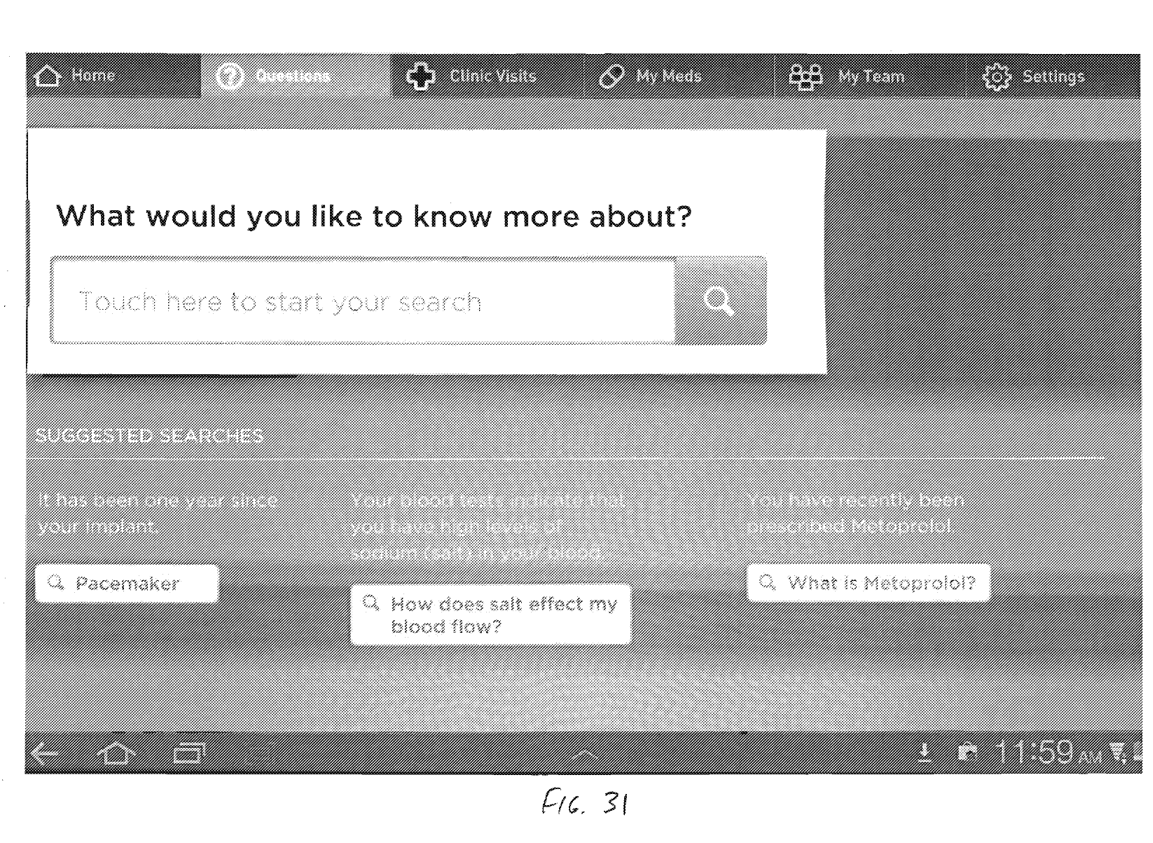

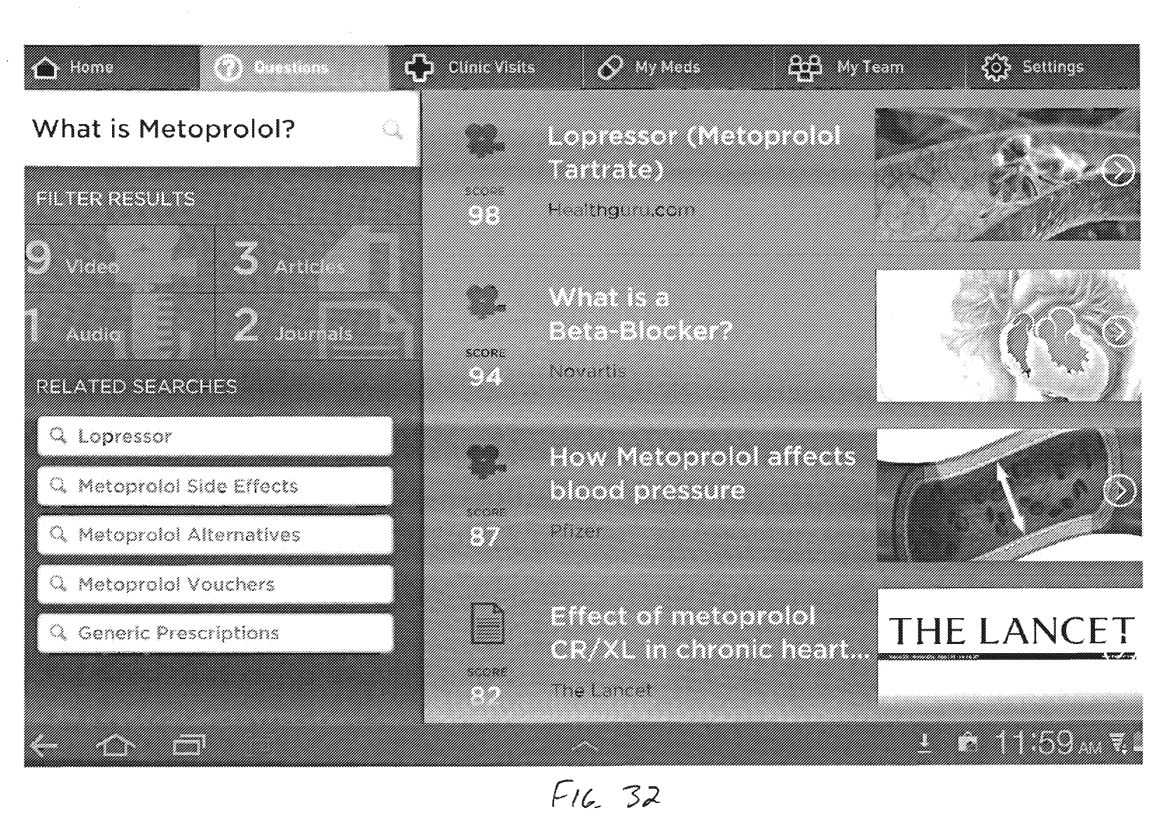

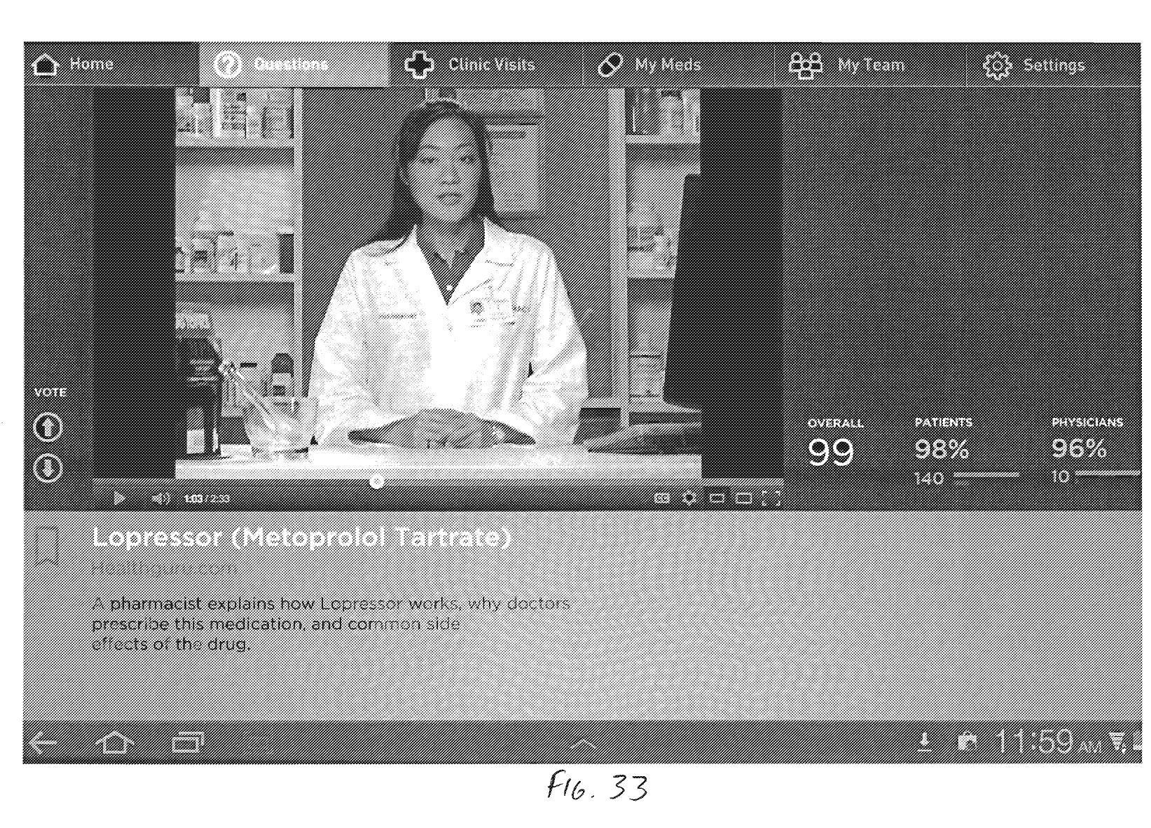

In some embodiments, the patient care unit 102 includes a patient care device 103 and one or more diagnostic instruments 120. The patient care device 103 includes, for example, at least one video camera 112, a microphone 114, a display 116, a speaker 118, and other electronics. The video camera 112 and microphone 114 permit communication from the patient P or care provider C1 to the healthcare professionals H1, H2 in the call center 108. The display 116 and speaker 118 permit communication from the healthcare professionals H1, H2 in the call center 108 to the patient P or care provider C1. Diagnostic instruments 120 are provided in some embodiments to collect data regarding the patient's current condition. An example of a diagnostic instrument is a blood pressure cuff. Other examples of diagnostic instruments are described herein. The patient care unit 102 is illustrated and described in more detail with reference to FIGS. 2-7 herein. In addition, an exemplary user interface of the patient care unit 102 is illustrated in FIGS. 13-33.

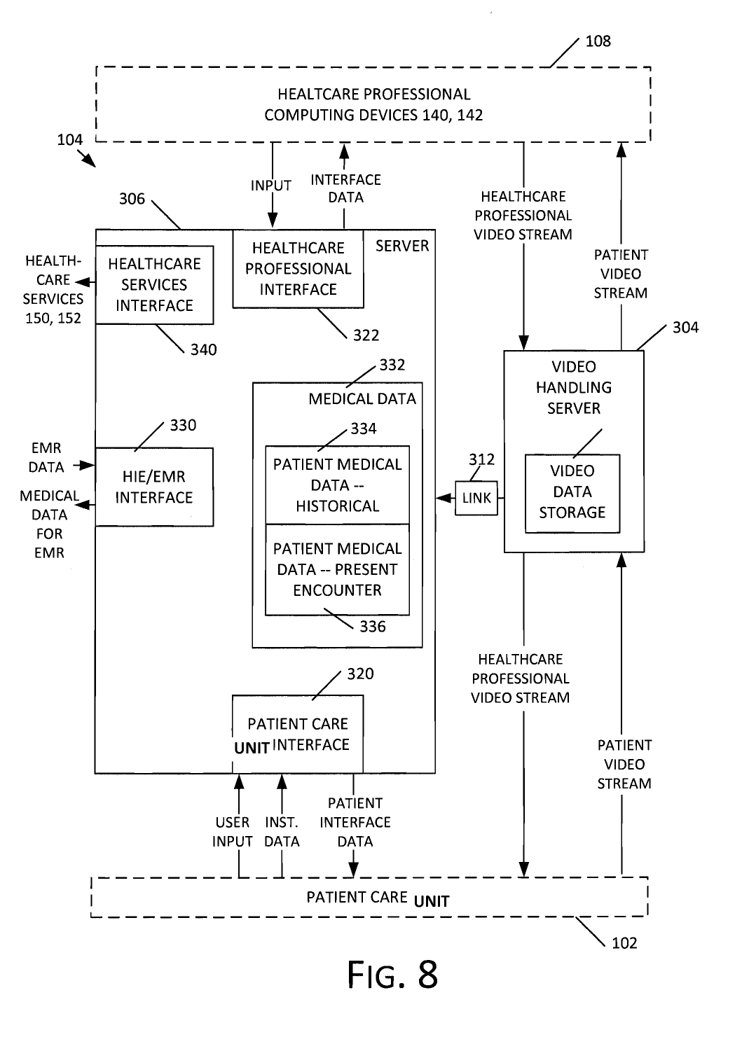

The care system server facilitates communication between the patient care unit 102 and the call center 108. For example, in some embodiments the care system server 104 controls the transmission of video streams between the patient's home H and the call center 108. In addition, in some embodiments the care system server 104 communicates data from the instruments 120 to the call center 108 where the data is presented to the healthcare professional H1, H2. An example of the care system server 104 is illustrated and described in more detail with reference to FIG. 8 herein.

Additionally, in some embodiments the care system server 104 communicates with a health information exchange (HIE) 106 to obtain historical patient medical data, such as from the patient's electronic medical record (EMR) system 130. The EMR system 130 stores the patient's electronic medical record, which contains historical patient medical data, such as information regarding past findings and diagnoses of healthcare professionals for the patient. In another possible embodiment the care system server 104 accesses the EMR system 130 without utilizing the health information exchange 106. Also, in some embodiments the care system server 104 sends current patient medical data to the HIE 106 and/or EMR 130 where it can be stored in the patient's medical records as historical patient medical data. In some embodiments, the HIE provides medical device data 132 (such as data relating to a patient's implantable medical device) or other data 134 to the care system server 104.

The call center 108 includes healthcare professionals H1, H2, such as nurses or physicians, which can interact with the patient P1 through the telemedicine care system 100. The call center 108 may be one or more physical locations, such as at a clinic or office building, and each physical location can include one or more healthcare professionals H1 and H2. Many healthcare professionals can be involved in providing care to different patients P at a given time.

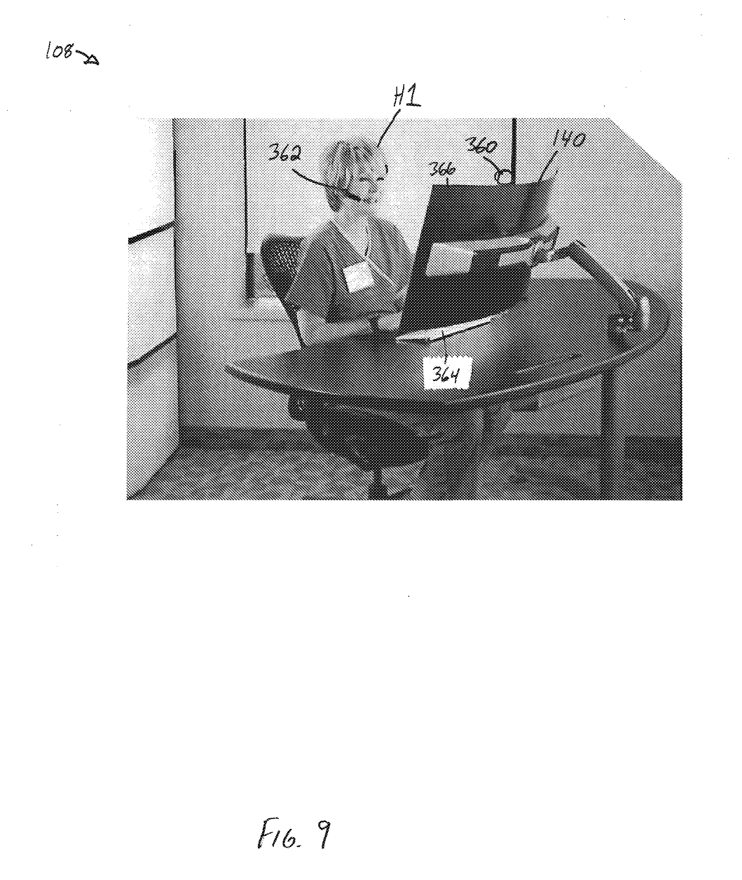

In some embodiments, the call center 108 includes healthcare professional computing devices 140, 142 that are used by the healthcare professionals H1, H2 to interact with patients P. The call center 108 is illustrated and described in more detail herein with reference to FIGS. 9-12.

In some embodiments, the call center 108 can communicate with other services, such as emergency care services 150 and hospital and other healthcare services 152. For example, if a healthcare professional H1, H2 determines that the patient P1 requires emergency care, the emergency care service 150 can be contacted by the healthcare professional H1, H2 to request the emergency care. As another example, if hospitalization or other specialized medical care 152 is required, such services 152 can be requested by the healthcare professional H1. These services 152 may involve scheduling an appointment for the patient P1 to be seen by the specialist, or may involve the healthcare professional H1 requesting that another healthcare professional H2 evaluate the patient P1. For example, if the healthcare professional H1 suspects that the patient may be having symptoms of a bladder infection, the healthcare professional H1 may submit a request to the care system server 104 to have the patient P1 evaluated by a urologist H2 through the telemedicine care system 100.

FIGS. 2-7 illustrate examples of the patient care unit 102.

FIG. 2 is a perspective diagram illustrating an exemplary environment in which the patient care unit 102 can be used. In this example, the patient care unit 102 is used within the home H of the patient P1. In this example, the patient P1 has an in-home care provider C1 (such as a parent) present. The patient care unit 102 includes the patient care device 103, the video camera 112, the microphone 114, the display 116, the speakers 118, and the instruments 120.

Either the patient or another care provider C1 can act as the primary communicator with the healthcare professional H1, H2 through the patient care unit 102. In the example illustrated in FIG. 2, the care provider C1 interacts with the healthcare professional H1, H2, such as to describe the symptoms that the patient P1 is experiencing. The healthcare professional H1, H2, or the patient care unit itself, may request that certain instruments 120 be used to collect data regarding the patient's current condition, and instructions on the proper use of such instruments are provided to the patient P1 or care provider C1. For example, the care provider C1 can place a blood pressure cuff on the patient's arm, and blood pressure data is communicated back to the patient care unit 102 for secure transmission to the computing device 140,142 of the healthcare professional H1, H2.

In some embodiments, the patient care unit 102 includes a video camera 112 that records and transmits a video stream of the patient P1 or care provider C1, so that the healthcare professional H1, H2 can see the person that he or she is interacting with. The video camera can be built into the patient care unit 102, such as illustrated in FIG. 2. In another possible embodiment, the video camera is an accessory, such as a headset or tripod mountable video camera. A headset can be worn by the care provider C1, for example, so that the healthcare professional H1, H2 can see the same things that are seen by the care provider C1, which can be particularly helpful to conduct a physical examination of the patient. A tripod mountable video camera can similarly be directed at the patient P1 to permit the healthcare professional H1, H2 to view the patient P1. In some embodiments, the patient care unit 102 includes multiple video cameras, such as one (or more) built-in video camera and one (or more) external video camera. The external video camera can communicate with the patient care device 103 using wireless (e.g., Wi-Fi (such as using any one of the IEEE 802.11 family of protocols), BLUETOOTH®, infrared, etc.) communication devices and protocols or wired communication devices and protocols.

Although the patient care unit 102 is illustrated as being a mobile unit that can be used within the patient's P1 home H, the patient care unit 102 can alternatively be used in a variety of other environments, such as within a workplace, an assisted living community, a business (including an office building or a daycare center), a retail store (including a pharmacy), a medical facility, a transportation vehicle such as a car, bus, boat, or airplane, an emergency vehicle, and the like. As an example, the patient care unit 102 can be used at a hospital or emergency room to assist with the preliminary intake and evaluation of patients. As another example, the patient care unit 102 can be used at a medical care facility to allow a remote healthcare professional to interact with the patient at the medical care facility. In some embodiments, the patient care unit 102 is a relatively immobile device, such as constructed as a kiosk.

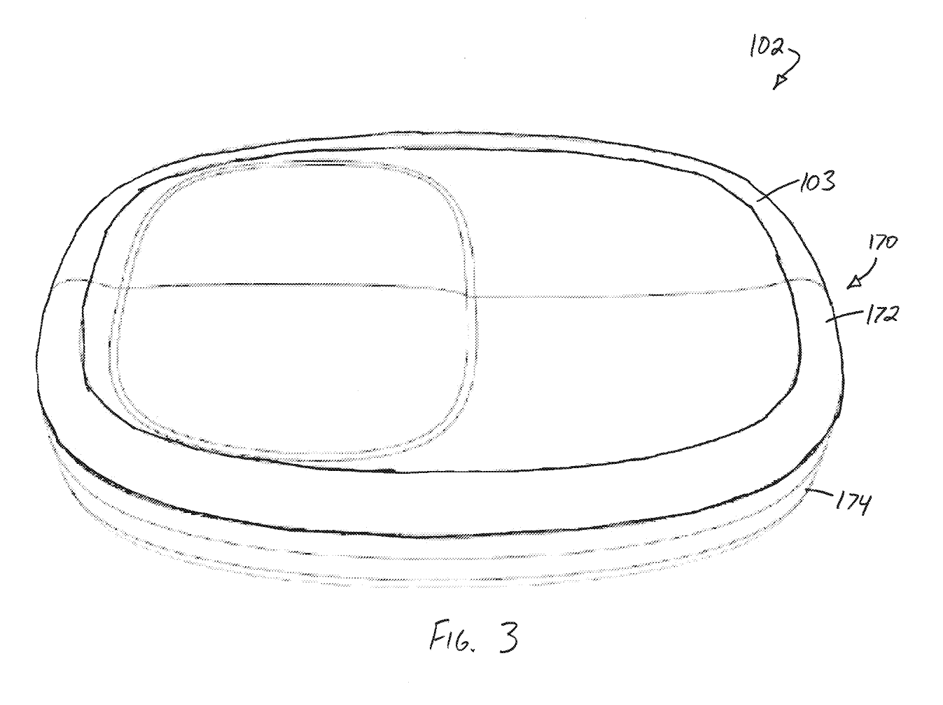

FIG. 3 is an isometric view illustrating the top and front of an example of the patient care unit 102 when arranged in a closed configuration. This example of the patient care unit 102 includes the patient care device 103 having a housing 170. The housing includes an upper housing 172 (display portion) forming a cover of the patient care device 103 and a lower housing 174 (base portion) forming a base of the patient care device 103. The upper and lower housings 172 and 174 are typically coupled together at the rear of the patient care device 103 by a hinge, not visible in FIG. 3, which permits the patient care device 103 to be opened to provide access to an interior compartment. The housing 170 is typically formed of an impact and water-resistant material, such as injection molded plastic or sheet metal, but can alternatively be formed of a variety of other materials. The housing 170 protects and encloses the interior components of the patient care unit 102.

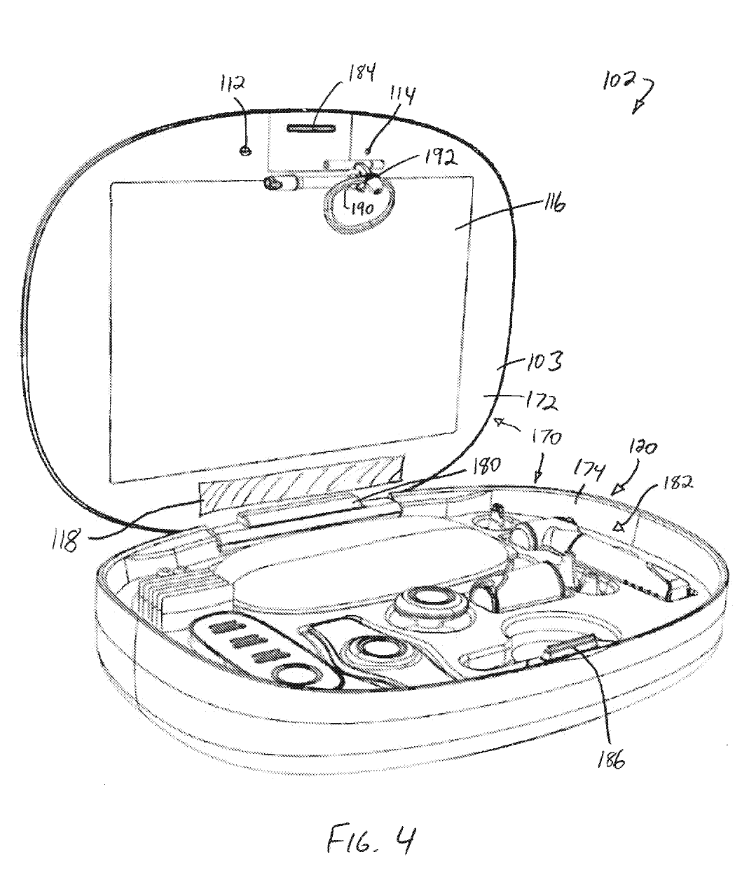

FIG. 4 is another isometric view of an example of the patient care unit 102 when arranged in an open configuration.

In this example, the patient care unit 102 includes the patient care device 103 and instruments 120. The patient care device 103 includes a housing 170, having an upper housing 172 and a lower housing 174 coupled together by a hinge 180. The upper and lower housings 172 and 174 define an interior space 182 that is configured to contain the instruments 120 when the patient care device 103 is in the closed configuration. A latch, including upper latch portion 184 and lower latch portion 186, supports the patient care device 103 in the closed configuration to reduce the chance that the patient care device 103 opens unintentionally.

In some embodiments, certain components of the patient care device 103 are contained at least partially within the upper housing 172, such as the video camera 112, the microphone 114, the display 116, and the speakers. Additional components (such as at least some of the internal electronics described herein) can be contained within the upper housing 172, although more or fewer components can be arranged within or supported by the upper housing 172 in other possible embodiments.

When the upper housing 172 is in the open configuration, the video camera 112 is positioned so that it is directed normal to the interior surface of the upper housing 172, and toward an anticipated location of the user.

In some embodiments, the upper housing 172 can support one or more instruments 120. In this example, a clip 190 extends from the upper housing 172 to support a headset 192. Other instruments can be similarly stored and supported by the upper housing 172 in other embodiments.

The lower housing 174 stores one or more instruments 120 within the interior space 182. Additional components, such as at least some of the internal electronics described herein, can also be contained within the lower housing. Heavier components, such as transformers or batteries, when included, will typically be contained within the lower housing to avoid having the upper compartment heavier than the lower compartment.

In some embodiments, the lower housing 174 includes built-in cradles. The cradles include a receptacle shaped to receive at least a portion of one of the instruments 120, to securely store the instrument in the lower housing 174 when not the instrument is not in use. In some embodiments, the cradles are charging cradles that operate to recharge batteries of the instruments 120 when the instruments 120 are in the charging cradle. In some embodiments the charging cradles include pins or other electrical conductors that align with and connect to corresponding conductors on the instruments to supply power to the instruments. In other embodiments, the charging cradles include non-contact chargers, which can wirelessly transfer power to the instruments, such as through inductive transfer of energy using an electromagnetic field.

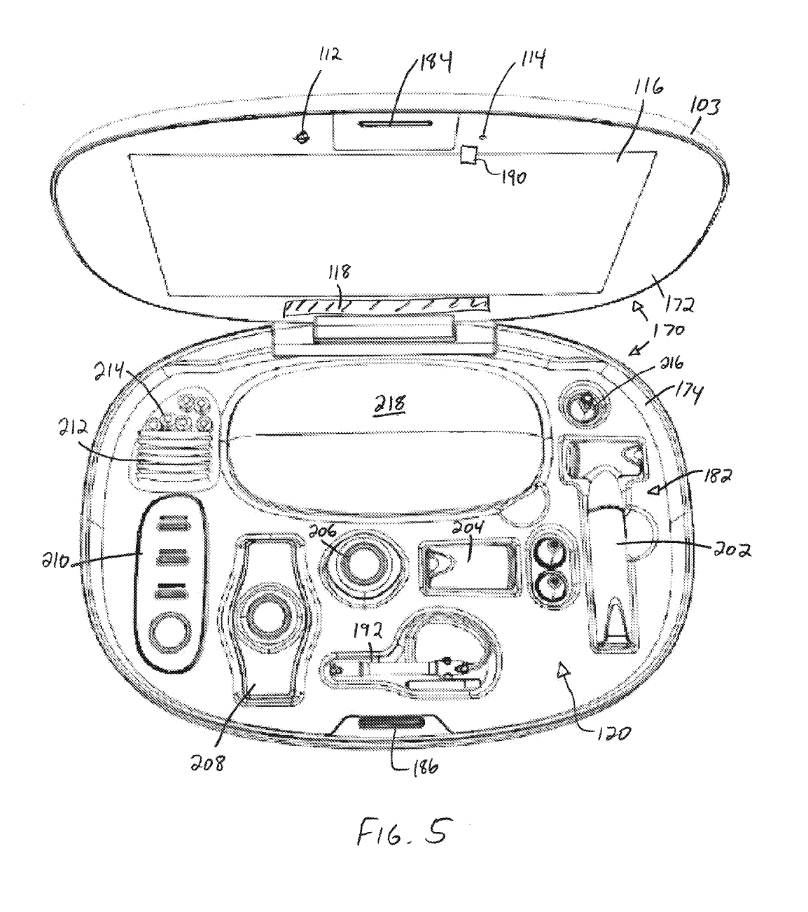

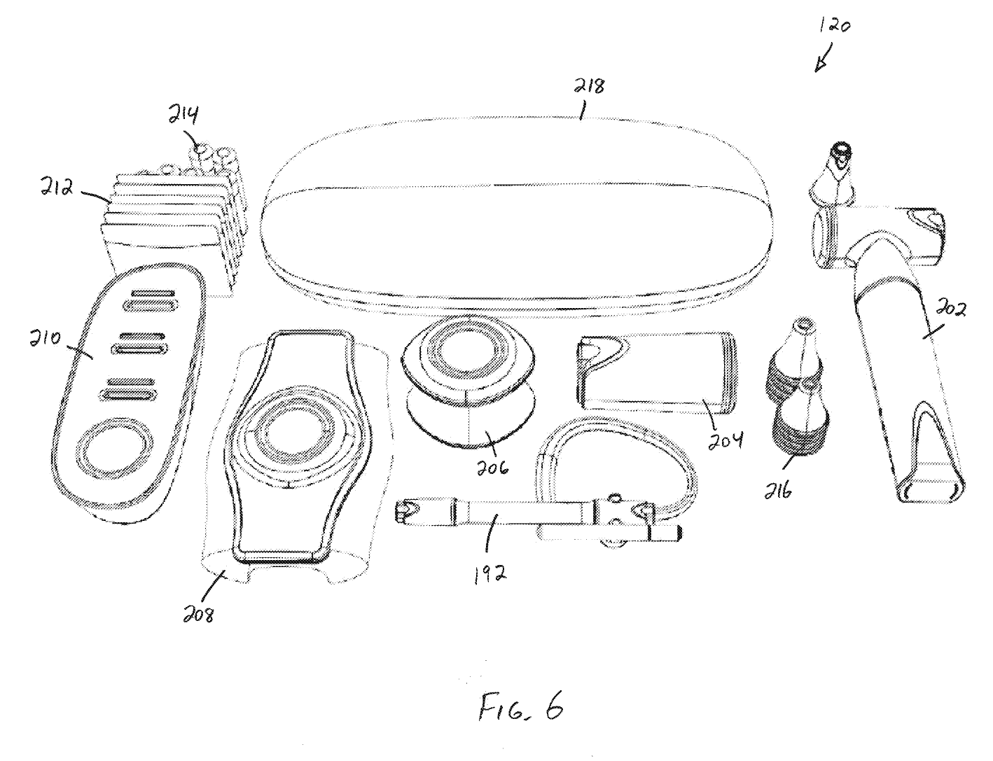

FIG. 5 is another isometric view of an example of the patient care unit 102. In this example, the lower housing 174 provides a storage area for a plurality of instruments 120 within the interior space 182. A variety of collections of different instruments 120 can be provided in various different embodiments of the patient care unit 102, such as depending on the intended purpose of the unit. The instruments 120 are described in more detail with reference to FIG. 6.

FIG. 6 provides a more detailed view of an exemplary collection of instruments 120 that can be included with the patient care unit 102. In this example, the instruments include the wireless headset 192, an otoscope 202, a pulse oximeter 204, a stethoscope 206, a blood pressure cuff 208, and a test device 210 and associated test strips 212. Additionally, the example unit 102 also provides batteries 214, replacement specula 216 for the otoscope 202, and a first aid kit 218.

The headset 192 provides enhanced communication capabilities and can be supported by the head of the patient. The headset 192 can include an ear attachment member that can be placed over the user's ear, or may be supported by a headband or strap. The headset 192 permits hands-free operation. The headset 192 includes one or more of: a microphone, a video camera, a speaker, and a light. In a wireless configuration, the headset 192 further includes a power source, such as a battery, and a wireless communication device. Audio and/or video signals or data are communicated from the headset 192 back to the patient care device 103 through wireless communication devices or through a wired connection to the patient care device 103. Similarly, audio signals or data can also be communicated from the patient care device 103 to the headset 192 where they are converted into audible sounds with the speaker. The headset is particularly useful when a care provider C1, other than the patient P1, is interacting with the patient care device 103, to permit the healthcare professional H1, H2 to see and hear the patient through the headset 192.

In another possible embodiment, a video camera (external to the patient care device 103) is provided that can be mounted on a tripod or placed on a table, and directed toward the patient, to aid the healthcare professional H1, H2 to better see the patient. The video camera may also include a microphone.

The otoscope 202 is provided in some embodiments to allow the healthcare professional to more carefully examine a patient, such as to view within the patient's ears, nose, or throat. In some embodiments, the images or video captured by the otoscope 202 are encoded in a digital form and sent to the computing device 140, 142 of the healthcare professional H1, H2 for examination by the healthcare professional H1, H2. Disposable specula 216 are provided in some embodiments. In some embodiments the otoscope includes a wireless communication device for communication with the patient care device 103.

A pulse oximeter 204 is provided in some embodiments to take a reading of the oxygenation of the patient's hemoglobin. Readings are transmitted via wire or wirelessly to the patient care device 103, for transmission to the computing device 140, 142 of the healthcare professional H1, H2.

A stethoscope 206 is provided in some embodiments for auscultation. The stethoscope 206 detects internal sounds within the body, such as the beating of the heart or breathing, and encodes and transfers the encoded sounds to the patient care device 103 for transmission to the computing device 140, 142 of the healthcare professional H1, H2, where the sounds can be heard by the healthcare professional H1, H2.

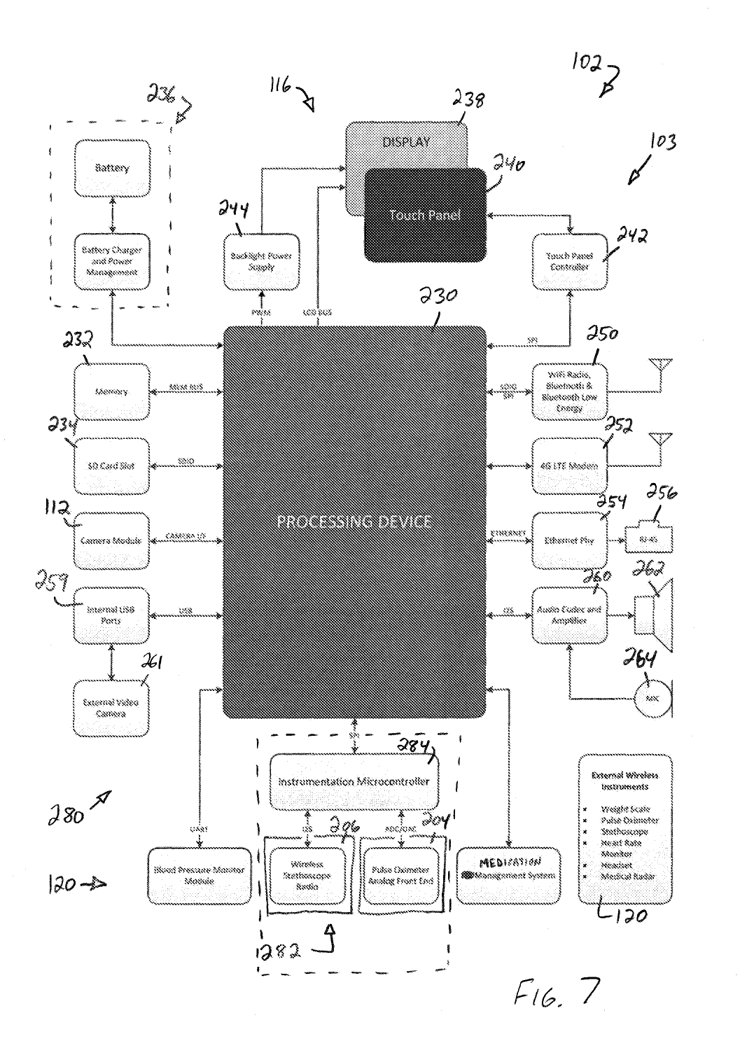

In some embodiments, the stethoscope 206 is a wireless stethoscope. As one example, the wireless stethoscope includes a housing and electronic components. The housing includes a stethoscope surface configured to receive sounds from a patient's body. The stethoscope surface can include a diaphragm or bell, for example. A microphone is arranged within the stethoscope 206 to convert the sounds into electronic signals that are detected and digitized. A processing device interacts with a wireless communication device to transmit the digital signals encoding the sounds to the patient care device 103, such as through the wireless communication device 250, shown in FIG. 7, or through a wireless stethoscope radio 206. The patient care device 103 then sends the data for delivery to the healthcare professional H1 or H2 where the sounds are played to the healthcare professional.

In some embodiments, the stethoscope surface is configured to removably attach to the patient's skin. The removable attachment can include an adhesive or other sticky surface, for example, or can involve a suction cup or light suction device (such as a vacuum). The attachment to the patient's skin reduces undesired noise that can otherwise be caused by movement of the stethoscope 206 on the patient's skin. Such noise could otherwise be confused by the healthcare professional as abnormal breath sounds, or irregular heart rhythms, for example.

A blood pressure cuff 208 is provided in some embodiments to measure the patient's P1 blood pressure. The blood pressure reading is sent to the patient care device 103 via wired or wireless communication and then transferred to the computing device 140, 142 of the healthcare professional H1, H2.

A test device 210 and associated test strips 212 are provided in some embodiments for performing tests on biological matter from the patient P1. In one embodiment, the test device 210 is a blood glucose monitor. In another possible embodiment, the test device 210 includes a lab-on-a-chip that can operate to perform a plurality of different biological tests on a given sample from the patient P1. Typically, the sample is provided onto a test strip 212, and then inserted into the test device 210. The test is then performed on the sample, and the results are provided to the patient care device 103, and transferred to the computing devices 140, 142 of the healthcare professional H1, H2 for display to the healthcare professional H1, H2.

Different embodiments of the patient care unit 102 can contain a variety of different collections of instruments 120. For example, in some embodiments the patient care unit 102 is configured to contain instruments relative to a particular disease or medical condition. Several examples of possible diseases or medical conditions include asthma, strep throat, ear infection, and heart failure. As a more specific example, a patient care unit 102 intended for a patient diagnosed with heart failure may include a collection of instruments including: a weight scale, a blood pressure cuff, a pulse oximeter, a wearable EKG device, and a video headset. As another example, a patient care unit 102 intended for a patient diagnosed with asthma may include a spirometer/flow meter, a blood pressure cuff, a stethoscope, an otoscope, and a pulse oximeter. Other embodiments of the patient care unit 102 include other collections of one or more instruments 120.

Another example of a possible instrument is an inter-cardiac pressure detection device. One example of an intracardiac pressure detection device is a device that utilizes radar technology to detect and determine intracardiac pressures.

Another example of a possible instrument is a pulmonary edema detection device. One example of a pulmonary edema detection device is a device that utilizes radar technology to detect lung behaviors consistent with left ventricle failure.

In some embodiments, at least some of the instruments 120 are integrated with the patient care device 103, such that at least one of the electronic components of the integrated instruments 120 are contained within the housing of the patient care device 103. In some embodiments, integrated instruments 120 are inoperable for the intended purpose if not used with the patient care device 103. One of the advantages of using integrated instruments 120, in some embodiments, is that certain components can be shared by multiple instruments, which may reduce the cost and complexity of the patient care unit 102. For example, a single wireless communication device can be used to communicate with multiple different wireless instruments. As another example, a single instrument processing device 284 can be used to process data from and communicate with multiple instruments 120. Integrated instruments 120 may also include one or more components that are contained within the housing of the patient care device, rather than being contained in the instrument 120 housing or external from the patient care device 103. The integrated instruments 120 can share a common power supply or battery charger and power management system. Examples of integrated instruments 120 are described herein. Some specific examples include an integrated blood pressure monitor cuff, an integrated pulse oximeter, and an integrated wireless stethoscope.

In some embodiments, the patient care unit 102 communicates with one or more external instruments that are not integrated with the patient care device 103. Examples are described herein. Another example is an external dialysis instrument.

FIG. 7 is a schematic block diagram illustrating an example of the electronic components of the patient care unit 102. The patient care unit 102 includes the patient care device 103, and one or more instruments 120, such as a pulse oximeter 204 and a wireless stethoscope 206.

In some embodiments, the patient care device 103 includes a processing device 230, memory 232 and 234, power management circuitry 236, a display 116, and instrument and other input/output interfaces.

The processing device 230 is a device capable of executing data instructions, which can be stored in a computer readable storage device, such as the memory 232 or 234. An example of a processing device 230 is an application processor, such as the Texas Instruments Sitara model AM3874, or the Texas Instruments DaVinci model DM3730. The application processor 230 is suitable to handle the processing of the multiple possible simultaneous video streams, while continuing to perform additional processing necessary for communication with instruments and generation of the patient care device 103 user interface.

Any suitable memory devices can be used. As one example, memory 232 can be provided through a memory socket, such as a SODIMM, or memory devices directly on the circuit board. Non-volatile memory 234 can be provided in some embodiments through an SD card slot. In some embodiments, the SD card slot 234 is not accessible from outside of the housing.

Power management circuitry 236 provides appropriate power to the electronic components. In some embodiments, the power management circuitry 236 includes one or more power sources, such as a battery. Some embodiments include a rechargeable battery. Examples of batteries include Lithium batteries and Lithium ion batteries. An external power source can also or alternatively be used, such as an external AC power adapter to receive power from a wall socket. In some embodiments the power management circuitry 236 includes a battery charger, which operates to recharge the battery from an external power source, such as when connected to a wall receptacle.

The display 116 is provided to display a graphical user interface for interaction with the user. One example of a suitable display is a thin-film transistor liquid crystal display (TFT LCD) 238. In some embodiments, the display 116 is a touch-sensitive display, such as including a capacitive or resistive touch panel 240 and touch panel controller 242 operable to detect inputs provided by the user onto the display 116. Some embodiments include a backlight power supply to provide adequate power for the display 116.

Some embodiments include one or more cameras, such as the build-in front facing video camera 112. In some embodiments, the video camera is of a type capable of 720p recording. Other embodiments include other resolutions.

Additional instrumentation and input/output interfaces can also be provided in various embodiments. For example, in some embodiments the patient care unit 102 includes a wireless communication device 250. Examples of a wireless communication device include a Wi-Fi radio transceiver (such as a device that transmits and receives data according to a standard Wi-Fi data communication protocol, such as any one of the IEEE 802.11 family of wireless communication protocols), a BLUETOOTH® and BLUETOOTH® low energy radio transceiver), a wired data communication device 254 (such as an Ethernet physical layer device and associated Ethernet port 256 (RJ-45)), a cellular network communication device (e.g., a 3G, 4G, or 4G LTE modem) 258, and internal USB ports 259. The USB port 259 can be utilized to connect with instruments or other electronic devices. For example, an external video camera 261 can be connected by a USB cable connected to the internal USB port 259 (or an externally accessible USB port).

Some embodiments include both a Wi-Fi radio transceiver and a Bluetooth radio transceiver. In some embodiments, the Wi-Fi radio transceiver and the Bluetooth radio are both part of a single wireless communication device, while in other embodiments they are separate devices. The Wi-Fi and Bluetooth transceivers can be used to communicate with Wi-Fi or Bluetooth enabled instruments 120 having suitable wireless communication devices. The wireless communication device 250, wired communication device 254, and cellular network communication device 258 can also be used to communicate across a data communication network 110, such as to transmit and receive data from the care system server 104 and the healthcare professional H1, H2 computing devices 140 and 142 (shown in FIG. 1). Although only one network communication device can be provided in some embodiments, multiple different network communication devices provide greater flexibility. For example, the cellular data communication device may be able to communicate at times and locations where WiFi or Ethernet communication are not readily available, and vice versa. Some embodiments include a Wi-Fi transceiver, a Bluetooth transceiver, a cellular network communication device 258, and a wired communication device 254. In some embodiments, the patient management device can communicate with a satellite radio to communicate across a satellite communication network when other forms of communication may not be available (such as in a remote location or on a watercraft). In some embodiments, the patient care unit 102 is used to provide concierge-type medical services by connecting a patient with a remote healthcare professional.

Some embodiments include an audio codec and amplifier device 260, which is coupled to a speaker 262 and/or a microphone 264. The speaker 262 generates audible sounds, such as the voice of the healthcare professional H1, H2, or sounds or instructions provided by the patient care device 103. The microphone 264 operates to detect sounds generated by the patient P1 or associated care provider C1.

When designing and building devices involved with medical technology—particularly within the United States, governmental regulations are important considerations, which can have a large impact on the cost of the devices (in which a lower cost is preferred) and the time to market (in which a faster time to market is preferred).

Under U.S. regulatory practices, medical devices are typically categorized within one of three classes ranging from class 1—which are the least regulated, to class 3—which are most closely regulated. As a result, regulatory expenses and time to market will often be lower for a medical device that is classified in a lower class (e.g., 1) than a medical device that is classified in a higher class (e.g., 3).

Certain medical devices that are involved in the diagnosis and treatment of a patient are classified as class 2 medical devices. Accordingly, some of the instruments of the patient care unit 102 may be classified as class 2 medical devices. However, another device that interacts with or is sufficiently involved with another class 2 medical device can be considered an accessory to a class 2 medical device, requiring that that device also be regulated under the class 2 standards. Accordingly, if the patient care device 103 is sufficiently involved in the processing of data, for example, from one or more instruments 120, it is possible that the patient care device 103 would also be considered a class 2 medical device. A particular drawback to this is that any changes in the design of the patient care device 103, even a minor change to the software, may require resubmission and reevaluation by the Food and Drug Administration, thereby increasing the regulatory costs and delaying the time to market for the updated design.

In an effort to reduce or eliminate these problems, some embodiments of the patient care device 103 include a bifurcated design, including a first portion 280 and a second portion 282. The first portion 280 includes the main processing device 230 and performs the primary the processing, control, and communication functions of the patient care device 103, but does not perform any data processing or encryption of instrument 120 data, other than to store the data or pass the data on to another device (such as the care system server 104) through one of the communication devices. The second portion 282 includes a second instrument processing device 284 that is separate from but in data communication with the main processing device 230. The second processing device 284 is used to perform data processing, encryption, or other functions in cooperation with one or more instruments 120, when such functions may be subject to heightened regulatory scrutiny. In this way, a revision or modification made to the first portion can be made without requiring changes to be made to the second portion 282, which may reduce or eliminate the need for additional regulatory review or approval of the patient care device 103 to implement the revision or modification to the first portion 280.

In this example, the instrument processing device 284 interacts with a wireless stethoscope 206 through wireless communication with a wireless stethoscope radio communication device 290, and also with a pulse oximeter through the pulse oximeter analog front end 292. More, fewer, or different instruments can be configured to interact with the instrument processing device 284 in other embodiments. Further, in some embodiments the patient care device 103 includes a separate instrument processing device 284 for each of one or more of the instruments, such that multiple instrument processing devices 284 are included in some embodiments.

Some embodiments of the patient care device 103 do not include separate first and second portions 280 and 282.

Additional or different instruments 120 are included in some embodiments. Examples of other possible instruments include a blood pressure monitor module, a thermometer (such as an oral thermometer, an ear temperature thermometer, or a temporal scanner thermometer), a digital spirometer, a wireless weight scale, a wireless pulse oximeter, a wireless stethoscope, a wireless heart rate monitor, a wireless headset, and wireless medical radar. The wireless devices can either communicate through a wireless interface associated with the instrument processing device 284 (e.g., the radio 206 or similar radio), or through the wireless communication device 250, such as using Wi-Fi or Bluetooth.

In some embodiments, the patient care unit 102 includes a global positioning system device, or other location identification device. Location data can be used, for example, to identify a location of the patient if emergency care is needed, or to provide the patient with location-specific information, such as a nearby pharmacy, care facility, fitness center, and the like.

Some embodiments of the patient care unit 102 include an air quality detection device. The air quality detection device operates to detect various characteristics of the air at the location where the patient care unit 102 is operating. For example, the air quality detection device can detect one or more allergens or other triggers for medical conditions, such as asthma. Several examples include pollen, mold, and smoke. Examples of air quality detection devices include a smoke detector, and a lab-on-a-chip.

FIG. 8 is a schematic block diagram of an exemplary configuration of the care system server 104, in which video and non-video data communications are handled by separate servers which provide separate data communication paths. However, in another possible embodiment all data communication can be handled by a single server or distributed across a set of servers, or a different division of data communication can be applied across multiple servers. In yet another possible embodiment, data communication can be provided directly between the patient care unit 102 and the healthcare professional H1, H2 computing devices 140, 142, or by utilizing additional intermediary computing devices.

In this example, the care system servers 104 include a video-handling server 304 and a non-video handling server 306.

The video-handling server 304 can include one or more server computing devices that receive video feeds from one or more of the patient care unit 102 and the healthcare professional computing device H1, H2, and pass the video feeds to the other party. In addition, in some embodiments the video-handling server 304 stores a copy of the video recording in video data storage 310. In some embodiments, the video-handling server 304 generates a link 312 (e.g., a URL) to a web page where the video of the patient interaction can be viewed from the video data storage 310. In some embodiments the video feeds include audio, while in other embodiments audio is separate from the video. The link 312 can be provided to the non-video handling server 306 for inclusion within a report and storage within the patient's electronic medical record, such that the video can be subsequently reviewed by a healthcare professional. An example of a system suitable for implementing the video-handling server 304 is the LinkLive system available from Revation, Inc. of Bloomington, Minn.

The non-video handling server 306 includes one or more computing devices that handle data communication of non-video data, as well as performs additional functions, such as interfacing with a health information exchange and/or electronic medical records system. In some embodiments, the server 306 is or includes a web server.

In this example, the server 306 includes a patient care unit interface 320 and a healthcare professional interface 322.

The patient care unit interface 320 receives data communications from the patient care unit 102, which may include user input data and instrument data. The user input data can include commands or other inputs received from the user through the patient care unit 102, such as through the touch-sensitive display 116 or another input device. The instrument data is data from one or more of the instruments 120, such as including data regarding the patient's current medical condition. For example, the instrument data can include a blood pressure reading from the blood pressure cuff 208.

The patient care unit interface 320 can also provide patient interface data to the patient care unit 102. The patient interface data can include data from the healthcare professional, medical data, instrument data, instructions on the use of the patient care unit, commands to be executed by the patient care unit 102, and the like. In another possible embodiment, the patient care unit interface 320 is a web server that generates and sends web page data to the patient care unit 102. The patient care unit 102 provides a graphical user interface that is displayed by the patient care unit 102 through a browser software application. In some embodiments the patient care unit 102 executes a local software application that generates the user interface, and the local software application sends and receives data through one or more of servers 304 and 306. An advantage of having at least some of the operations performed by a local software application is that the user interface can be generated even at times when data communication and/or one or more of the servers 304 and 306 are unavailable.

The healthcare professional interface 322 operates to communicate with the healthcare professional computing devices 140, 142. In some embodiments, the healthcare professional interface 322 operates as a web server that generates and sends healthcare professional interface data in the form of web pages that can be displayed by the healthcare computing device 140, 142 through a browser software application. The web server communicates using standard Internet Protocol communications, and can encode the web page data utilizing standard data communication standards, such as hypertext markup language (HTML). The healthcare professional interface 322 also receives healthcare professional input data from the healthcare professional computing devices 140, 142. For example, the web pages can contain text fields and selectable controls (such as buttons, menus, selectable graphical elements, and the like) through which the healthcare professional H1, H2 can provide inputs that are communicated back to the healthcare professional interface 322. In another possible embodiment, the healthcare professional computing device 140, 142 executes a local software application that performs at least some of these functions, and receives data sent to it from the healthcare professional interface 322.

In some embodiments, the server 306 operates to communicate data between a health information exchange and/or electronic medical record system. In this example, the server 306 includes an HIE/EMR interface 330 that performs the communications. For example, when a connection is made between the server 306 and a patient care unit 102 for the patient P1, the HIE/EMR interface 330 sends a request for electronic medical records associated with the patient P1. The electronic medical records data is provided to the server 306 in response to the request. In some embodiments, the electronic medical records data is stored by the server 306 as medical data 332 in a computer readable storage device. More specifically, the electronic medical records data is stored as historical patient medical data 334. The historical patient medical data 334 can then be provided to the healthcare professional computing device 140, 142 by the healthcare professional interface 322, for display to and review by the healthcare professional H1, H2. This permits the healthcare professional H1, H2 to obtain a much more complete understanding of the patient's past and current medical conditions, including previously documented findings, past and current treatments, such as past and current prescribed medications, and the like.

As instrument data is received from the patient care unit 102 by the patient care unit interface 320, such data can also be stored in the medical data 332. For example, the data is stored as current patient medical data 336. This information can also be provided to the healthcare professional computing device 140, 142 by the healthcare professional interface 322, where it can be displayed to and reviewed by the healthcare professional H1, H2. In some embodiments, the instrument data is also provided back to the patient care unit 102 from the server 306 for display to the patient. In some embodiments the healthcare professional H1, H2 must provide an input confirming or approving of the data before the data is sent back to the patient care unit 102 or otherwise made available for display to the patient P1 or care provider C1.

In some embodiments, the server 306 includes a healthcare services interface 340. The healthcare services interface 340 communicates with other healthcare services and professionals that may be needed to care for the patient P1. One example of a healthcare service is an emergency care service 150 (shown in FIG. 1). The healthcare services interface 340 permits the healthcare provider H1, H2 or the patient P1 or care provider C1 to communicate with an emergency dispatcher to request emergency care 150 be sent to the location of the patient P1. In another possible embodiment, the healthcare services interface can be used to schedule appointments with other healthcare professionals, submit prescriptions to a pharmacy, request laboratory tests, or otherwise communicate requests for additional information or healthcare services.

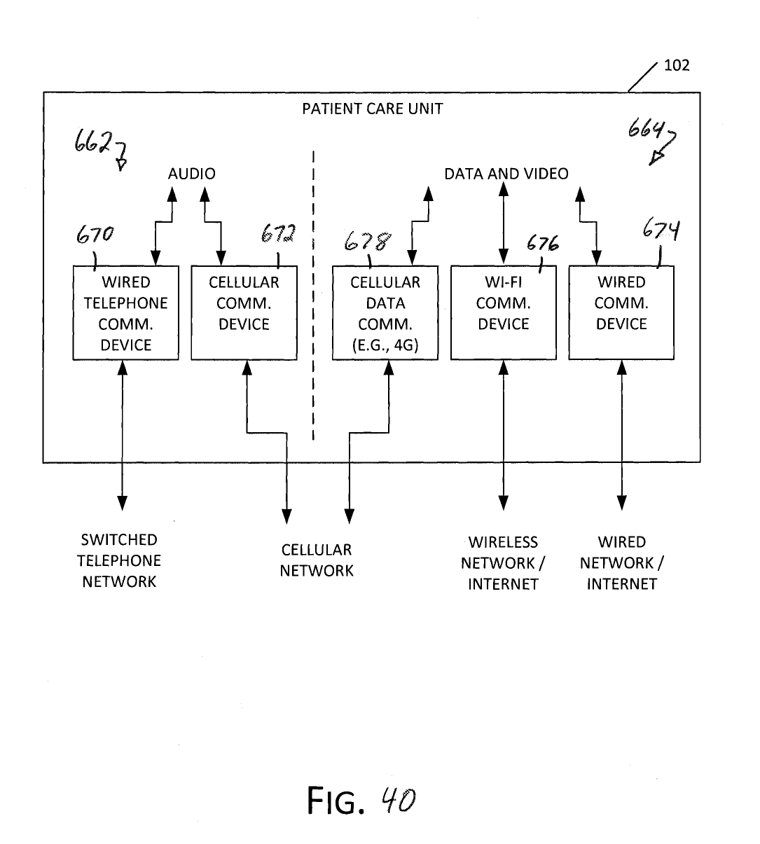

In some embodiments, audio (e.g., one or more voice channels) is communicated between the healthcare professional computing device 108 and the patient care unit 102 separate from the data and video. An example is illustrated in more detail with reference to FIG. 40. For example, the voice channel can be separately communicated across a switched telephone network or across a cellular telephone network. A benefit of separating the audio from other data, and communicating it across a reliable communication network, is that it reduces latency of the voice communications that may otherwise occur.

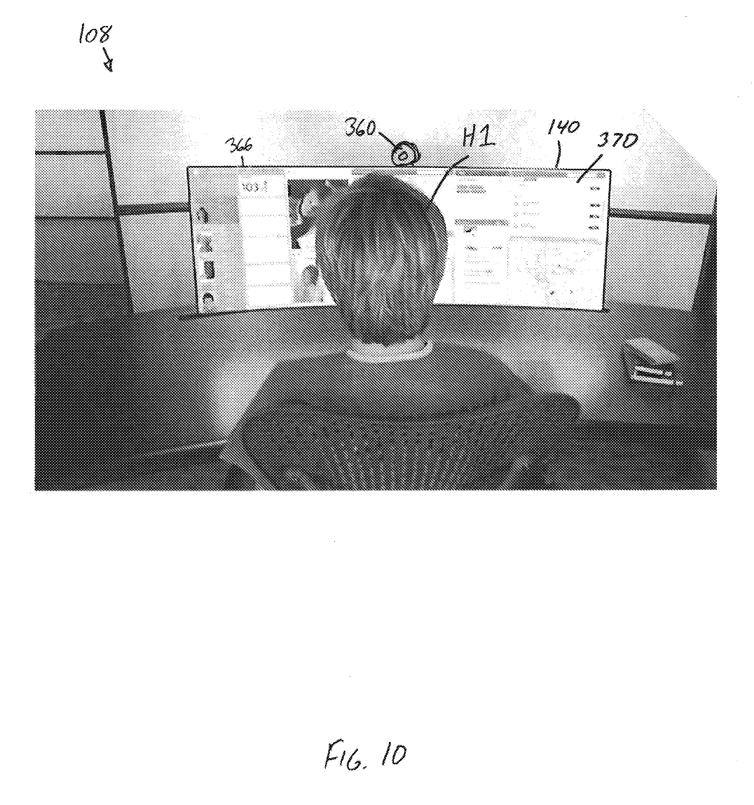

FIGS. 9 and 10 illustrate an exemplary station of a call center 108. The station includes a healthcare professional H1, and a healthcare professional computing device 140. The computing device 140 typically includes one or more input devices, such as the video camera 360, headset microphone 362, and a keyboard 364 shown in FIG. 9. The computing device 140 also typically includes one or more output devices, such as the headset speaker 362 and display device 366, also shown in FIG. 9. Other input and output devices can be used in other embodiments.

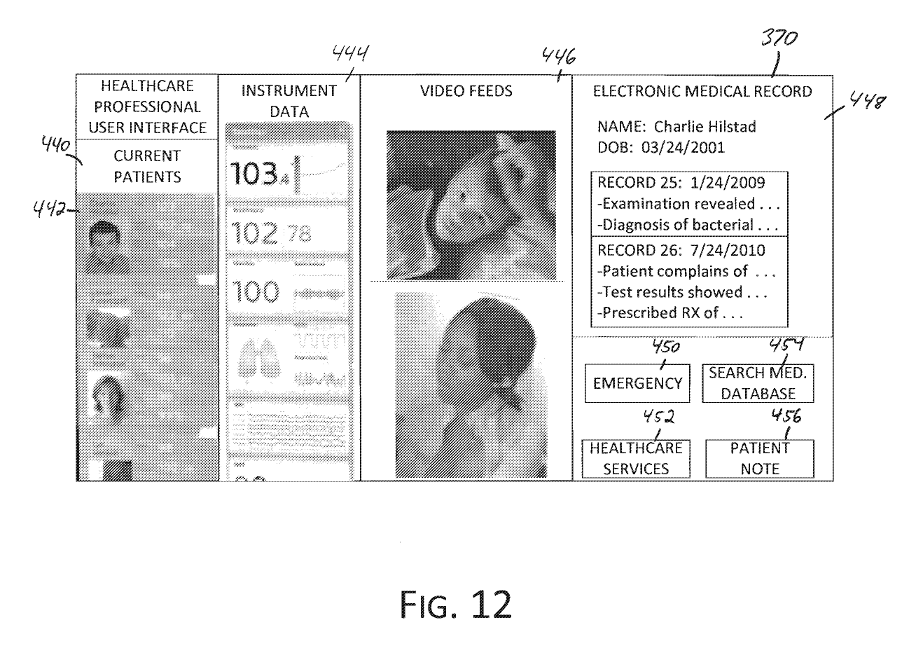

A user interface 370 is presented to the healthcare professional H1 by the display device 366. An example of the user interface 370 is described in more detail herein with reference to FIG. 12.

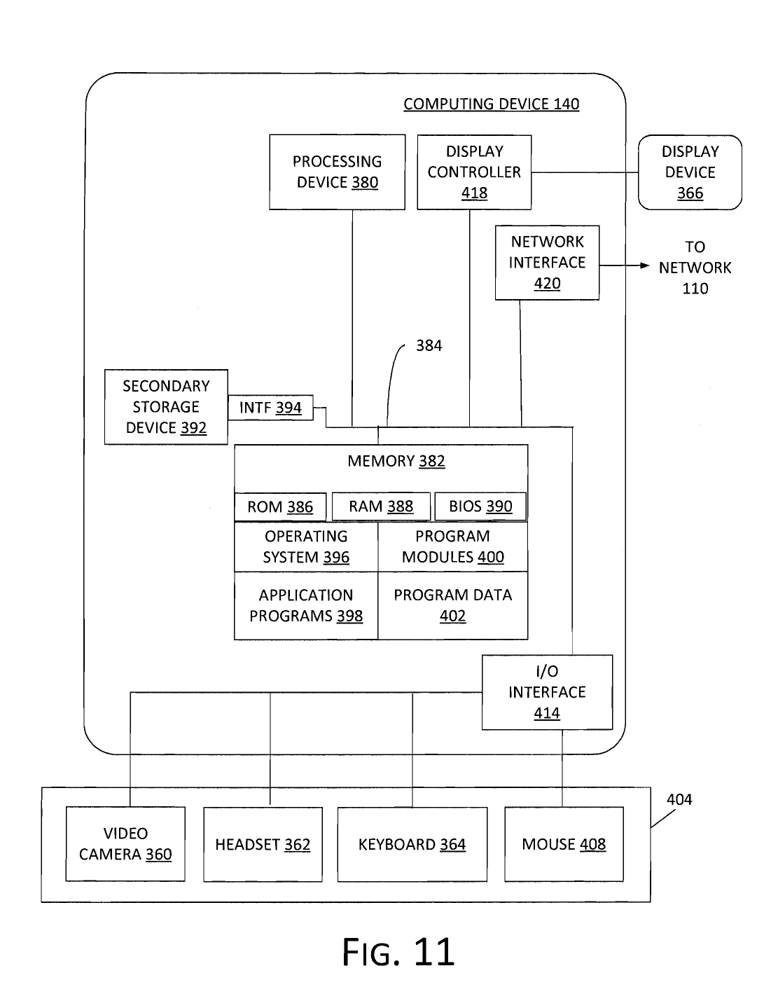

FIG. 11 illustrates an exemplary architecture of a computing device that can be used to implement the healthcare professional computing device 140. The same or similar computing device architecture can be used to implement other computing devices described herein, such as any one of the server computing devices 104, 304, 306; other healthcare professional computing devices 142; healthcare services 150, 152 computing devices; HIE/EMR servers; and the like. Further in some embodiments the patient care unit 102 includes a computing device architecture as illustrated and described with reference to FIG. 11. Accordingly, such computing device architectures will not be separately described herein in further detail to avoid undue repetition. The computing device can be used to execute the operating system, application programs, and software modules and engines described herein.

The computing device 140 includes, in some embodiments, at least one processing device 380, such as a central processing unit (CPU). A variety of processing devices are available from a variety of manufacturers, for example, Intel or Advanced Micro Devices. In this example, the computing device 140 also includes a system memory 382, and a system bus 384 that couples various system components including the system memory 382 to the processing device 380. The system bus 384 is one of any number of types of bus structures including a memory bus, or memory controller; a peripheral bus; and a local bus using any of a variety of bus architectures.

Examples of computing devices suitable for the computing device 140 include a desktop computer, a laptop computer, a mobile computing device (such as a smart phone, tablet computer (such as the iPad® mobile digital device or the Samsung Galaxy Android tablet), an iPod® mobile digital device, or other mobile devices), or other devices configured to process digital instructions.

The system memory 382 includes read only memory 386 and random access memory 388. A basic input/output system 390 containing the basic routines that act to transfer information within computing device 140, such as during start up, is typically stored in the read only memory 386.

The computing device 140 also includes a secondary storage device 392 in some embodiments, such as a hard disk drive, for storing digital data. The secondary storage device 392 is connected to the system bus 384 by a secondary storage interface 394. The secondary storage devices 392 and their associated computer readable media provide nonvolatile storage of computer readable instructions (including application programs and program modules), data structures, and other data for the computing device 140.

Although the exemplary environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media.

A number of program modules can be stored in secondary storage device 392 or memory 382, including an operating system 396, one or more application programs 398, other program modules 400 (such as the software engines described herein), and program data 402. The computing device 140 can utilize any suitable operating system, such as Microsoft Windows™, Google Chrome™, Apple OS, and any other operating system suitable for a computing device. Other examples can include Microsoft, Google, or Apple operating systems, or any other suitable operating system used in tablet computing devices.

In some embodiments, a user provides inputs to the computing device 140 through one or more input devices 404. Examples of input devices 404 include a video camera 360, headset 362, keyboard 364, and mouse 408. Other embodiments include other input devices 404. The input devices are often connected to the processing device 380 through an input/output interface 414 that is coupled to the system bus 384. These input devices 404 can be connected by any number of input/output interfaces, such as a parallel port, serial port, game port, or a universal serial bus. Wireless communication between input devices and the interface 414 is possible as well, and includes infrared, BLUETOOTH® wireless technology, 802.11a/b/g/n, cellular, or other radio frequency communication systems in some possible embodiments.

In this example embodiment, a display device 366, such as a monitor, liquid crystal display device, projector, or touch sensitive display device, is also connected to the system bus 384 via an interface, such as a display controller 418. In addition to the display device 416, the computing device 140 can include various other peripheral devices (not shown), such as speakers or a printer.

When used in a local area networking environment or a wide area networking environment (such as the Internet), the computing device 140 is typically connected to the network 110 through a network interface, such as an Ethernet interface 420. Other possible embodiments use other communication devices. For example, some embodiments of the computing device 140 include a modem for communicating across the network.

The computing device 140 typically includes at least some form of computer readable media. Computer readable media includes any available media that can be accessed by the computing device 140. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 140.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 11 is also an example of programmable electronics, which may include one or more such computing devices, and when multiple computing devices are included, such computing devices can be coupled together with a suitable data communication network so as to collectively perform the various functions, methods, or operations disclosed herein.

FIG. 12 is a screen shot illustrating an example of the healthcare professional user interface 370. In this example, the user interface 370 includes a listing 440 of patients being assisted or monitored by the healthcare professional H1 including a current patient identifier 442. The user interface 370 further includes a display 444 of instrument data (such as generated by the instruments 120), a display 446 of video feeds from the patient care unit 102 video cameras, and electronic medical record data display 448. The user interface further includes selectable controls that can be selected by the healthcare provider to initiate additional functions. The selectable controls include, for example, an emergency button 450 to request emergency care for the patient, a healthcare services button 452 to request or access additional healthcare services, a search medical database button 454 to access a database of medical data, and a patient note button 456 to initiate the documentation of the current encounter for entry into the patient's electronic medical record.

FIGS. 13-33 are screen shots illustrating various aspects of an exemplary user interface of the patient care unit 102, and additional features and functions that can be provided by some embodiments of the patient care unit 102.

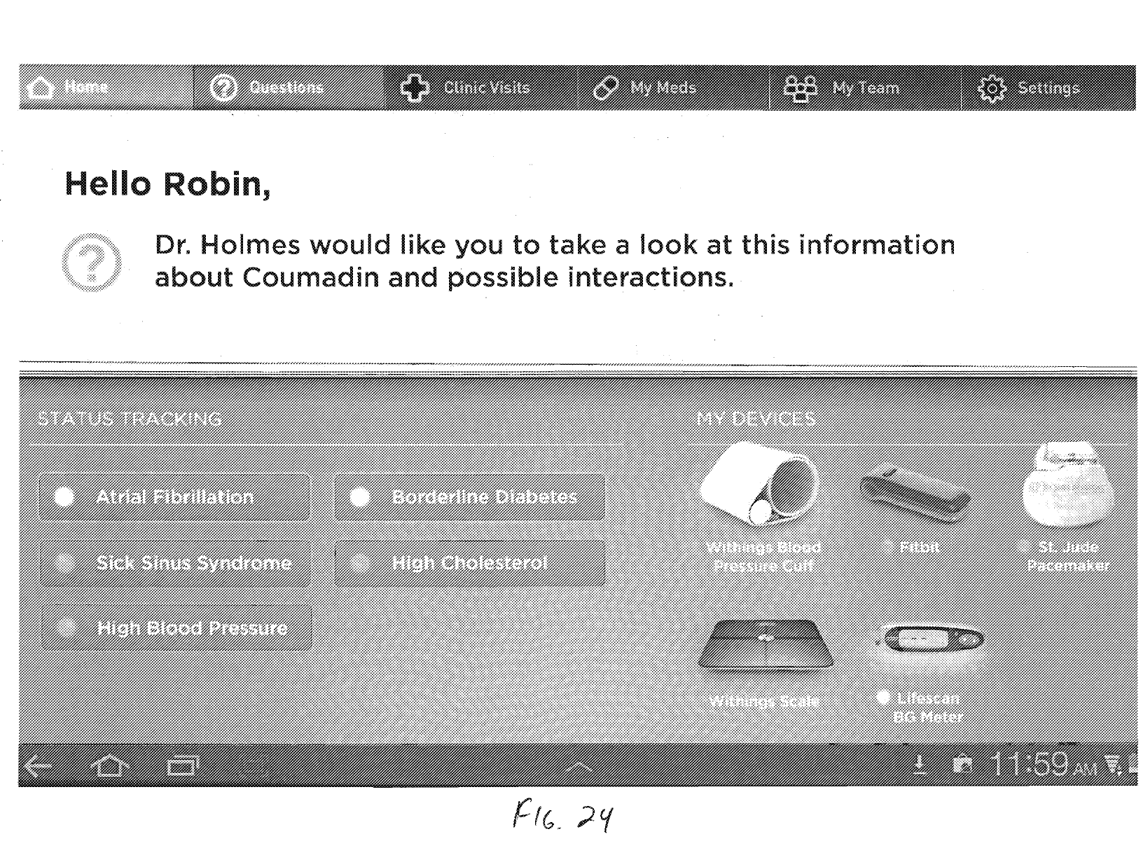

For example, in some embodiments the patient care unit 102 is or includes a medication management system. Screen shots illustrating exemplary features of the medication management system are shown in FIGS. 24-27 and 30-33.

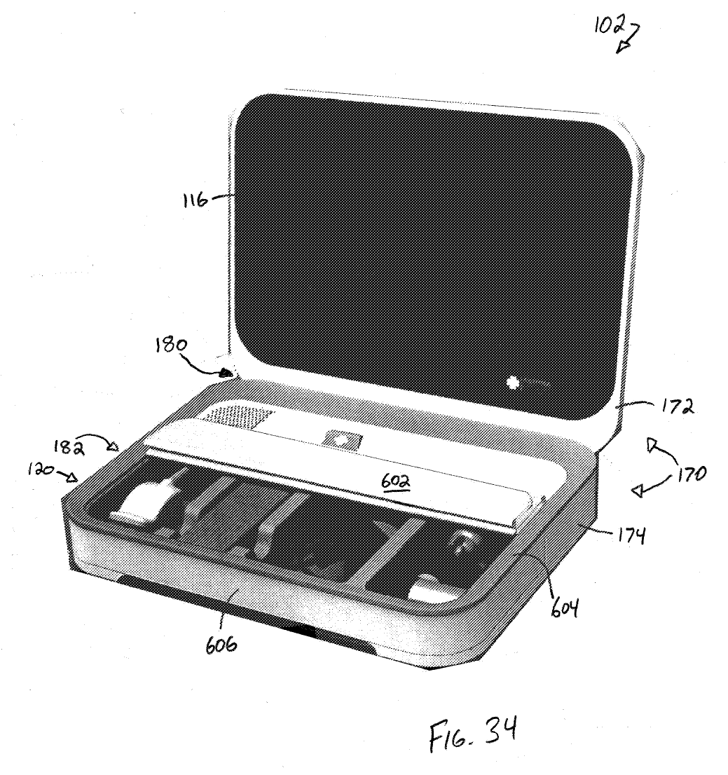

FIG. 34 is a perspective view of another example of the patient care unit 102. The example patient care unit 102 shown in FIG. 34 shares many common features of the example patient care unit 102 shown and described with reference to FIGS. 4-6, and therefore many of the common features will not be repeated here. For example, the patient care unit 102 includes a housing 170 including an upper housing 172 and a lower housing 174 connected by a hinge 180.

The housing has a closed position and an open position (as shown in FIG. 34). When in the closed position, the housing 170 includes an interior space 182 for storing one or more instruments 120. In this example, the interior space includes a recessed region of the lower housing 174. The housing 170 also has an open position, in which the upper housing 172 is pivoted about the hinge 180, such that the upper housing 172 extends generally vertically upward from the lower housing 174, to provide access to the interior space 182 and make the display arranged on a surface of the upper housing 172 visible to the user.

In some embodiments, the housing further includes a removable cover 602. The cover 602 is configured to be disposed across an upper portion of the lower housing 174. The cover 602 can be made of materials such as plastic, card board, wood, metal, leather, or combinations of these or other materials, and is designed to protect the display 116. For example, if one of the instruments 120 is not properly inserted into the interior space 182, the instrument 120 may extend out from the top of the interior space. When the cover is placed across the instruments 120 and the interior space 182, it will become apparent to the user that the instrument 120 is not properly inserted because the cover 602 will not lie flat. This alerts the user that an instrument 120 is misplaced, and allows the user to correctly insert the instrument 120 before closing the upper housing 172 onto the interior space. Even if the user does not notice the improper insertion, and proceeds to attempt to close the upper housing 172, the cover 602 acts to protect the display 116 and instrument 120 from damage. For example, the cover 602 distributes the closing force applied to the display 116 and misplaced instrument 120 across a larger area of the display 116, reducing the chance that the display 116 or instrument 120 will be damaged.

The cover 602 is removable, or at least openable, to permit access to instruments 120 contained within the interior space 182. In this example, the cover 602 is permanently connected to the lower housing at one end, and includes a plurality of folds. The cover 602 can be lifted at an opposite end, and folded along the folds for temporary storage in a folded configuration (shown in FIG. 34) while the instruments 120 are in use. After use, the cover 602 can then be returned to the unfolded configuration to cover the interior space 182 and protect the instruments 120 and display 116 from unintentional contact. In other possible embodiments, the cover 602 is removable. In another possible embodiment, the cover 602 is retractable, such as using a spring to roll up the cover 602 into a temporary storage area.

In some embodiments, the lower housing 174 includes a customizable tray 604. The tray 604 forms the lower boundaries of the interior space 182, and includes customized features designed for a particular use of the patient care unit 102. For example, the tray 604 can include receptacles forming cradles for one or more instruments 120, as discussed herein. In some embodiments, the tray 604 includes one or more electrical connectors configured to connect with electronic components of the patient care unit, to transfer electrical signals between the patient care device 103 and the instruments 120. In yet other embodiments, the tray 604 includes apertures that provide access to the electronic components—for example, to allow an electrical connector from an instrument 120 to be plugged into a corresponding receptacle of the patient care device 103 electronics.

The customizable tray 604 configuration is particularly advantageous when the patient care unit 102 is manufactured for multiple different uses. For example, one patient care unit 102 may be designed and configured for use by patients having asthma (and therefore include a particular collection of instruments that are useful in managing, monitoring, or treating asthma), while another patient care unit 102 may be designed and configured for use by patients having heart problems (therefore including a different set of instruments 120), and yet another patient care unit 102 may be designed and configured for use as a medicine management system (therefore including a pill management system and perhaps one or more other instruments 120). Because each configuration of the patient care unit 102 is different, a variety of trays can be designed to contain the appropriate set of instruments.

A further advantage of the customized tray 604 configuration, however, is that the rest of the patient care unit 102 can be identical regardless of the intended use of the patient care unit 102. This results in manufacturing efficiency and reduced cost, because a large number of the same patient care units 102 can initially be manufactured (without the specific instruments or tray 604), and the customized trays 604 can then be inserted along with the appropriate instruments 120, without requiring the completely separate manufacturing of many different models of patient care units 102.

Once the tray 604 is inserted into the lower housing 174, it can be latched or otherwise fastened in place (e.g., with adhesive, screws, or the like). In some embodiments the tray 604 is removable, such as to permit replacement of the tray 604 with a different tray 604. Such as to upgrade or replace instruments 120, or to permit replacement of one type of tray (e.g., an asthma tray) with another type of tray (e.g., a pill management tray).

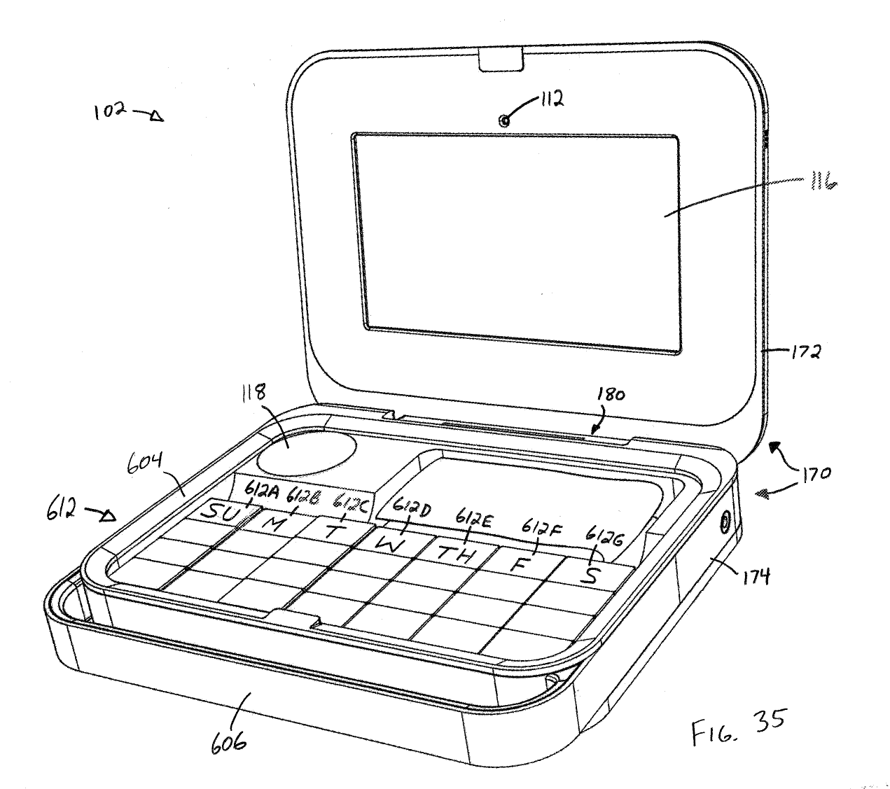

In some embodiments the housing 170 includes a carrying handle 606 for transporting the patient care unit 102, when the patient care unit 102 is in the closed position. In this example, the handle 606 is formed in the lower housing 174, and can be extended by pulling the handle 606 forward to extend it from the housing 174 (as shown in FIG. 35). It can then be grasped with a hand to conveniently carry and transport the patient care unit 102 from one place to another. The handle 606 can then be returned to the storage position, by moving the handle 606 rearward until the handle 606 is in the position shown in FIG. 34.

FIGS. 35-38 illustrate another example of the patient care unit 102, in which the patient care unit 102 is, or includes, a medication management system. This example of the patient care unit 102 can share many features with other examples of the patient care unit 102, described herein, and many such features are not repeated here.

FIG. 35 is a perspective view of the example patient care unit 102. In this example, the patient care unit 102 includes a housing 170 with an upper housing 172 and a lower housing 174 pivotally connected by a hinge 180. In some embodiments, the patient care unit 102 includes a tray 604 (described in more detail herein with reference to FIG. 34), which is designed and configured to contain components of the medication management system. The housing 170 includes the handle 606, as also discussed with reference to FIG. 34, and the handle 606 is illustrated in FIG. 35 in the extended position.

The medication management system includes one or more pill organizers 612. In some embodiments, the medication management system includes seven pill organizers—one for each day of the week, including pill organizers 612A (Sunday), 612B (Monday), 612C (Tuesday), 612D (Wednesday), 612E (Thursday), 612F (Friday), and 612G (Saturday).

In some embodiments, the pill organizers 612 each include a single receptacle, while in other embodiments the pill organizers 612 include multiple receptacles (such as in a range from 1 to 24). The example shown in FIG. 35 includes pill organizers 612 having four receptacles. The four receptacles can be assigned to particular times or portions of the day, such as breakfast, lunch, dinner, and bedtime. Other quantities of receptacles can be used to divide up the day into other intervals, such as one receptacle (daily), four receptacles (four times per day), or even 24 receptacles (hourly), for example.

In some embodiments, the pill organizers 612 are each removable from the patient care unit 102. For example, if a patient is planning to be traveling for the weekend, the patient may choose to remove pill organizers 612F and 612G from the patient care unit 102 so that they can be transported over the weekend without having to transport the entire patient care unit 102. When the patient returns, the pill organizers can then be returned to the patient care unit 102.

In some embodiments, each pill organizers 612A-G are keyed to match corresponding keyed receptacles of the tray 604. The keyed pill organizers 612A-G each have a different and unique shape, which only matches the corresponding shape of one keyed receptacle of tray 604, so that the pill organizers 612A-G can only be placed into the tray 604 in the proper positions. For example, if the Saturday pill organizer 612A and Sunday pill organizer 612G are both removed from the positions shown in FIG. 35, they can only be returned to those same positions. If the patient attempts to return the pill organizer 612G to the position for pill organizer 612A, the pill organizer has a shape that does not match the corresponding shape of the receptacle of tray 604 at that position, and therefore the pill organizer 612G will not fit into that position. The keyed features of the pill organizers 612 and corresponding receptacles of tray 604 can include a variety of different protruding regions, pins, recessed regions, or other shapes that prevent pill organizers 612A-G from being inserted into all but the appropriate position in the tray 604.

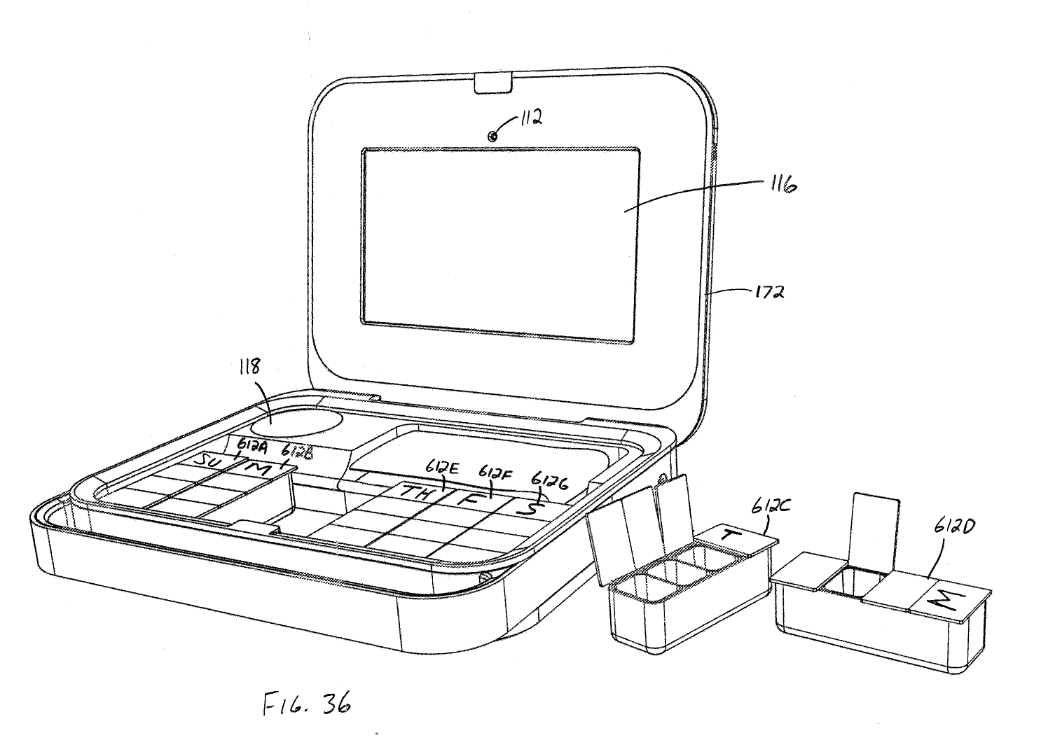

FIG. 36 is another perspective view of the patient care unit 102 shown in FIG. 35. In this example, the pill organizers 612C and 612D have been removed from the corresponding positions within tray 604.

In addition to storing and organizing pills within the pill organizers 612, the patient care unit 102 also intelligently assists the patient in managing the pills. For example, in some embodiments the patient care unit 102 interacts with the user through the user interface on the display 116 and through voice instructions through the speaker 118 to guide the user through: properly placing pills in the pill organizers 612, and removing and taking pills from the pill organizers 612 at the proper times and providing appropriate reminders when pills are to be taken, or alerts if pills are not taken. Some embodiments also provide access to information from a pharmaceutical database, to educate or remind the patient about certain pills, or answer questions that the patient may have about the pills.

In some embodiments, the patient care unit 102 generates a user interface that instructs the patient step-by-step to properly load pills into the pill organizers. The instructions can be customized based on known prescriptions for the patient, for example, that identify the particular pills, dosage, and timing, and such information is used to generate customized loading instructions.

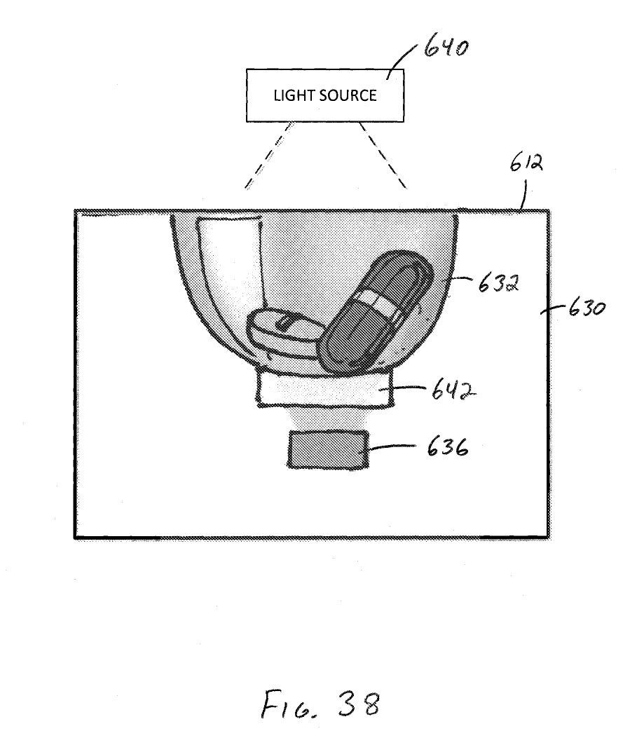

One example loading process is as follows. The patient is prompted to locate a particular pill bottle associated with a first prescription. The patient is then prompted to hold up the pill bottle in front of the video camera 112, at which time the bottle is scanned. A barcode on the bottle is scanned, or alternatively, the text on the bottle is recognized and read. The dosage instructions are then determined, and the patient is instructed to place the pills into the appropriate receptacles of the pill organizers 612. For example, if a pill is to be taken each morning, the patient can be prompted to open the morning receptacle for each pill organizer 612, and to place one pill into each receptacle. To further assist the patient, the user interface displays a graphical representation of the pill organizers 612 and highlights the appropriate receptacles. In another possible embodiment, one or more light sources (such as located below the pill organizers) are used to illuminate the actual one or more receptacles in which the pills should be placed. The patient care unit 102 can then prompt the user to indicate when the pills have been loaded, or can automatically detect the placement of the pills in the receptacles, as discussed in more detail with reference to FIG. 38. The process can be repeated for all pills until the pill organizers 612 have been appropriately loaded with pills according to the prescriptions.

The patient care unit 102 can also manage non-prescription drugs (including vitamins and other supplements), in which case the instructions are determined based on inputs from the user or recommendations of a caregiver, or based on dosage recommendations from the drug manufacturer.

In addition to (or instead of) intelligent and automated assistance that may be provided by the patient care unit 102, the patient care unit 102 can also provide access to a healthcare professional that can assist the patient with managing their pills. For example, a live audio and/or video session can be held through the patient care unit 102 during which the healthcare professional guides the patient through the loading of the pill organizers 612 and/or to assist the patient in taking the appropriate pills at the appropriate times. In some embodiments, the healthcare professional can be called by the patient by selecting a Call option in the user interface to obtain help or answer any questions that the patient may have while interacting with the medication management system (or any other portion) of the patient care unit 102.

In some embodiments, the video camera 112 can be used to provide the healthcare professional with a view of the pill organizers 612. In some embodiments, the video camera 112 is pivotally connected to the upper housing 172 so that the video camera can be manually pivoted to point downward toward the pill organizers, or alternatively the pivot can be motorized to permit the patient care unit 102 to pivot the video camera. In another possible embodiment, a second external video camera is positioned to have a view of the pill organizers 612, such as through a video headset or with a tripod mounted video camera.

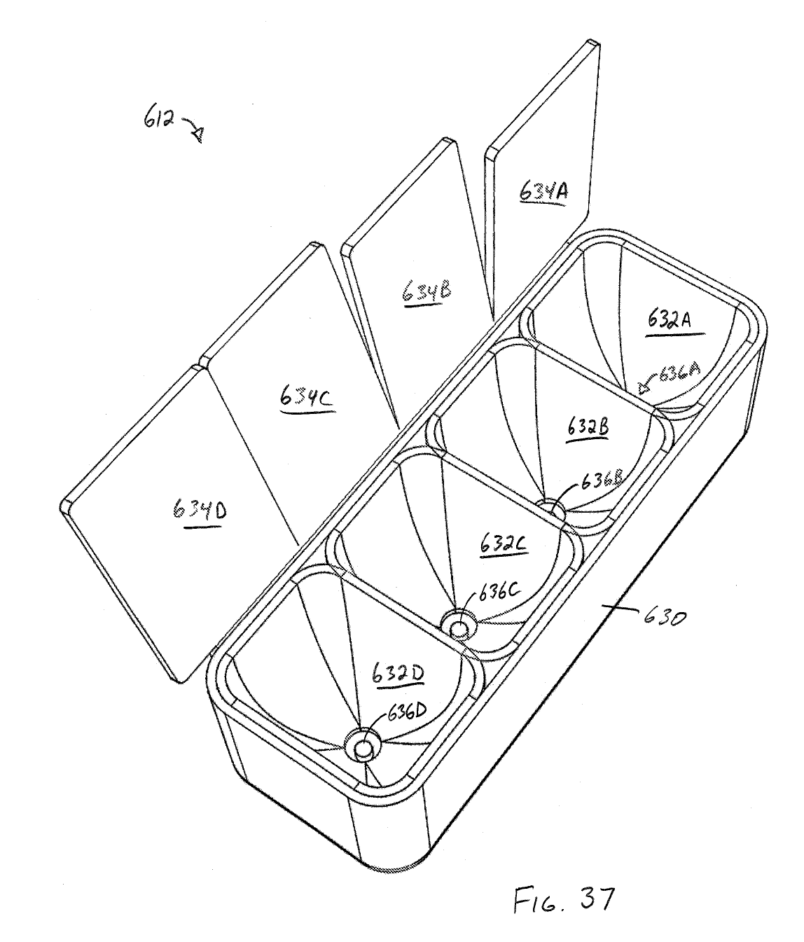

FIG. 37 illustrates an example of a pill organizer 612. The example pill organizer 612 includes a body 630, receptacles 632, and lids 634. Some embodiments further include pill detectors 636.

The body 630 of the pill organizer 612 is typically formed of a washable material such as plastic. Other materials are used in other embodiments. In some embodiments the pill organizer 612 is removable from the patient care unit 102. Further, in some embodiments the body 630 has a keyed feature, which is a part with a unique shape that will permit the pill organizer 612 to be inserted into only one location of the tray 604, shown in FIG. 36, which has a shape configured to receive the keyed feature.

Receptacles 632 are formed in the body 630. This example includes four receptacles 632A-D, though other embodiments have other quantities of receptacles. The receptacles are sized to store pills therein. In some embodiments, the interior surfaces of the receptacles are curved to slope downward toward a pill detector 636. This causes pills contained in the receptacles 632 to be pulled by gravity toward the pill detectors 636.

The pill organizer 612 also includes one or more lids 634. When the lids 634 are closed, the lids 634 enclose the receptacles 632, such as to prevent the pills from falling out of the receptacles 632. The lids 634 can be opened to remove the pills from the receptacles.

In some embodiments, pill organizers 612 include child safety locks. The child safety locks conform to a standard child resistant packaging standard, such as ISO 8317, ISO 13127, or ASTM D3475.

In some embodiments, the entire patient care unit includes a child safety lock, such as to make it difficult for a child to open the upper housing 172 from the lower housing 174 and thereby gain access to the pill organizers 612. The child safety lock can include, for example, latches positioned on opposite sides of the patient care unit 102 that must both be simultaneously released in order to open the patient care unit 102. In another embodiment, the lock is a key, a combination lock, or other locking mechanism that is difficult for a child to open.

In another possible embodiment, the cover 602 (shown in FIG. 34) includes a child safety lock, where the cover 602 encloses the interior space 182 and the pill organizers 612 therein. Some embodiments utilize child resistant locks or latches. In yet another possible embodiment, the cover 602 is locked by the patient care unit 102 until a password or other input is provided through the touch-sensitive display 116, at which time the patient care unit 102 unlocks the cover 602 to permit access to the pill organizers 612.

Some embodiments further include pill detectors 636, which are illustrated and described in more detail with reference to FIG. 38.

FIG. 38 is a schematic cross-sectional view of a receptacle 632 of a pill organizer 612, and including a pill detector 636.

In this example, the receptacle 632 includes pills. The interior surface of the receptacle is curved to direct the pills toward a lower central region of the receptacle 632. The pill detector 636 is arranged below this lower central region of the receptacle 632 and detects the presence or absence of pills in the receptacle 632. In some embodiments the pill detector 636 is contained within the body 630 of the pill organizer 612, as shown in FIG. 38, while in other embodiments the pill detector 636 is located within the patient care unit 102, such as directly below the storage positions of the pill organizers 612.

An example of a pill detector 636 is a light sensor. The light sensor operates to detect light from a light source 640, such as positioned above the receptacle 632. An example of a light source is a light emitting diode or a light bulb. The light source can be contained in the patient care unit 102, on either the upper 172 or lower housing 174. In another possible embodiment, the light source is part of the pill organizer 612. In another possible embodiment, the light source is ambient light. The light source can also be part of or located adjacent to the pill detector 636.

As one example of the operation of the pill detector 636, the light source illuminates the receptacle 632. When the pills are absent from the receptacle 632, the light passes through the receptacle and is detected by the pill detector 636. When the pills are present in the receptacle 632, the pills block at least some of the light, thereby decreasing the amount of light detected by the pill detector. This difference in light permits that pill detector (or the patient care unit 102) to determine whether each of the receptacles 632 of each of the pill organizers 612 contain pills.

In some embodiments, the body 630 includes a transparent window 642 positioned at the lower central region of the receptacle 632, and the pill detector 636 is positioned below this window 642.

The pill detector 636 transfers a signal or data to the electronics of the patient care unit 102 via wires or wirelessly. The patient care unit 102 then uses this information to help the patient manage his or her medications.

In some embodiments, a separate light source 640 is provided for each receptacle 632, which operates to illuminate a single receptacle 632 to identify that receptacle 632 for the patient, such as to identify the receptacle 632 as containing pills that should be taken by the patient, or to identify a receptacle 632 in which pills should be placed.

In some embodiments, the medication management system operates to assist the patient in managing the taking of medication when the patient travels to a location with a different time zone. When the patient travels to such a location, it can be difficult for the patient to know when to take his medication—for example, does he need to stay on his regular schedule, or can he simply wait several hours and resume according to his normal schedule? The medication management system can be used to determine the appropriate course of action. For example, the medication management system accesses pharmaceutical databases, and retrieves information associated with the prescription medications that the patient is currently taking. The information in the database indicates whether timing is crucial for such prescriptions, and/or whether it is OK to take a next dose of the medication early or late. The medication management system then presents a recommendation to the patient, and assists the patient in following the recommendation, by providing suitable reminders at the appropriate time.

Similarly, the medication management system can also assist the patient in determining the best course of action when the patient does not take one or more prescription medications on time. For example, the medication management system may advise the patient to take the medication now, or may recommend that the patient skip that dose and resume taking the medication at the next scheduled time.

Reminders and alerts can be provided by the patient care unit 102, or by a remote computing device, or by a remote healthcare professional who is alerted by the telemedicine care system 100. The reminders and alerts can be provided by e-mail, text message, telephone call, calls through the patient care unit, audible or visible alarms through the patient care unit 102, or by alerting another person (e.g., a family member or other care provider) who can then assist the patient.

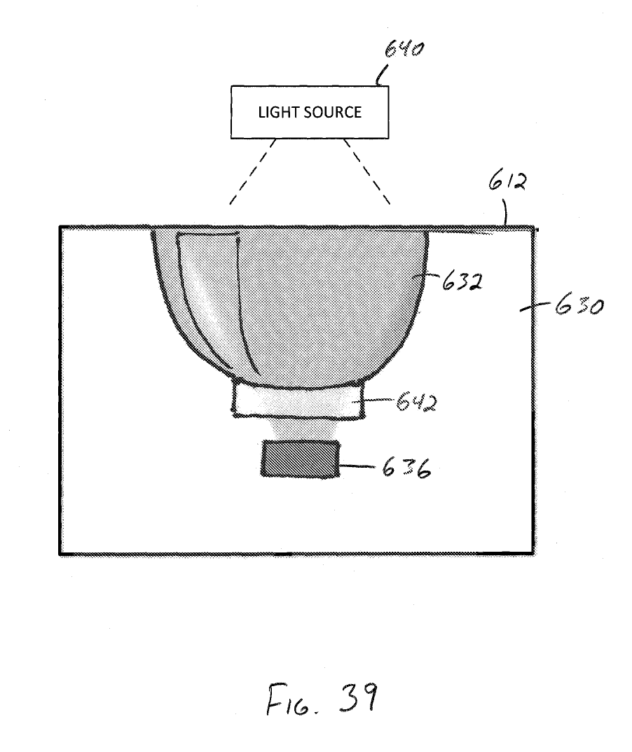

FIG. 39 is a schematic cross-sectional view of the receptacle 632 of the pill organizer 612 shown in FIG. 38, without pills in the receptacle.

FIG. 40 is a schematic diagram illustrating exemplary communication devices of the patient care unit 102. In some embodiments, the patient care unit 102 includes at least one audio communication system 662 and at least one separate data and video communication system 664. Some examples of suitable communication devices are illustrated and described herein with reference to FIG. 7.

When a patient and a remote healthcare provider are communicating with each other, the most important aspect of that communication is the voice communication. If either of the voice channels is delayed or of inadequate sound quality, the communication between the patient and the healthcare professional is severely impacted.

As a result, some embodiments include a separate audio communication system 662 is provided, which handles the voice communication separate from the video and data communication to provide high quality audio communications and reduced latency than may otherwise occur if audio, video, and data were all transmitted together.

Further, some embodiments include multiple communication devices. For example, the audio communication system 662 includes a wired telephone communication device 670 and a cellular communication device 672. The wired telephone communication device 670 is an integrated telephone, which can be connected to an external telephone cable connected to a switched telephone network. The cellular communication device is an integrated cellular telephone, which can communicate wirelessly through a cellular telephone network.

In some embodiments, the audio communication system 662 selects from the available audio communication devices according to priority. For example, the wired telephone communication device 670 is assigned a higher priority than the cellular communication device 672 in some embodiments. Accordingly, if the wired telephone communication device 670 is available for use (such as by being connected to the switched telephone network), the audio communication system 662 utilizes the wired telephone communication device 670 for voice communications. If it is not available, then the audio communication system 662 utilizes the cellular communication device 672 for voice communications. If the cellular communication device 672 is also not available, then the audio communication device may notify the user, or alternatively may choose to utilize the data and video communication device to transfer voice communications across another network, if such network is available for communication. Further, in some embodiments the data and/or video communication may be temporarily suspended by the patient care unit in this case to avoid interfering with the voice communication.

In some embodiments, the data and video communication system 664 also includes multiple communication devices. In this example, the data and video communication system 664 includes a wired communication device 674, a Wi-Fi communication device 676, and a cellular data communication device 678. An example of a wired communication device is an Ethernet communication device, which can be connected to a local area network through an Ethernet cable, for example. An example of a Wi-Fi communication 676 is a Wi-Fi radio transceiver. An example of a cellular data communication device 678 is a 4G modem, or other similar cellular data communication device.

In some embodiments, the data and video communication system 664 selects from the available data and video communication devices according to priority. For example, the order of priority (from highest to lowest) may be: the wired communication device 674, the Wi-Fi communication device 676, and the cellular data communication device 678. If none of the video communication devices are available to transmit data and video, the data and video communication system 664 typically will not attempt to communicate the data and video using the audio communication device, because such communication may interfere with, or decrease the quality of, the voice communication.

In another possible embodiment, the wireless communication device 676 can access the cellular network by communicating with an external cell phone configured to provide internet sharing services (sometimes referred to as a Personal Hotspot).

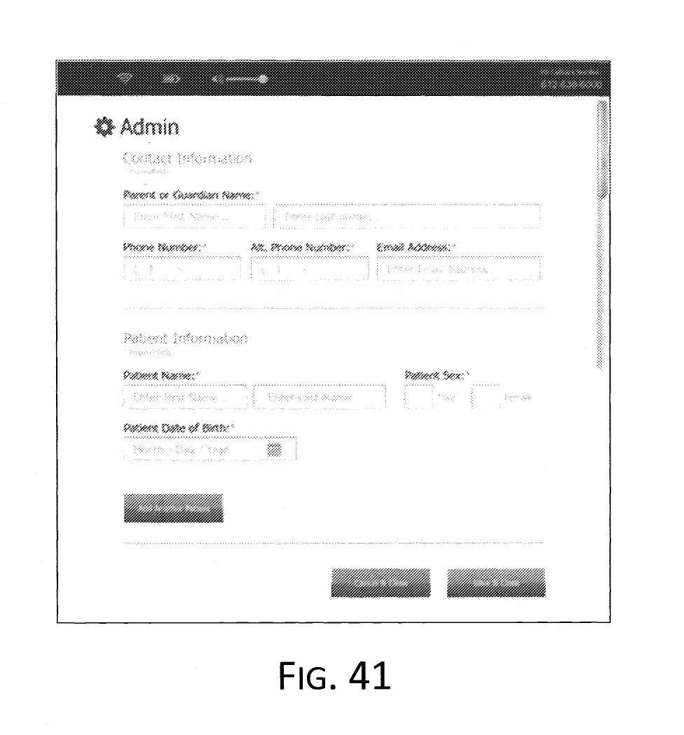

FIGS. 41-57 illustrate additional exemplary user interfaces example patient care units 102, and associated functions that can be performed by some embodiments of the telemedicine care system 100. The illustrated examples show user interfaces intended for use by a patient having asthma.

In some embodiments, the user interface is generated by a software application running on the patient care unit 102. In another possible embodiment, the user interface is generated by a remote server which sends web page data to the patient care unit 102. The patient care unit 102 utilizes a browser software application to display the user interface based on the web page data. Other embodiments utilize other configurations.

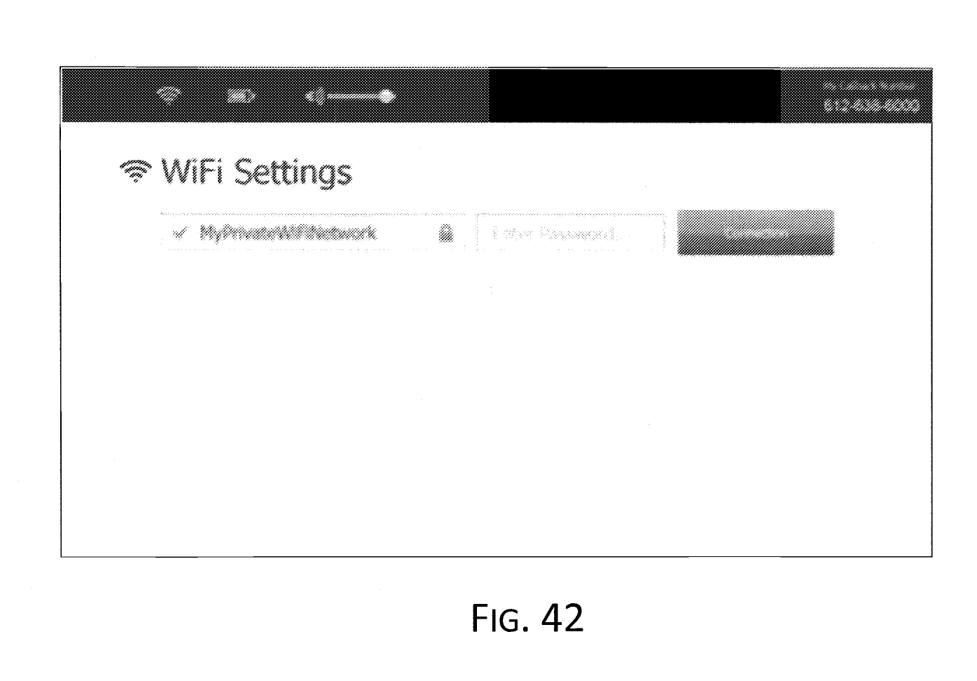

FIGS. 41-42 illustrate an example setup process.

FIG. 41 is a screen shot of an example admin page of the user interface. The admin page prompts the user to provide patient information. In this example, user is prompted to enter contact information, and patient information. The patient information can be used, for example, to retrieve additional information about the patient, or simply to identify the patient in communications with the remote healthcare professional.

FIG. 42 is a screen short of an example Wi-Fi settings page of the user interface. The Wi-Fi settings page is used to obtain information about the local Wi-Fi settings, if available. For example, the user selects or enters an SSID for the Wi-Fi access point, and enters a password.

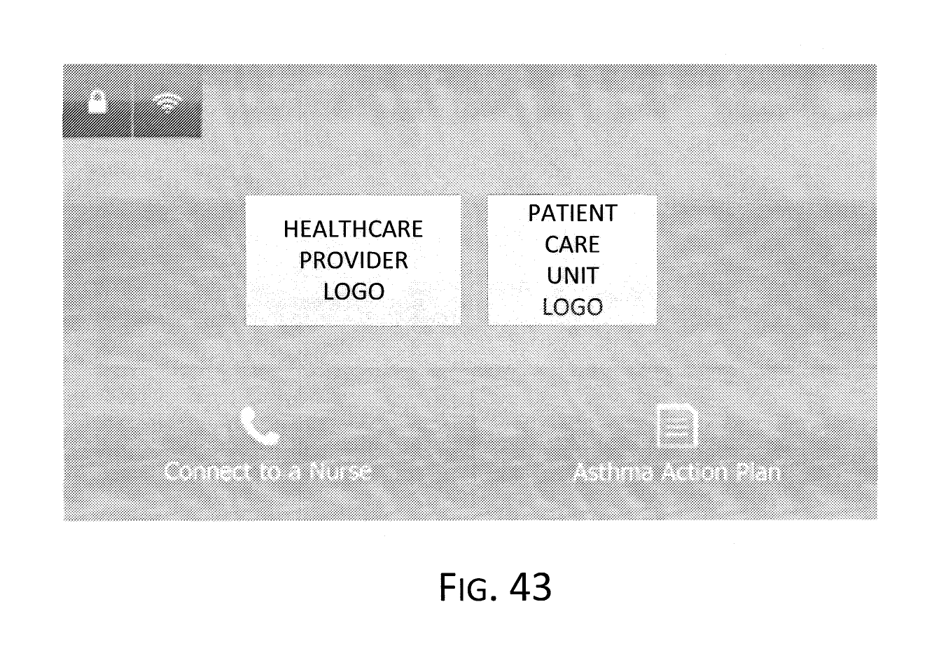

FIGS. 43-45 illustrate an example process of connecting with a remote healthcare professional.

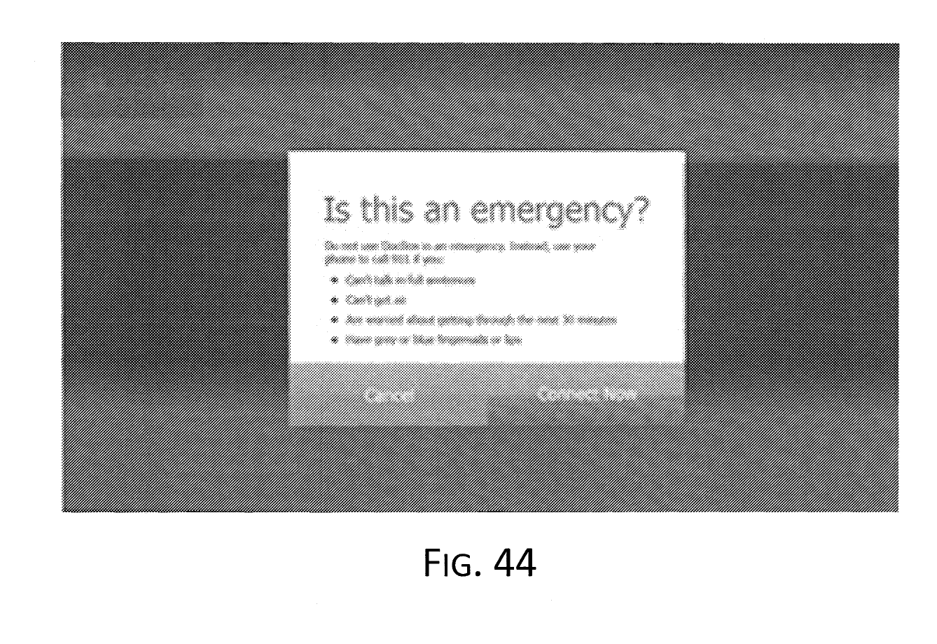

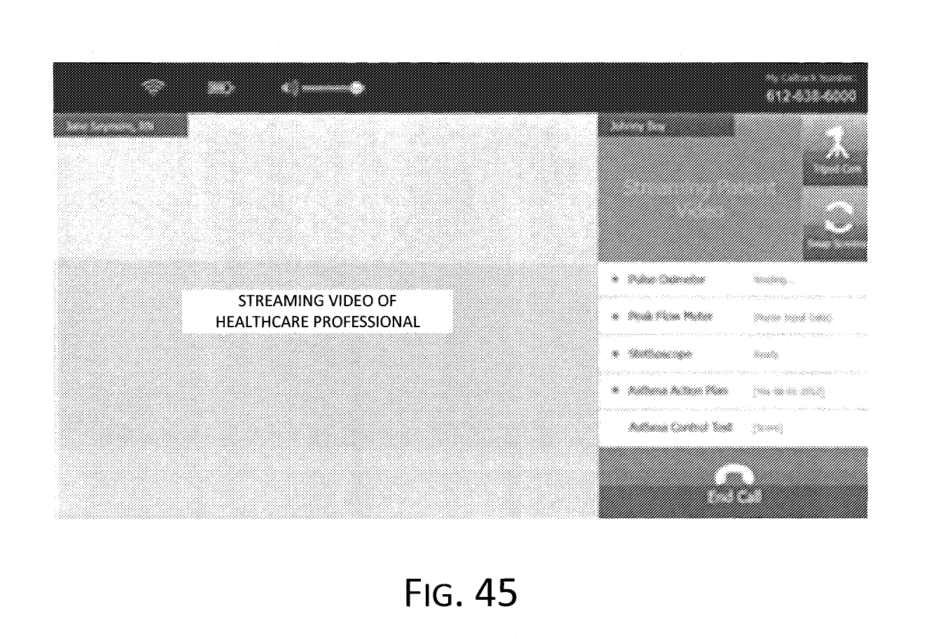

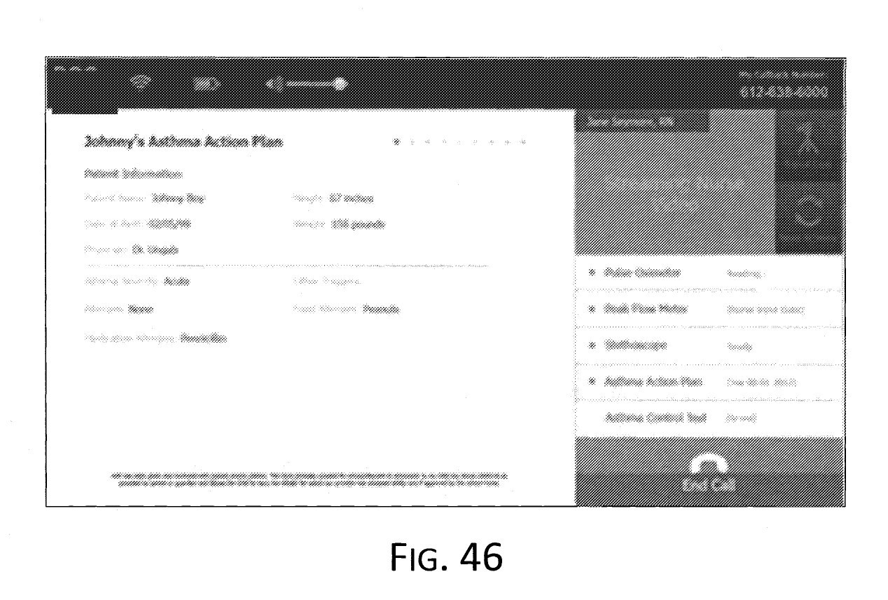

FIG. 43 is a screen shot of an example home page of the user interface. The home page is the main page that is displayed when the patient care unit 102 is started, after the initial setup process illustrated in FIGS. 41-42. The home page prompts the user to select whether to: connect to a nurse, or view an asthma action plan, for example. FIGS. 44-45 illustrate the user interface involved with connecting to a nurse, while FIGS. 46-52 illustrate example user interfaces for generating and viewing an asthma action plan.

FIG. 44 is a screen shot of an example validation screen of the user interface. In this example, the validation screen asks the user whether there is an emergency, and provides instructions for how to proceed (e.g., call 911) if there is an emergency. The validation screen then prompts the user to “cancel” or “call now.” A further advantage of the validation screen is to reduce the chance or accidental calls that may otherwise occur without the validation screen. If the user unintentionally selects the “connect to nurse” option shown in FIG. 43, the user can now select “cancel” to avoid placing the call.

FIG. 45 is a screen shot of the live conference page of the user interface. The live conference page displays the live streaming video of the healthcare professional. In some embodiments, the live conference page also displays a “thumbnail” version of the patient video steam so that the patient can see what the healthcare professional is currently seeing. Selection buttons are provided to permit the user to select between multiple views or multiple different video streams in some embodiments. The user interface also includes an option to end the call.

Instrument data is also displayed in some embodiments. The instrument data window displays status information for the instruments, and/or data collected by the instruments. For example, the instrument data window shows that the pulse oximeter is currently taking a reading, the peak flow meter is awaiting input from the healthcare professional to enable its use, and that the stethoscope is ready for use. Once the pulse oximeter completes the reading, the result is displayed in some embodiments. Additionally, the user interface shows that an asthma action plan was last updated on a certain date, and that the last asthma control test that was completed resulted in a certain asthma control test score.

FIGS. 46-50 illustrate an example process for generating an asthma action plan. In this example, a healthcare professional works with the patient periodically (such as once per year) to generate an asthma action plan. The asthma action plan provides instructions to the patient on how to manage his or her asthma as the patient's condition changes. For example, if the patient is doing well, the asthma action plan may advise the patient to continue doing what he is doing. If the patient is doing poorly, the asthma action plan may recommend that the patient try something different to try to better manage the condition.

FIGS. 46-50 illustrate the user interface as displayed by the patient care unit 102. A corresponding user interface is also displayed to the healthcare professional at the healthcare professional computing device, who works with the patient to complete the asthma action plan.

FIG. 46 is a screen shot of an example patient information page of the user interface for the asthma action plan. The patient information page includes patient identification information, as well as other medically relevant information about the patient, such as the severity of the patient's asthma, known allergies, allergies to medications, a list of known triggers to the asthma, and known food allergies. The healthcare professional reviews this information with the patient and enters or updates information as necessary.

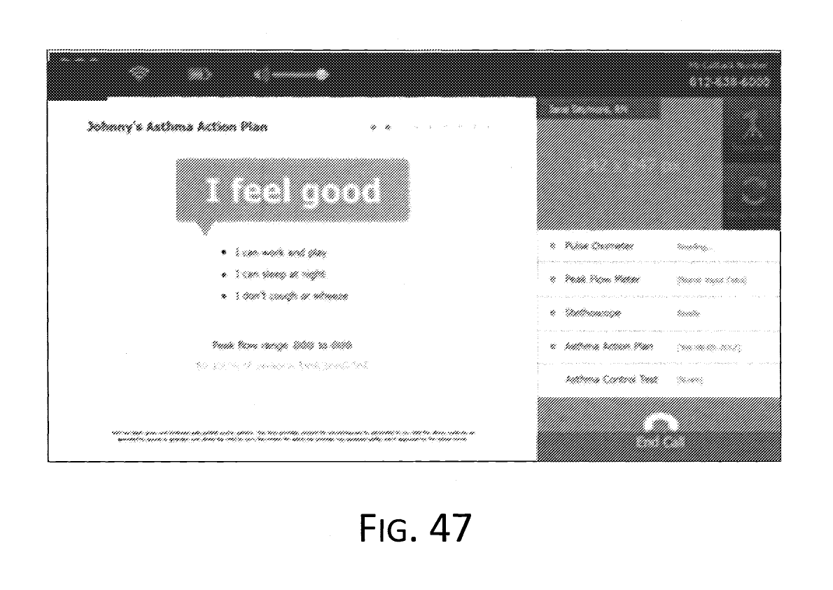

FIG. 47 is a screen shot of a first condition page of the user interface. In this example, the first condition is “I feel good.” The first condition page provides an explanation of how the patient should feel when he is in the first condition (e.g., “I can work and play,” “I can sleep at night,” and “I don't cough or wheeze”), and the healthcare professional reviews this with the patient to ensure that the patient understands this condition. The healthcare professional may also set a peak flow range associated with the first condition.

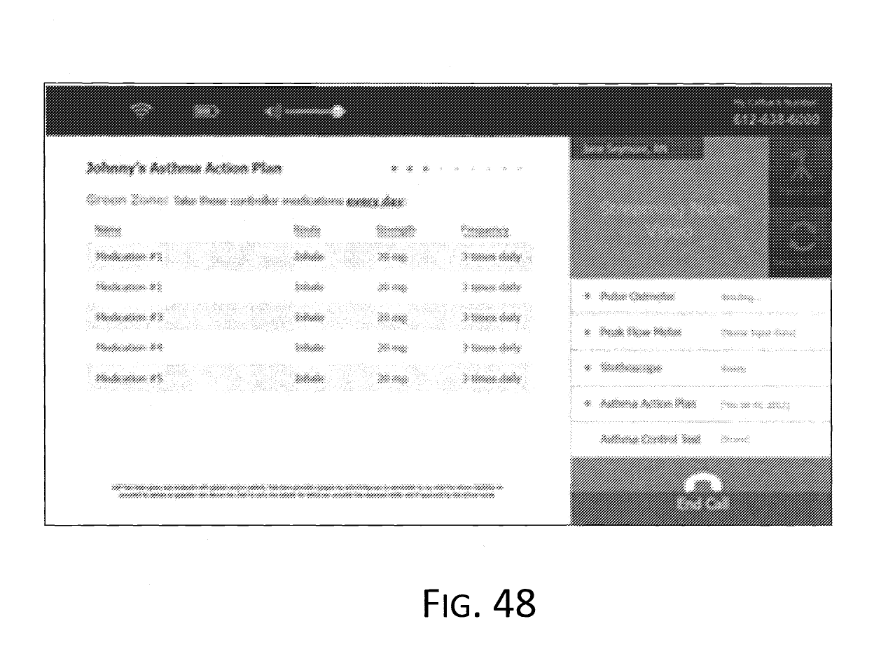

FIG. 48 is a screen shot of a medication page associated with the first condition. The healthcare professional identifies the medications that the patient should take when the patient has the first condition, which are then displayed to the patient through this medication page associated with the first condition. In this example, the medication page displays a list of the medications, along with a route (e.g., inhale), strength (e.g., 20 mg), and frequency (e.g., 3 times per day).

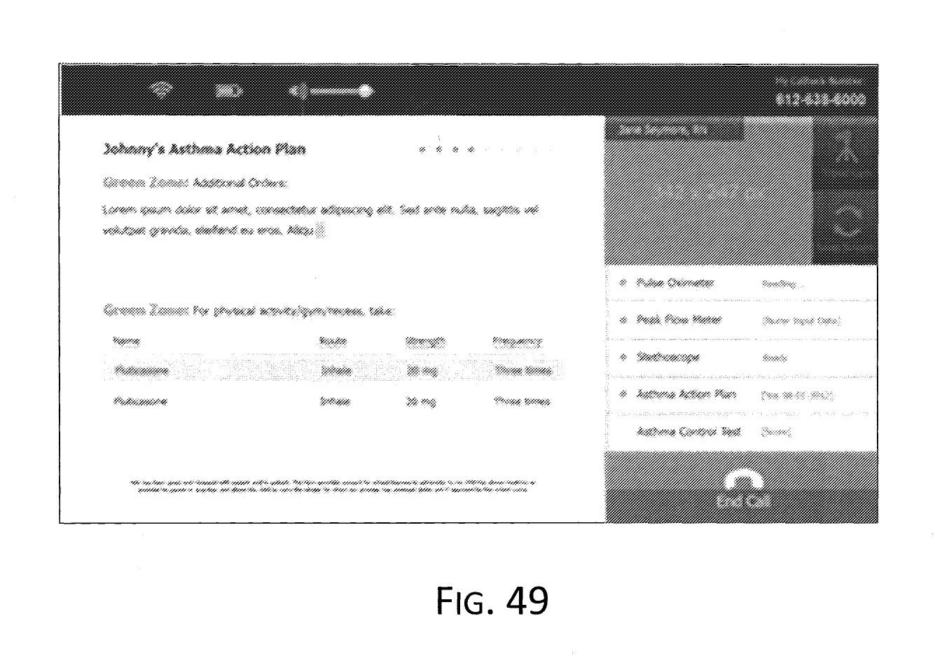

FIG. 49 is a screen shot of an additional actions page associated with the first condition. The healthcare professional works with the patient to identify additional orders, if any, that the patient should follow when he has the first condition, and any additional medications that should be used when certain events occur, such as prior to, during, or after physical activity.

The same process illustrated in FIGS. 47-49 is then repeated for other conditions, such as a second condition (“I do not feel good”), and a third condition (“I feel awful.”) The healthcare professional talks with the patient through this process to ensure that the patient understands what he is to do when various conditions occur.

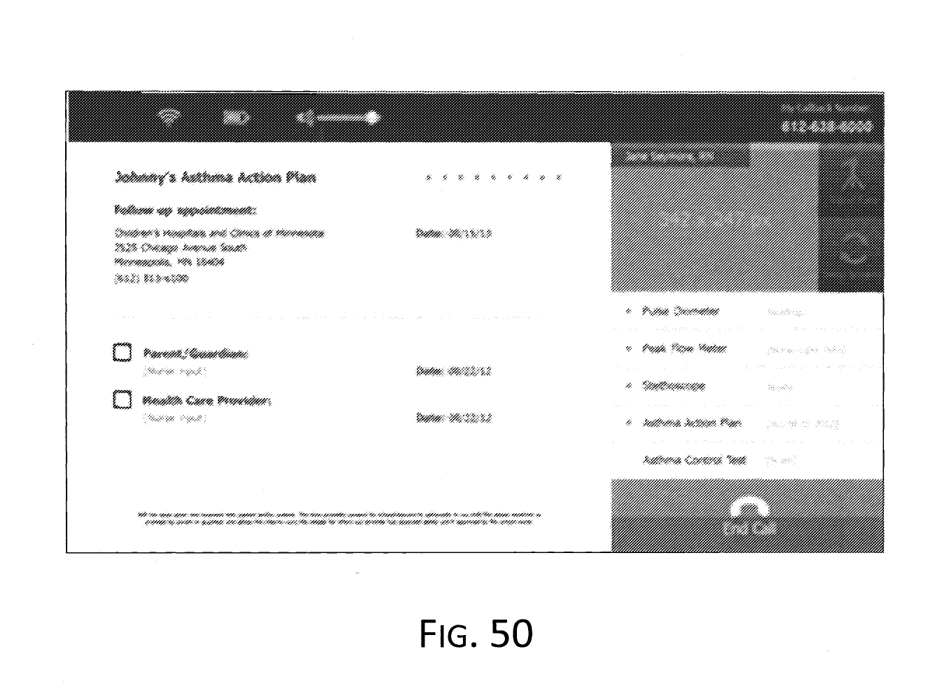

FIG. 50 is a screen shot of a follow up page of the user interface. The follow up page identifies a date and time for the next appointment, or may provide contact information that the patient should use to schedule a follow up appointment at a later date. An input is then requested from both the healthcare professional and the patient or guardian to indicate that both were present and participated in the generation of the asthma action plan on the current date. Upon completion of the asthma action plan, it is saved on the patient care unit 102, on a remote server, and/or in the patient's medical record for subsequent use.

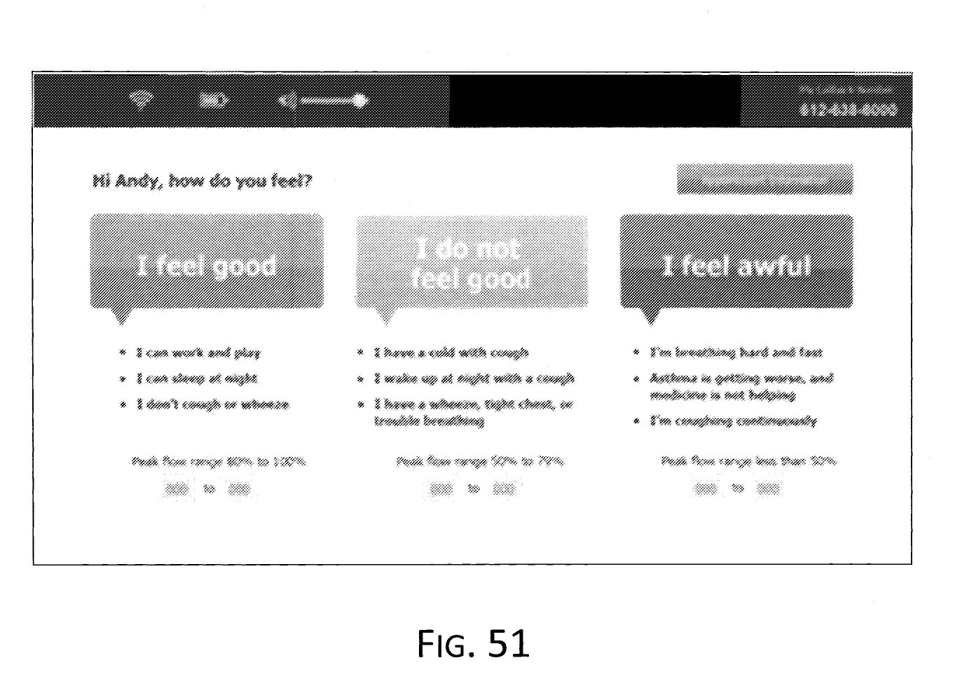

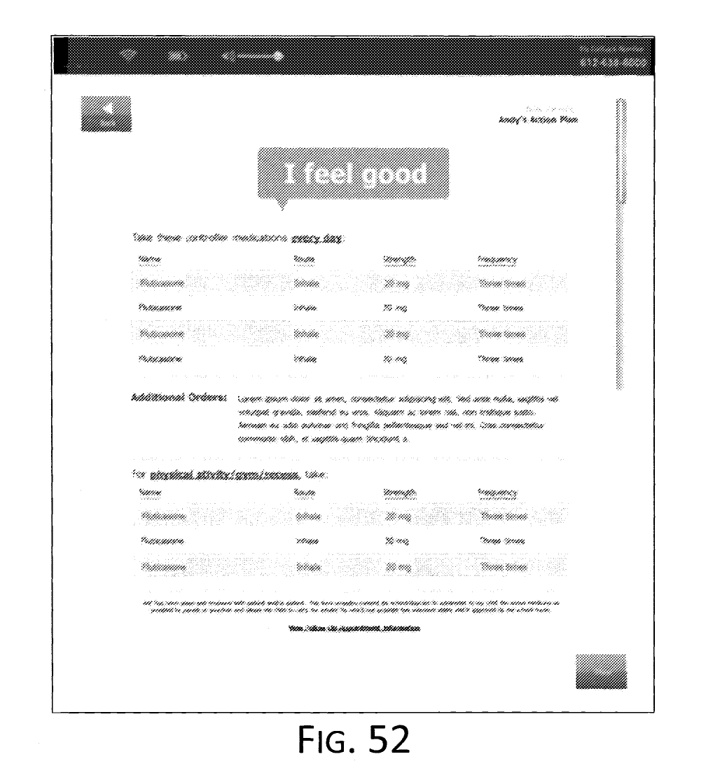

FIGS. 51-52 illustrate a process of presenting an asthma action plan to a patient through a user interface of the patient care unit 102.

Referring briefly to FIG. 43, when a patient wants to review the asthma action plan, the patient selects the asthma action plan option from the user interface. The user interface then displays a condition prompting page, as shown in FIG. 51.

FIG. 51 is a screen shot of an example condition prompting page of the user interface, as displayed on the patient care unit 102. The condition prompting page prompts the user to identify how he feels from several different conditions. Upon selection of a condition, a treatment page is then displayed, such as shown in FIG. 52.

In another possible embodiment, the patient is asked to use a peak flow meter to permit the patient care unit 102 to get a peak flow reading. The peak flow reading is then used to automatically identify, or suggest, a condition to the patient based on predetermined peak flow ranges associated with each condition.

An appointment information button is also provided. Upon selection, the user interface displays the follow up information, such as was shown in FIG. 50.

FIG. 52 is a screen shot of an example treatment page of the user interface. The treatment page displays the medications and additional actions that were identified by the healthcare professional for the selected condition, when generating the asthma action plan. The treatment page reminds the patient of the appropriate treatment for managing the asthma when he is in the selected condition.

Similar pages are displayed if the other conditions are selected, along with the corresponding medications and additional actions associated with that condition.

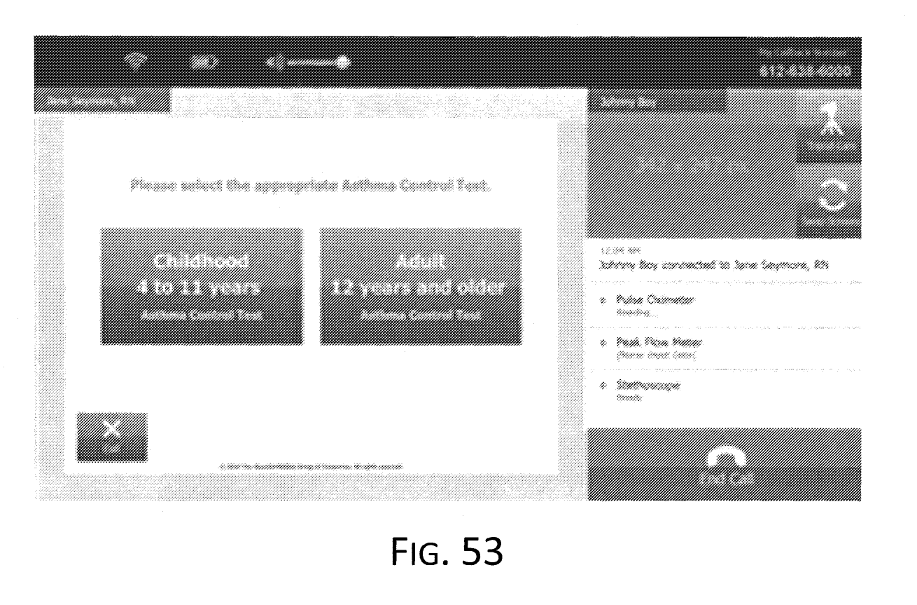

FIGS. 53-57 illustrate an example process of administering an asthma control test to a patient using the patient care unit 102.

FIG. 53 is a screen shot of an example control test selection page of the user interface. The control test selection page prompts the user to select an appropriate test. For example, the page prompts the user to determine whether the patient is a child or an adult. In other embodiments, the patient care unit 102 automatically determines the appropriate test using the patient information. In another possible embodiment, the patient care unit 102 makes only a single test available to the patient. The following Figures illustrate the test for a child.

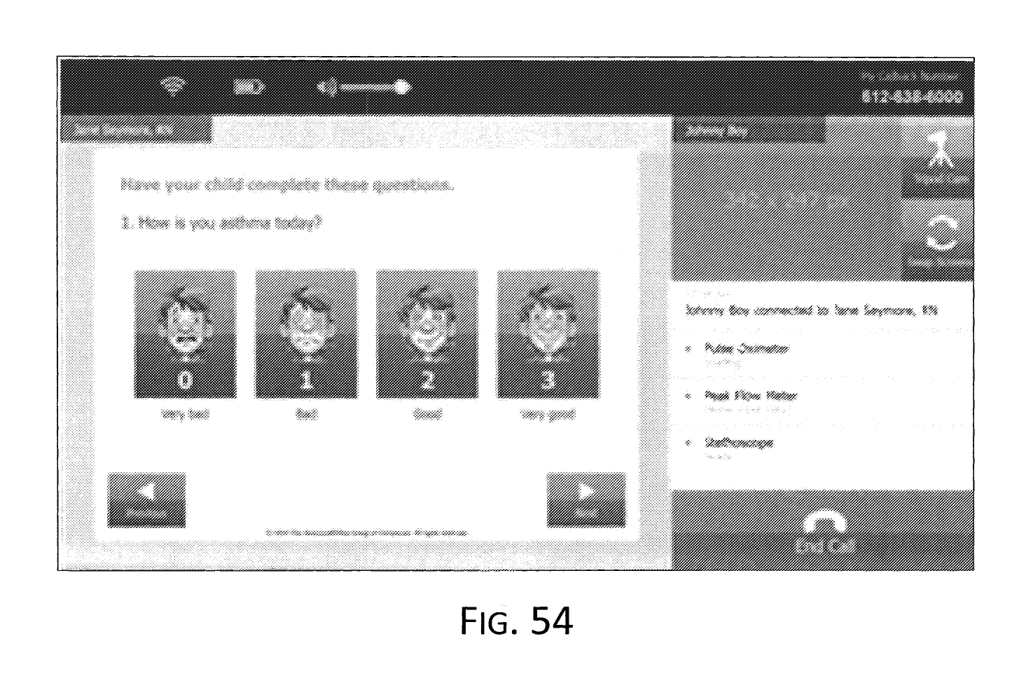

FIG. 54 is a screen shot of a test question page of the control test. In this example, a question is presented to the patient (which is also presented in an audible form in some embodiments), such as “1. How is your asthma today?” Several selectable controls are provided associated with various possible answers, including “0—very bad,” “1—bad,” “2—good,” and “3—very good.” Additionally, a graphical image is provided which is associated with each answer that further assists the child in answering the question. For example, the graphical image associated with “3—very good” depicts a boy that is smiling, while the graphical image associated with “0—very bad” depicts a boy with a grimace on his face who looks to be in pain. The user is prompted to select one of the answers by touching the answer through the touch-sensitive display. Additional questions may then be presented.

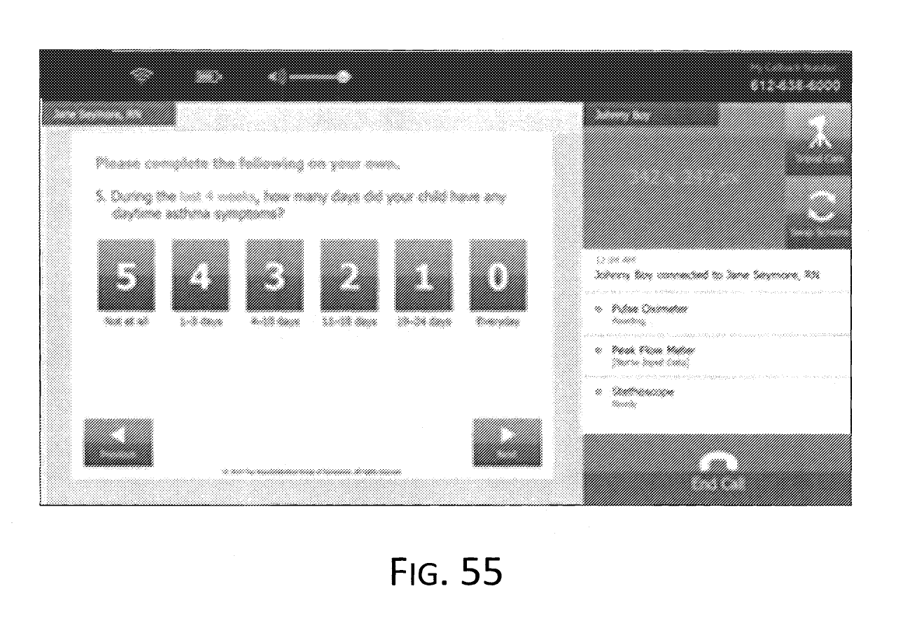

FIG. 55 is a screen shot of another test question page of the control test. In this example, a question is presented in a different form and the parent is asked to answer the question. The question states: “5. During the last 4 weeks, how many days did your child have any daytime asthma symptoms?” Selectable controls are provided associated with various possible answers, including “5—Not at all,” “4—2-3 days,” “3—4-10 days,” “2—11-18 days,” “1—19-24 days,” and “0—Everyday.” The user selects the answer by touching the appropriate button on the touch sensitive display. Additional questions may then be presented.

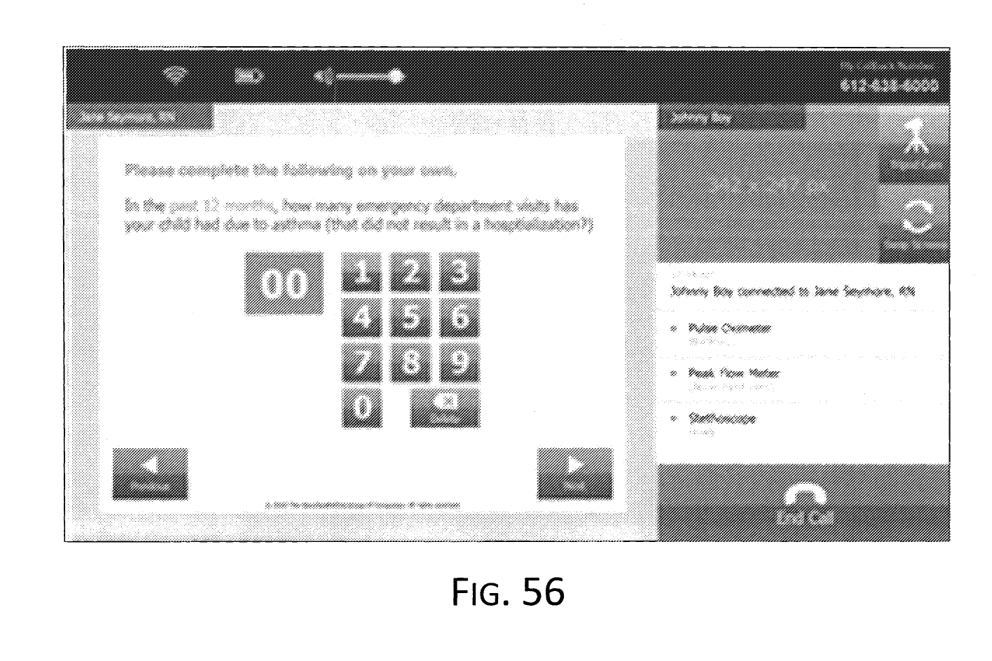

FIG. 56 is a screen shot of another test question page of the control test. In this example, a question is presented which requires a numerical input. A number pad is displayed on the touch sensitive display to permit entry of the numerical answer. The question states, “In the past 12 months, how many emergency department visits has your child had due to asthma (that did not result in a hospitalization)?” The number pad is used to enter the answer through the touch sensitive display.

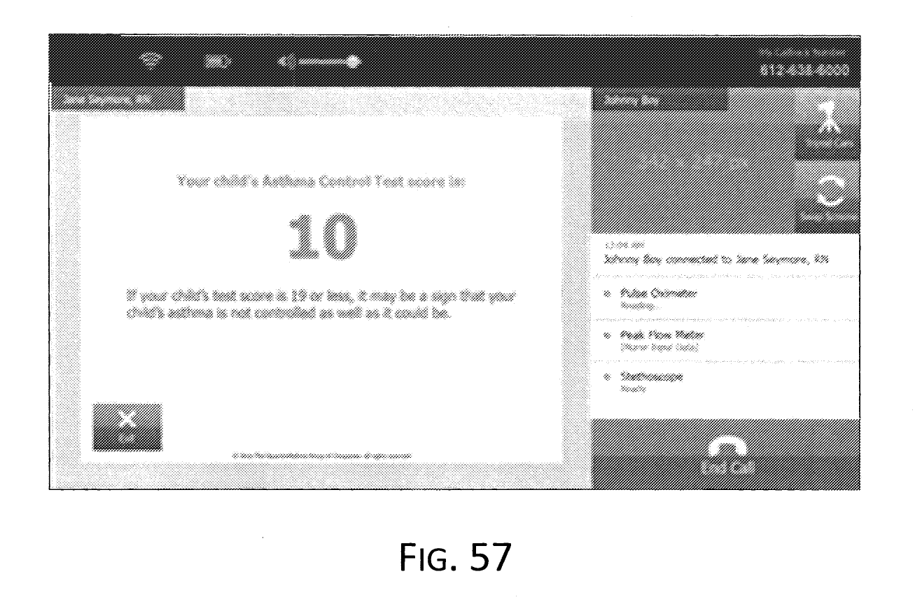

FIG. 57 is a screen shot illustrating the display of an asthma control test score. Upon completion of the asthma control test, the asthma control test score is computed according to a predetermined formula based on the answers to the test. In this example, the resulting asthma control test score was 10. A notice indicates that a score of 19 or less may be a sign that the patient's asthma is not controlled as well as it could be.

An advantage of administering the asthma control test through the patient care unit is that the administration and data collection can be automated. For example, the patient can be prompted to complete the test by a message on the home page, or by a message (e-mail, text, voicemail, call, etc.) or other alert. Upon completion of the test, the results are automatically sent a remote server where the data is collected and compiled from many different patients. The results can then be evaluated to generate statistics associated with the health care facility, such as to identify the relative effectiveness of the treatment of the condition by that facility.

Although multiple specific examples have been provided for interaction with patients with asthma, it is recognized that the patient care unit is in no way limited to patients with asthma. Various other conditions are described herein in other examples, and yet further embodiments can be used with patients having other medical conditions, and even patients having no known medical conditions.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A mobile patient care unit comprising:
a housing comprising:
a base portion;
a display portion; and
a hinge pivotally connecting the display portion to the base portion to permit the display portion to pivot with respect to the base portion between a closed position and an open position, wherein the housing encloses an interior space when the housing is in the closed position;
a touch sensitive display positioned in the display portion of the housing;
a video camera positioned in the display portion of the housing;
electronics at least partially contained by the housing, the electronics including:
at least one processing device;
a microphone;
a speaker;
a wireless communication device configured to wirelessly communicate across a digital data communication network;
a cellular communication device configured to wirelessly communicate using a cellular communication network; and
an instrument interface; and
at least one medical instrument configured to be stored within the interior space of the base portion of the housing, wherein the medical instrument is at least partially controlled by the electronics through the instrument interface.

2. The mobile patient care unit of claim 1, wherein the mobile patient care unit is configured to enable interaction between the patient and a remote caregiver, the mobile patient care unit being operable to:
interact with a patient at a location of the patient using the medical instrument and send data to be displayed to the remote caregiver;
display live video of a remote caregiver on the touch sensitive display;
capture live video of the patient using the video camera and send to the remote caregiver;
broadcast voice communications from the remote caregiver to the patient using the speaker; and
capture voice communications from the patient and send to the remote caregiver;

3. The mobile patient care unit of claim 1, wherein the electronics further comprise: a wired network communication device configured to communicate through a data communication network when coupled to the data communication network through a cable.

4. The mobile patient care unit of claim 3, wherein the cellular communication device operates to communicate the voice communications from the remote caregiver and from the remote patient across the cellular communication network, and wherein the live video is separately communicated through one of the wireless communication device and the wired network communication device, whereby the separate communication of the voice communications using the cellular communication device reduces possible latency of the voice communications.

5. The mobile patient care unit of claim 1, wherein the instrument interface comprises an instrument processing device separate from the processing device of the electronics and operable to interact with the medical instrument, wherein the instrument processing device is in data communication with the processing device of the electronics.

6. The mobile patient care unit of claim 1, wherein the wireless communication device comprises:
a wireless Wi-Fi radio configured to communicate according to a Wi-Fi data communication standard; and
a wireless Bluetooth radio configured to communicate according to a Bluetooth data communication standard.

7. The mobile patient care unit of claim 1, further comprising a second video camera in data communication with the electronics and unattached to the housing to permit movement of the second video camera separate from the housing.

8. The mobile patient care unit of claim 7, wherein the second video camera is part of a wireless headset.

9. The mobile patient care unit of claim 7, wherein the second video camera is mounted on a tripod.

10. The mobile patient care unit of claim 1, wherein the electronics and the medical instrument operate to generate data identifying at least one medically relevant characteristic of the patient.

11. The mobile patient care unit of claim 10, wherein the data identifying at least one medically relevant characteristic of the patient is communicated to by the mobile patient care unit separately from the live video to at least two separate servers.

12. The mobile patient care unit of claim 10, wherein the live video and the data are commonly communicated to one or more common servers.

13. The mobile patient care unit of claim 10, wherein the medically relevant characteristic of the patient is displayed on the display device.

14. The mobile patient care unit of claim 1, wherein the at least one medical instrument includes at least a wireless instrument and a wired instrument, both configured to be stored within the interior space, wherein the wireless instrument communicates with the electronics wirelessly through one of: the wireless communication device and the instrument interface, and wherein the wired instrument is electrically connected to the instrument interface by at least one cable.

15. The mobile patient care unit of claim 14, wherein at least the wired instrument and the electronics share a common power source, and wherein the wireless instrument, the wired instrument, and the electronics are all at least partially controlled by the processing device.

16. The mobile patient care unit of claim 1, wherein the at least one medical instrument comprises all of the following: a pulse oximeter, a wireless stethoscope, a digital spirometer, a peak flow meter, a weight scale, a blood pressure cuff, an otoscope, a wearable EKG device, a video headset, an inter-cardiac pressure detection device, an ear temperature thermometer, and a pulmonary edema detection device.

17. The mobile patient care unit of claim 1, wherein the at least one medical instrument comprises a wireless stethoscope having a surface, wherein the surface is configured to be removably fastened to a skin of the patient.

18. The mobile patient care unit of claim 1, further comprising a removable cover configured to cover the interior space to protect the touch sensitive display from contact with the one or more medical instruments.

19. The mobile patient care unit of claim 1, further comprising a medication management system including a plurality of pill storage receptacles, wherein a pill sensor is provided for each pill storage receptacle to determine whether pills are contained within the respective pill storage receptacles.

20. The mobile patient care unit of claim 1, wherein the patient care unit is adapted to:
display an asthma action plan to a patient as it is being generated by a remote caregiver, while simultaneously providing video and audio communication between the remote caregiver and the patient;
display the asthma action plan subsequent to its generation for use by the patient; and
administer an asthma control test to the patient to evaluate effectiveness of management of the patient's asthma.
